(19) United States
(12) Patent Application Publication
(10) Pub. No.: US 2024/0208964 A1
(43) Pub. Date: Jun. 27, 2024

(54) PYRIDOPYRIMIDINES AND METHODS OF THEIR USE

(71) Applicant: PYRIDOPYRIMIDINES AND METHODS OF THEIR USE, Seattle, MA (US)

(72) Inventors: Kerem OZBOYA, Cambridge, MA (US); Iwona WRONA, Sharon, MA (US); Bertrand LE BOURDONNEC, Northborough, MA (US); Matthew LUCAS, Lexington, MA (US); Vanessa KURIA, Somerville, MA (US); Madeline MACDONNELL, Brighton, MA (US); Byron DELABARRE, Arlington, MA (US)

(21) Appl. No.: 18/007,807

(22) PCT Filed: Jun. 3, 2021
(86) PCT No.: PCT/US2021/035698

§ 371 Date: Dec. 2, 2022

Related U.S. Application Data
(60) Provisional application No. 63/034,306, filed on Jun. 3, 2020.

(51) Int. Cl. C07D 471/04; A61K 31/5377

(52) U.S. Cl. CPC C07D 471/04; A61K 31/5377

(57) ABSTRACT

Disclosed are compounds useful in the treatment of neurological disorders. The compounds described herein, alone or in combination with other pharmaceutically active agents, can be used for treating or preventing neurological diseases.

### FIELD OF THE INVENTION

The invention relates to bicyclic heteroarenes and their use for therapeutic treatment of neurological disorders in patients, such as human patients.

### BACKGROUND

An incomplete understanding of the molecular perturbations that cause disease, as well as a limited arsenal of robust model systems, has contributed to a failure to generate successful disease-modifying therapies against common and progressive neurological disorders, such as ALS and FTD. Progress is being made on many fronts to find agents that can arrest the progress of these disorders. However, the present therapies for most, if not all, of these diseases provide very little relief. Accordingly, a need exists to develop therapies that can alter the course of neurodegenerative diseases. More generally, a need exists for better methods and compositions for the treatment of neurodegenerative diseases in order to improve the quality of the lives of those afflicted by such diseases.

### SUMMARY

TDP-43 is a nuclear DNA/RNA binding protein involved in RNA splicing. Under pathological cell stress, TDP-43 translocates to the cytoplasm and aggregates into stress granules and related protein inclusions. These phenotypes are hallmarks of degenerating motor neurons and are found in 97% of all ALS cases. The highly penetrant nature of this pathology indicates that TDP-43 is broadly involved in both familial and sporadic ALS. Additionally, TDP-43 mutations that promote aggregation are linked to higher risk of developing ALS, suggesting protein misfolding and aggregation act as drivers of toxicity. TDP-43 toxicity can be recapitulated in yeast models, where the protein induces a viability deficit and localizes to stress granules.

In an aspect, the invention features a compound of Formula I:

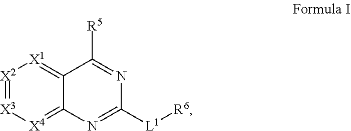

or a pharmaceutically acceptable salt thereof,
where
X¹ is N or CR¹;
X² is N or CR²;
X³ is N or CR³;
X⁴ is N or CR⁴;
wherein one and only one of X¹, X², X³, and X⁴ is N;
R⁵ is

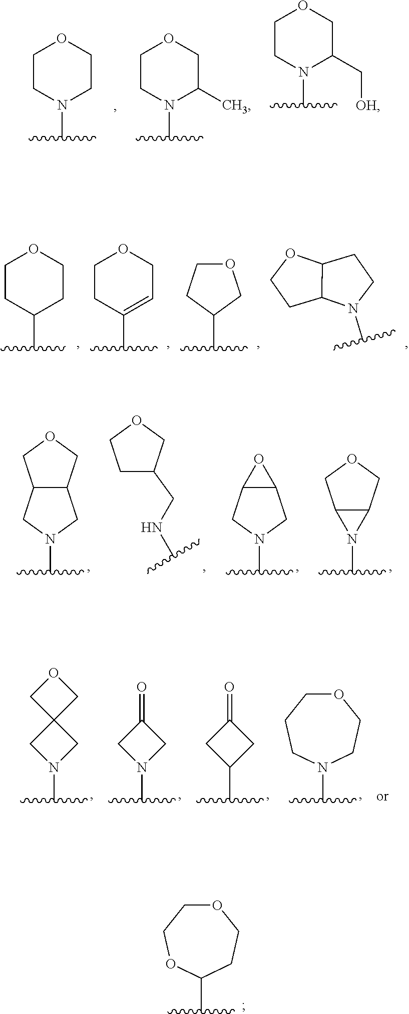

L¹ is

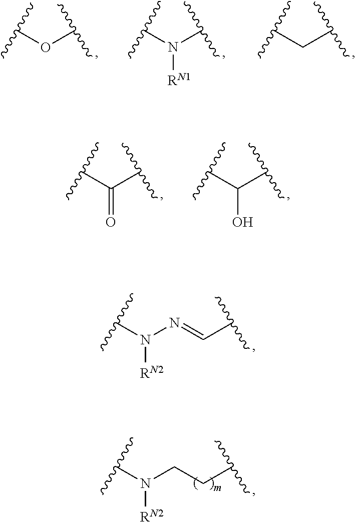

optionally substituted C₁₋₉ heteroarylene having at least one 5-membered ring, or optionally substituted non-aromatic C₁₋₉ heterocyclylene; and R⁶ is optionally substituted C₁₋₆ alkyl, optionally substituted C₆₋₁₀ aryl, optionally substituted C₃₋₁₀ carbocyclyl, optionally substituted C₁₋₉ heteroaryl, optionally substituted C₁₋₉ heterocyclyl, or optionally substituted —C₁₋₆ alkylene-C₁₋₉ heterocyclyl; or L¹ and R⁶ combine to form an optionally substituted C₂₋₉ oxyheteroaryl, optionally substituted pyrimidin-4-yl, or optionally substituted pyrid-2-yl;
R¹ is hydrogen, halogen, or optionally substituted C₁₋₆ alkyl;
R² is hydrogen;
R³ is hydrogen, halogen, optionally substituted C₁₋₆ alkyl, or

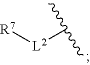

R⁴ is hydrogen, halogen, or optionally substituted C₁₋₆ alkyl;
L² is absent,

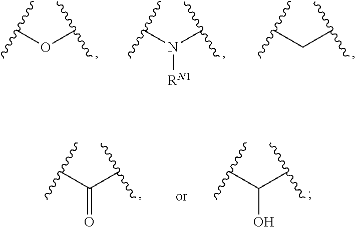

R⁷ is optionally substituted C₆₋₁₀ aryl, optionally substituted C₃₋₁₀ carbocyclyl, optionally substituted C₁₋₉ heteroaryl, or optionally substituted C₁₋₉ heterocyclyl;
each of R^{N1}, R^{N2} and R^{N3} is, independently, hydrogen or optionally substituted C₁₋₆ alkyl; and
m is 0, 1, 2, or 3.

In some embodiments, R⁵ is

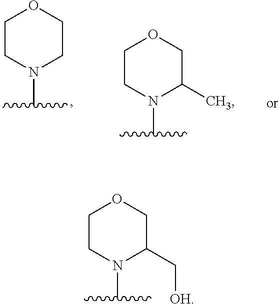 [SMILES: *N1CCOCC1 ; *N1CCOCC1C ; *N1CCOCC1CO]

In some embodiments, R⁵ is

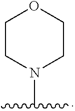 [SMILES: *N1CCOCC1]

In some embodiments, the compound has the structure of Formula Ia:

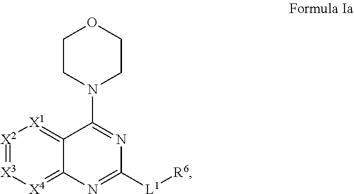

or a pharmaceutically acceptable salt thereof.

In some embodiments, X¹ is N. In some embodiments, X² is N. In some embodiments, X³ is N. In some embodiments, X⁴ is N.

In some embodiments, the compound has the structure of Formula II:

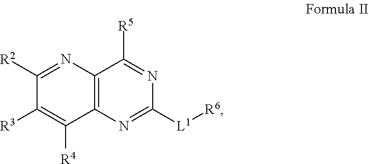

or a pharmaceutically acceptable salt thereof.

In some embodiments, R² is halogen or optionally substituted C₁₋₆ alkyl.

In some embodiments, the compound has the structure of Formula IIa:

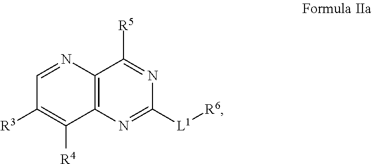

or a pharmaceutically acceptable salt thereof.

In some embodiments, R⁴ is halogen or optionally substituted C₁₋₆ alkyl.

In some embodiments, the compound has the structure of Formula IIb:

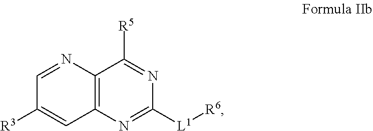

or a pharmaceutically acceptable salt thereof.

In some embodiments, the compound has the structure of Formula IIc:

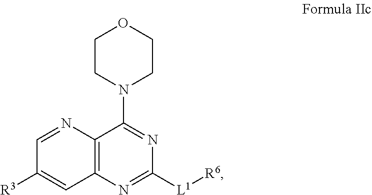

or a pharmaceutically acceptable salt thereof.

In some embodiments, R³ is halogen or optionally substituted C₁₋₆ alkyl.

In some embodiments, R³ is Br.

In some embodiments, R³ is

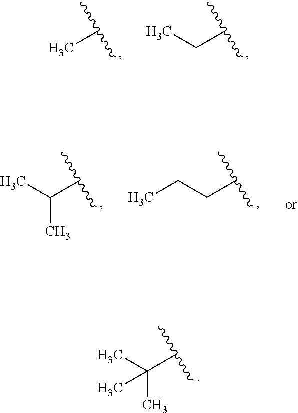

In some embodiments, R³ is

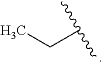 [SMILES: *CC]

In some embodiments, R³ is

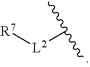

In some embodiments, L² is absent.

In some embodiments, L² is

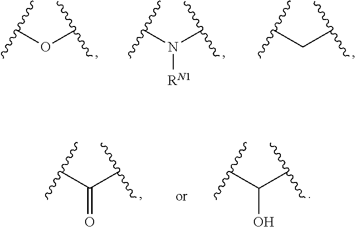

In some embodiments, R^{N1} is hydrogen or

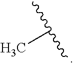

In some embodiments, R^{N1} is hydrogen.

In some embodiments, L² is

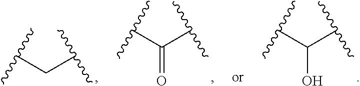

In some embodiments, R⁷ is optionally substituted C₆₋₁₀ aryl.

In some embodiments, R⁷ is optionally substituted C₃₋₁₀ carbocyclyl, optionally substituted C₁₋₉ heteroaryl, or optionally substituted C₁₋₉ heterocyclyl.

In some embodiments, R⁷ is optionally substituted C₃₋₁₀ carbocyclyl.

In some embodiments, R⁷ is

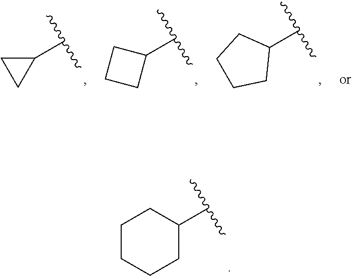

In some embodiments, R⁷ is

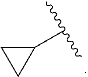 [SMILES: *C1CC1]

In some embodiments, R⁷ is optionally substituted C₁₋₉ heteroaryl or optionally substituted C₁₋₉ heterocyclyl.

In some embodiments, R⁷ is optionally substituted C₁₋₉ heteroaryl.

In some embodiments, R⁷ is

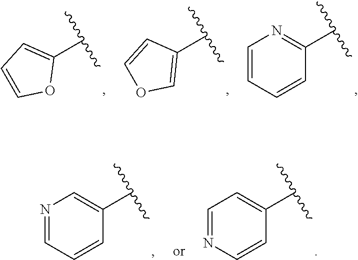

In some embodiments, R⁷ is

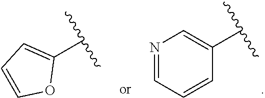 [SMILES: *c1ccco1 ; *c1cccnc1]

In some embodiments, R⁷ is or optionally substituted C₁₋₉ heterocyclyl.

In some embodiments, R⁷ is

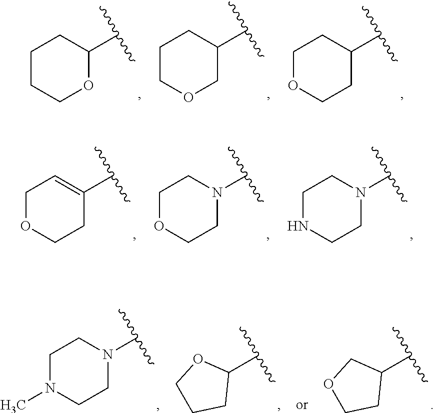

In some embodiments, R⁷ is

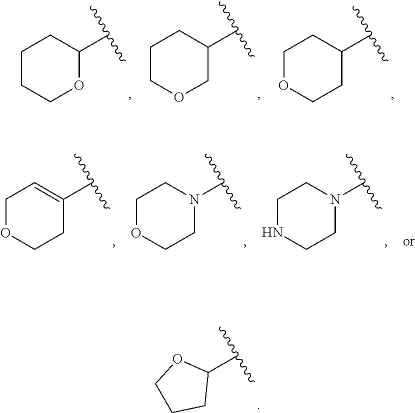

In some embodiments, R⁷ is

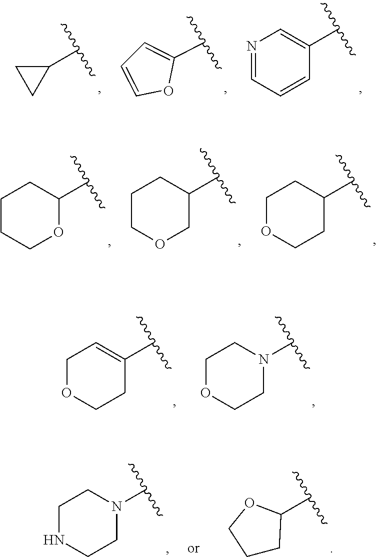

In some embodiments, the compound has the structure of Formula IId:

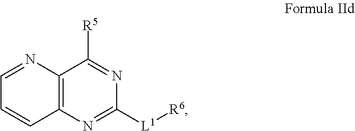

or a pharmaceutically acceptable salt thereof.

In some embodiments, the compound has the structure of Formula IIe:

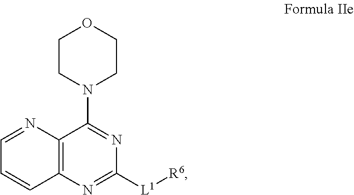

or a pharmaceutically acceptable salt thereof.

In some embodiments, the compound has the structure of Formula IIf:

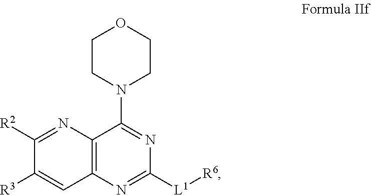

or a pharmaceutically acceptable salt thereof.

In some embodiments, the compound has the structure of Formula IIg:

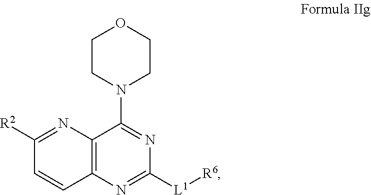

or a pharmaceutically acceptable salt thereof.

In some embodiments, R² is optionally substituted C₁₋₉ heterocyclyl.

In some embodiments, R² is

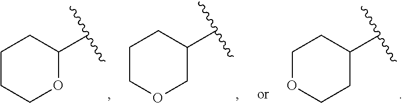

In some embodiments, R² is

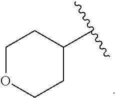 [SMILES: *C1CCOCC1]

In some embodiments, the compound has the structure of Formula III:

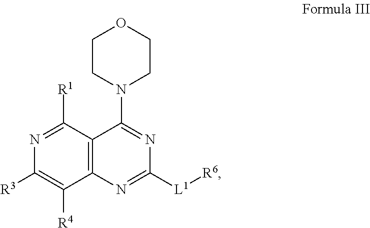

or a pharmaceutically acceptable salt thereof.

In some embodiments, the compound has the structure of Formula IIIa:

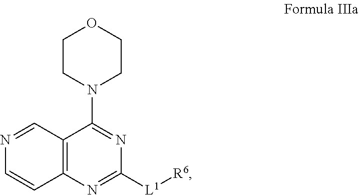

or a pharmaceutically acceptable salt thereof.

In some embodiments, the compound has the structure of Formula IV:

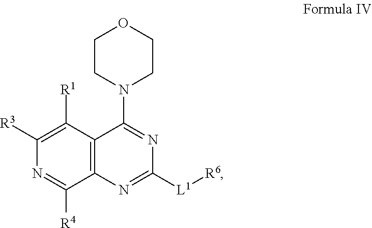

or a pharmaceutically acceptable salt thereof.

In some embodiments, the compound has the structure of Formula IVa:

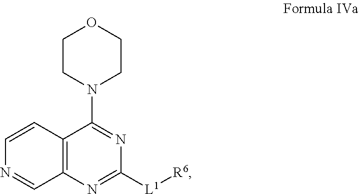

or a pharmaceutically acceptable salt thereof.

In some embodiments, the compound has the structure of Formula V:

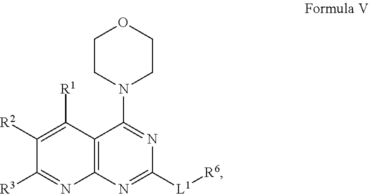

or a pharmaceutically acceptable salt thereof.

In some embodiments, the compound has the structure of Formula Va:

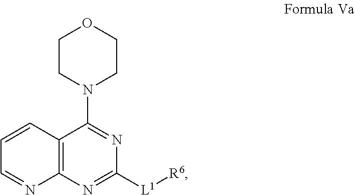

or a pharmaceutically acceptable salt thereof.

In some embodiments, L¹ is

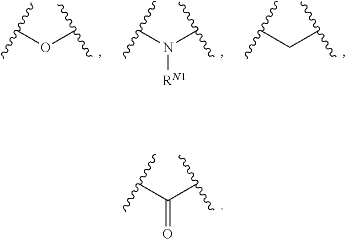

In some embodiments, L¹ is

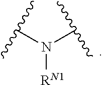

In some embodiments, R^{N1} is hydrogen or

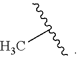

In some embodiments, R^{N1} is hydrogen.

In some embodiments, L¹ is

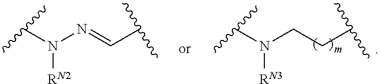

In some embodiments, L¹ is

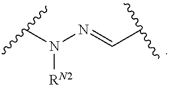

In some embodiments, R^{N2} is hydrogen or

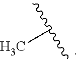

In some embodiments, R^{N2} is hydrogen.

In some embodiments, L¹ is

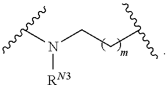

In some embodiments, m is 1.

In some embodiments, R^{N3} is hydrogen or

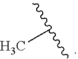

In some embodiments, R^{N3} is hydrogen.

In some embodiments, L¹ is optionally substituted C₁₋₉ heteroarylene having at least one 5-membered ring or non-aromatic optionally substituted C₁₋₉ heterocyclylene.

In some embodiments, L¹ is optionally substituted C₁₋₉ heteroarylene having at least one 5-membered ring.

In some embodiments, L¹ is optionally substituted pyrazole-diyl.

In some embodiments, L¹ is optionally substituted monocyclic 5-membered C₁₋₉ heteroarylene.

In some embodiments, L¹ is optionally substituted pyrazole-diyl or optionally substituted triazole-diyl.

In some embodiments, L¹ is

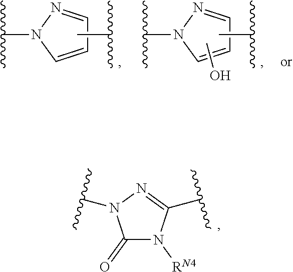

where R^{N4} is hydrogen or optionally substituted C₁₋₆ alkyl.

In some embodiments, L¹ is

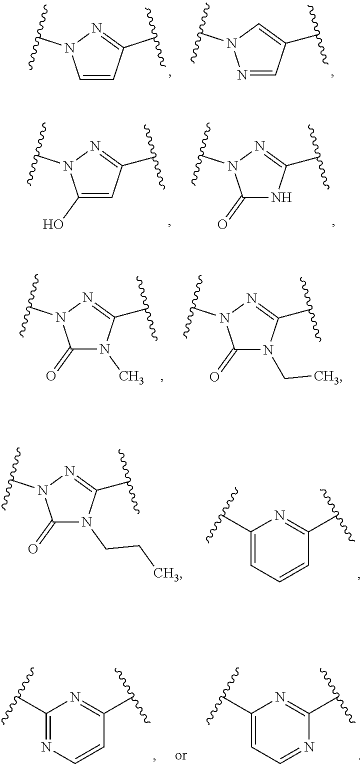

In some embodiments, L¹ is

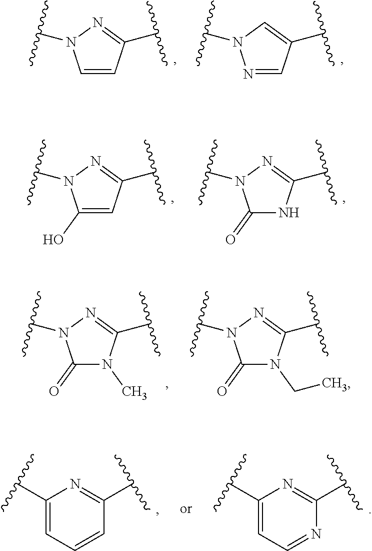

In some embodiments, L¹ is optionally substituted non-aromatic C₁₋₉ heterocyclylene.

In some embodiments, L¹ is optionally substituted non-aromatic C₁₋₅ heterocyclylene.

In some embodiments, L¹ is optionally substituted non-aromatic C₁₋₄ heterocyclylene.

In some embodiments, L¹ is optionally substituted 5-membered non-aromatic C₁₋₅ heterocyclylene.

In some embodiments, L¹ is

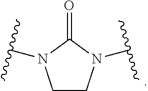 [SMILES: *N1CCN(*)C1=O]

In some embodiments, R⁶ is optionally substituted C₁₋₆ alkyl.

In some embodiments, R⁶ is

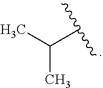 [SMILES: *C(C)C]

In some embodiments, R⁶ is optionally substituted C₆₋₁₀ aryl, optionally substituted C₃₋₁₀ carbocyclyl, optionally substituted C₁₋₉ heteroaryl, optionally substituted C₁₋₉ heterocyclyl, or optionally substituted —C₁₋₆ alkylene-C₁₋₉ heterocyclyl.

In some embodiments, R⁶ is optionally substituted C₆₋₁₀ aryl.

In some embodiments, R⁶ is

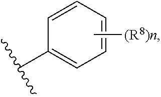

where n is 0, 1, 2, 3, 4, or 5; and each R⁸ is, independently, halogen or optionally substituted C₁₋₆ alkyl.

In some embodiments, each R⁸ is, independently, F or

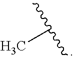

In some embodiments, n is 0 or 1.

In some embodiments, R⁶ is

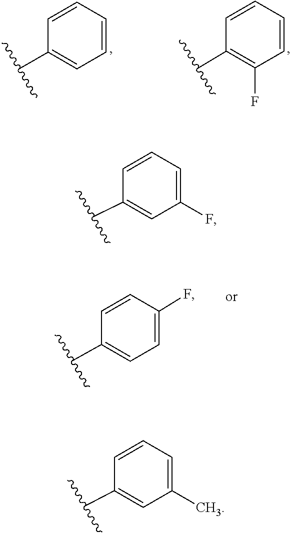 [SMILES: *c1ccccc1 ; *c1ccccc1F ; *c1cccc(F)c1 ; *c1ccc(F)cc1 ; *c1cccc(C)c1]

In some embodiments, R⁶ is optionally substituted C₃₋₁₀ carbocyclyl.

In some embodiments, R⁶ is

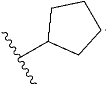 [SMILES: *C1CCCC1]

In some embodiments, R⁶ is optionally substituted C₁₋₉ heteroaryl.

In some embodiments, R⁶ is optionally substituted monocyclic C₁₋₉ heteroaryl.

In some embodiments, R⁶ is

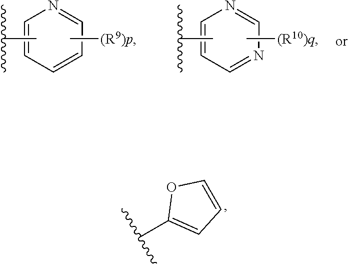

where p is 0, 1, 2, or 3; q is 0, 1, or 2; each R⁹ is, independently, halogen or optionally substituted C₁₋₆ alkyl; and each R¹⁰ is, independently, halogen or optionally substituted C₁₋₆ alkyl.

In some embodiments, p is 0.

In some embodiments, q is 0.

In some embodiments, R⁶ is

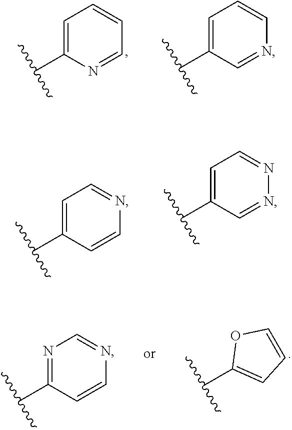 [SMILES: *c1ccccn1 ; *c1cccnc1 ; *c1ccncc1 ; *c1ccnnc1 ; *c1ccncn1 ; *c1ccco1]

In some embodiments, R⁶ is optionally substituted polycyclic C₁₋₉ heteroaryl.

In some embodiments, R⁶ is

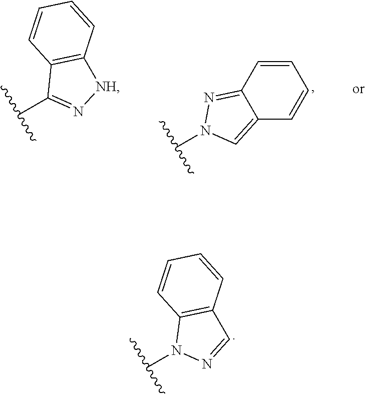 [SMILES: *c1n[nH]c2ccccc12 ; *n1cc2ccccc2n1 ; *n1ncc2ccccc21]

In some embodiments, R⁶ is optionally substituted C₁₋₉ heterocyclyl.

In some embodiments, R⁶ is optionally substituted monocyclic C₁₋₉ heterocyclyl.

In some embodiments, R⁶ is

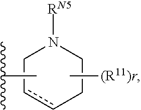

where [symbol] represents a single bond or a double bond; r is 0, 1, 2, 3, 4, 5, or 6; each R¹¹ is, independently, halogen or optionally substituted C₁₋₆ alkyl; and R^{N5} is hydrogen, optionally substituted C₁₋₆ alkyl, or optionally substituted C₁₋₆ heteroalkyl.

In some embodiments, r is 0, 1, or 2.

In some embodiments, R^{N5} is hydrogen or

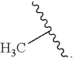

In some embodiments, R⁶ is

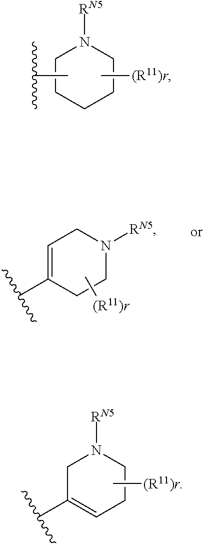

In some embodiments, R⁶ is

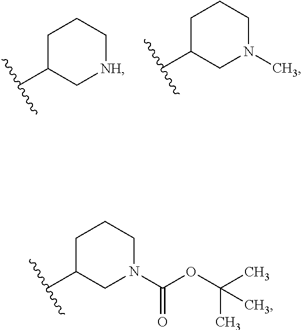 [SMILES: *C1CCCNC1 ; *C1CCCN(C)C1 ; *C1CCCN(C(=O)OC(C)(C)C)C1]

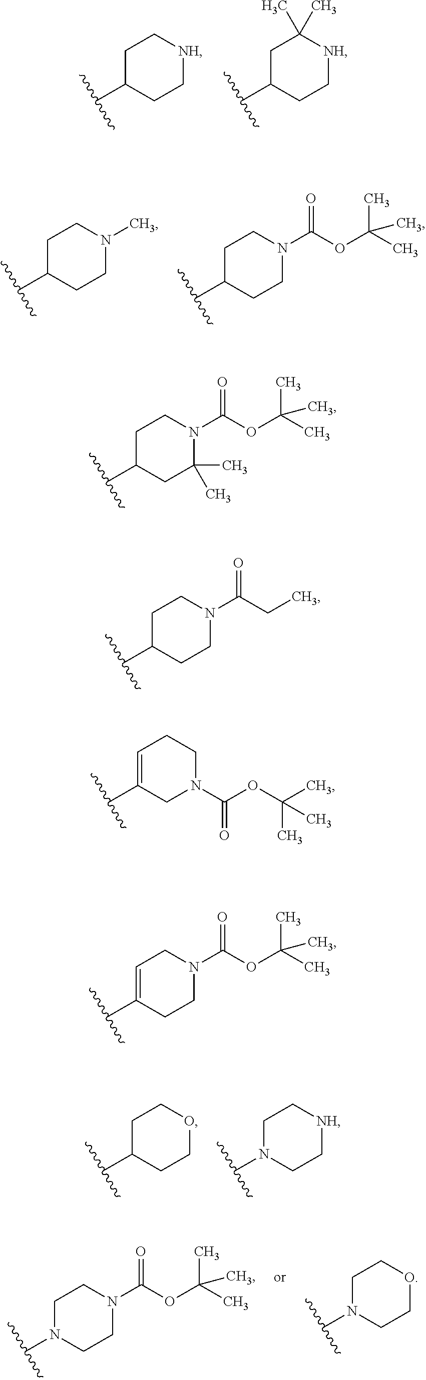

In some embodiments, R⁶ is optionally substituted —C₁₋₆ alkylene-C₁₋₉ heterocyclyl.

In some embodiments, R⁶ is

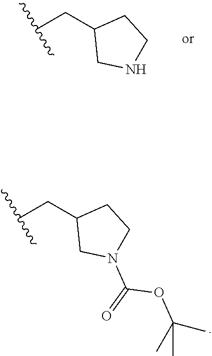 [SMILES: *CC1CCNC1 ; *CC1CCN(C(=O)OC(C)(C)C)C1]

In some embodiments, L¹ and R⁶ combine to form optionally substituted pyrimidin-4-yl. In some embodiments, the optionally substituted pyrimidin-4-yl is a pyrimidin-4-yl substituted at position 2.

In some embodiments, L¹ and R⁶ combine to form optionally substituted pyrid-2-yl. In some embodiments, the optionally substituted pyrid-2-yl is a pyrid-2-yl substituted at position 5.

In some embodiments, the compound has the structure of any one of compounds 1-88 in Table 1.

In an aspect, the disclosure features compounds 1-88 in Table 1.

Non-limiting examples of the compounds of the invention include the compounds in Table 1.

TABLE 1
Compounds of the Invention

| # | Structure |
|---|---|
| 1 | 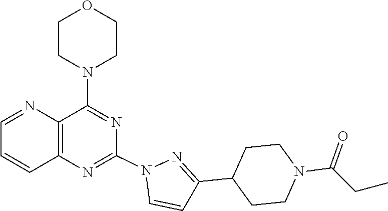 [SMILES: CCC(=O)N1CCC(c2ccn(-c3nc(N4CCOCC4)c4ncccc4n3)n2)CC1] |
| 2 | 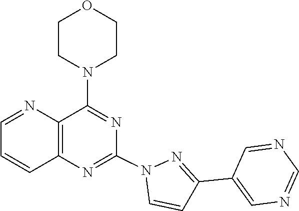 [SMILES: c1cnc2c(N3CCOCC3)nc(-n3ccc(-c4cncnc4)n3)nc2c1] |
| 3 | 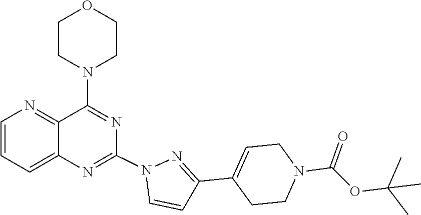 [SMILES: CC(C)(C)OC(=O)N1CC=C(c2ccn(-c3nc(N4CCOCC4)c4ncccc4n3)n2)CC1] |
| 4 | 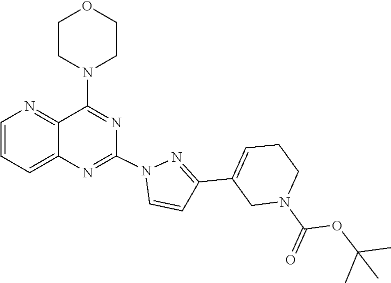 [SMILES: CC(C)(C)OC(=O)N1CCC=C(c2ccn(-c3nc(N4CCOCC4)c4ncccc4n3)n2)C1] |
| 5 | 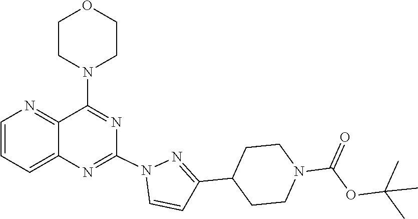 [SMILES: CC(C)(C)OC(=O)N1CCC(c2ccn(-c3nc(N4CCOCC4)c4ncccc4n3)n2)CC1] |
| 6 | 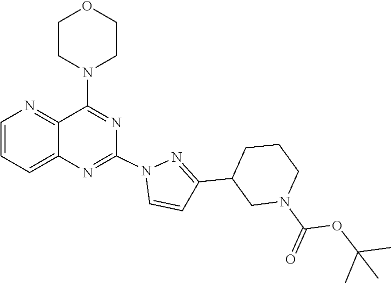 [SMILES: CC(C)(C)OC(=O)N1CCCC(c2ccn(-c3nc(N4CCOCC4)c4ncccc4n3)n2)C1] |
| 7 | 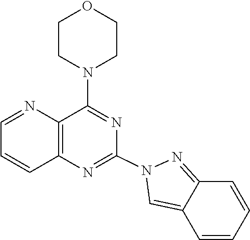 [SMILES: c1ccc2nn(-c3nc(N4CCOCC4)c4ncccc4n3)cc2c1] |
| 8 | 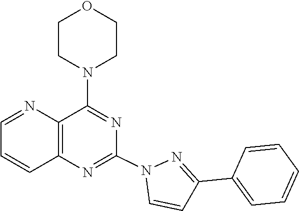 [SMILES: c1ccc(-c2ccn(-c3nc(N4CCOCC4)c4ncccc4n3)n2)cc1] |
| 9 | 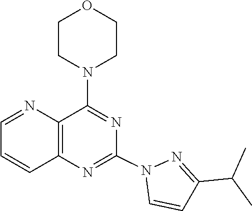 [SMILES: CC(C)c1ccn(-c2nc(N3CCOCC3)c3ncccc3n2)n1] |
| 10 | 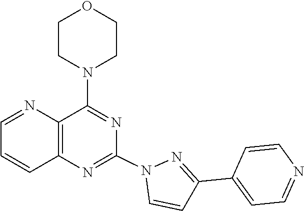 [SMILES: c1cnc2c(N3CCOCC3)nc(-n3ccc(-c4ccncc4)n3)nc2c1] |
| 11 | 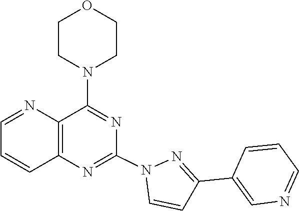 [SMILES: c1cncc(-c2ccn(-c3nc(N4CCOCC4)c4ncccc4n3)n2)c1] |
| 12 | 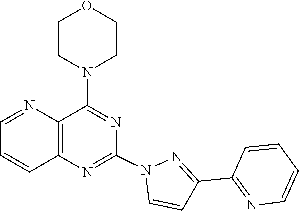 [SMILES: c1ccc(-c2ccn(-c3nc(N4CCOCC4)c4ncccc4n3)n2)nc1] |
| 13 | 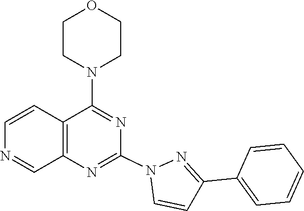 [SMILES: c1ccc(-c2ccn(-c3nc(N4CCOCC4)c4ccncc4n3)n2)cc1] |
| 14 | 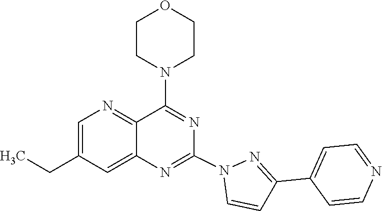 [SMILES: CCc1cnc2c(N3CCOCC3)nc(-n3ccc(-c4ccncc4)n3)nc2c1] |
| 15 | 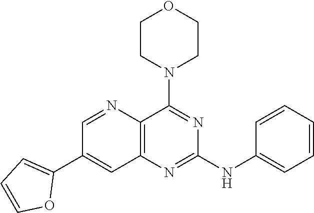 [SMILES: c1ccc(Nc2nc(N3CCOCC3)c3ncc(-c4ccco4)cc3n2)cc1] |
| 16 | 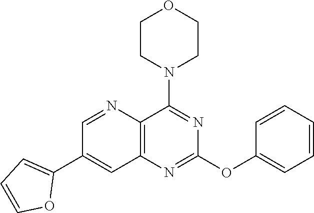 [SMILES: c1ccc(Oc2nc(N3CCOCC3)c3ncc(-c4ccco4)cc3n2)cc1] |
| 17 | 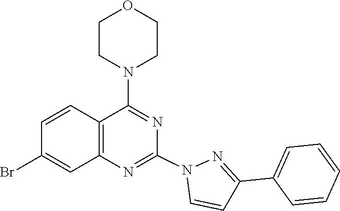 [SMILES: Brc1ccc2c(N3CCOCC3)nc(-n3ccc(-c4ccccc4)n3)nc2c1] |
| 18 | 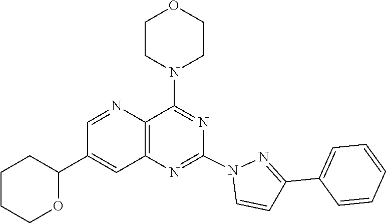 [SMILES: c1ccc(-c2ccn(-c3nc(N4CCOCC4)c4ncc(C5CCCCO5)cc4n3)n2)cc1] |
| 19 | 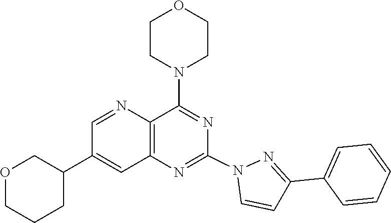 [SMILES: c1ccc(-c2ccn(-c3nc(N4CCOCC4)c4ncc(C5CCCOC5)cc4n3)n2)cc1] |
| 20 | 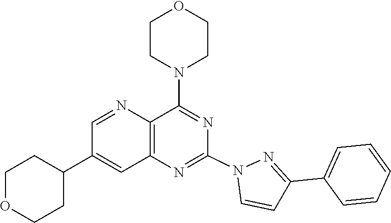 [SMILES: c1ccc(-c2ccn(-c3nc(N4CCOCC4)c4ncc(C5CCOCC5)cc4n3)n2)cc1] |
| 21 | 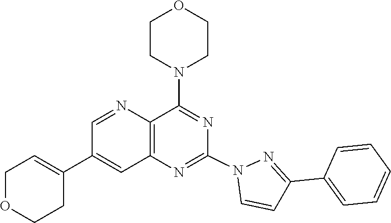 [SMILES: C1=C(c2cnc3c(N4CCOCC4)nc(-n4ccc(-c5ccccc5)n4)nc3c2)CCOC1] |
| 22 | 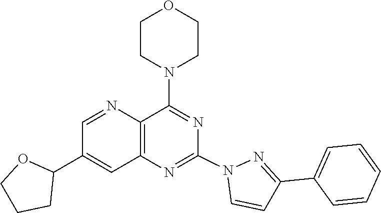 [SMILES: c1ccc(-c2ccn(-c3nc(N4CCOCC4)c4ncc(C5CCCO5)cc4n3)n2)cc1] |
| 23 | 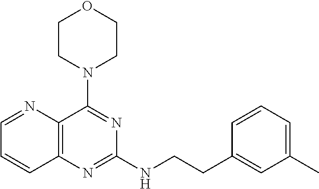 [SMILES: Cc1cccc(CCNc2nc(N3CCOCC3)c3ncccc3n2)c1] |
| 24 | 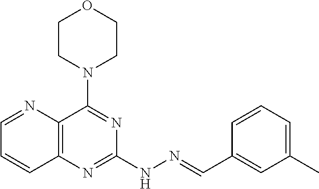 [SMILES: Cc1cccc(/C=N/Nc2nc(N3CCOCC3)c3ncccc3n2)c1] |
| 25 | 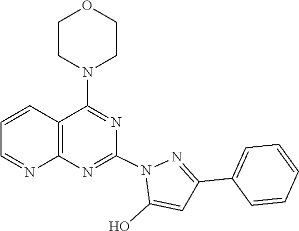 [SMILES: Oc1cc(-c2ccccc2)nn1-c1nc(N2CCOCC2)c2cccnc2n1] |
| 26 | 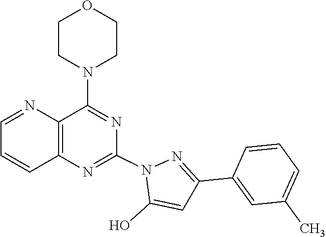 [SMILES: Cc1cccc(-c2cc(O)n(-c3nc(N4CCOCC4)c4ncccc4n3)n2)c1] |
| 27 | 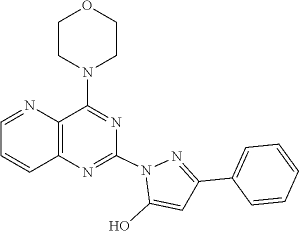 [SMILES: Oc1cc(-c2ccccc2)nn1-c1nc(N2CCOCC2)c2ncccc2n1] |
| 28 | 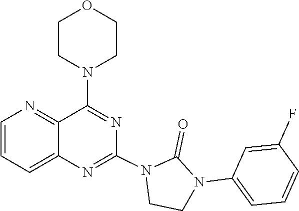 [SMILES: O=C1N(c2cccc(F)c2)CCN1c1nc(N2CCOCC2)c2ncccc2n1] |
| 29 | 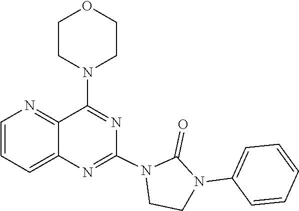 [SMILES: O=C1N(c2ccccc2)CCN1c1nc(N2CCOCC2)c2ncccc2n1] |
| 30 | 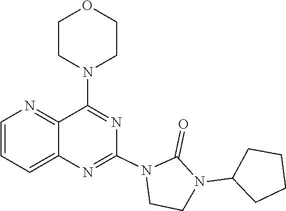 [SMILES: O=C1N(c2nc(N3CCOCC3)c3ncccc3n2)CCN1C1CCCC1] |
| 31 | 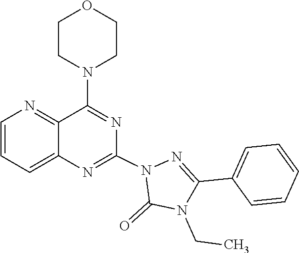 [SMILES: CCn1c(-c2ccccc2)nn(-c2nc(N3CCOCC3)c3ncccc3n2)c1=O] |
| 32 | 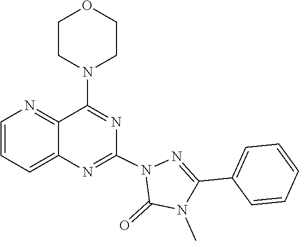 [SMILES: Cn1c(-c2ccccc2)nn(-c2nc(N3CCOCC3)c3ncccc3n2)c1=O] |
| 33 | 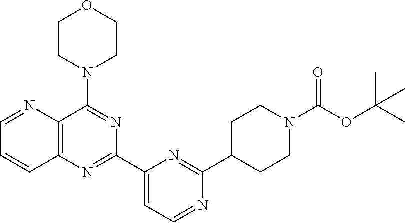 [SMILES: CC(C)(C)OC(=O)N1CCC(c2nccc(-c3nc(N4CCOCC4)c4ncccc4n3)n2)CC1] |
| 34 | 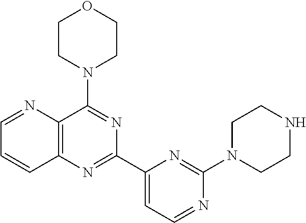 [SMILES: c1cnc2c(N3CCOCC3)nc(-c3ccnc(N4CCNCC4)n3)nc2c1] |
| 35 | 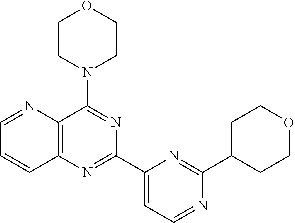 [SMILES: c1cnc2c(N3CCOCC3)nc(-c3ccnc(C4CCOCC4)n3)nc2c1] |
| 36 | 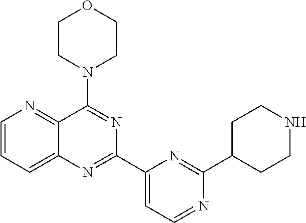 [SMILES: c1cnc2c(N3CCOCC3)nc(-c3ccnc(C4CCNCC4)n3)nc2c1] |
| 37 | 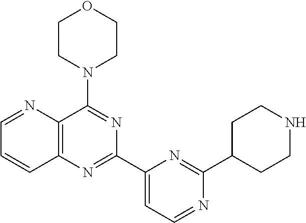 [SMILES: c1cnc2c(N3CCOCC3)nc(-c3ccnc(C4CCNCC4)n3)nc2c1] |
| 38 | 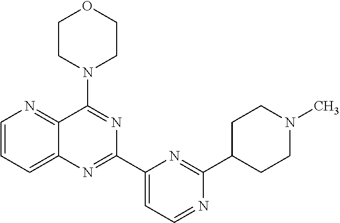 [SMILES: CN1CCC(c2nccc(-c3nc(N4CCOCC4)c4ncccc4n3)n2)CC1] |
| 39 | 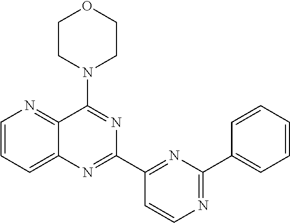 [SMILES: c1ccc(-c2nccc(-c3nc(N4CCOCC4)c4ncccc4n3)n2)cc1] |
| 40 | 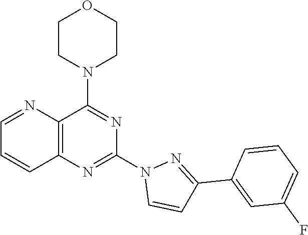 [SMILES: Fc1cccc(-c2ccn(-c3nc(N4CCOCC4)c4ncccc4n3)n2)c1] |
| 41 | 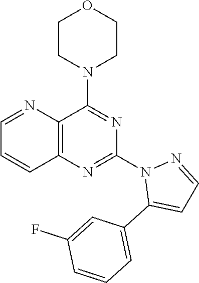 [SMILES: Fc1cccc(-c2ccnn2-c2nc(N3CCOCC3)c3ncccc3n2)c1] |
| 42 | 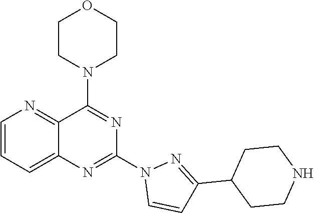 [SMILES: c1cnc2c(N3CCOCC3)nc(-n3ccc(C4CCNCC4)n3)nc2c1] |
| 43 | 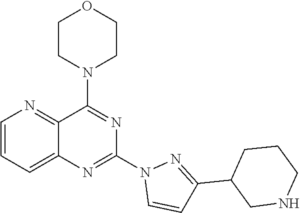 [SMILES: c1cnc2c(N3CCOCC3)nc(-n3ccc(C4CCCNC4)n3)nc2c1] |
| 44 | 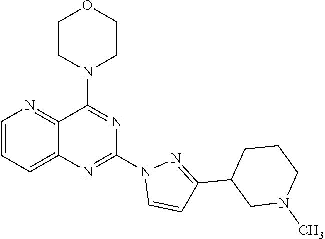 [SMILES: CN1CCCC(c2ccn(-c3nc(N4CCOCC4)c4ncccc4n3)n2)C1] |
| 45 | 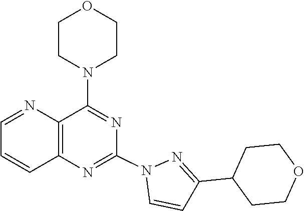 [SMILES: c1cnc2c(N3CCOCC3)nc(-n3ccc(C4CCOCC4)n3)nc2c1] |
| 46 | 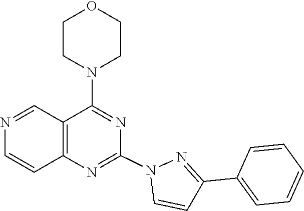 [SMILES: c1ccc(-c2ccn(-c3nc(N4CCOCC4)c4cnccc4n3)n2)cc1] |
| 47 | 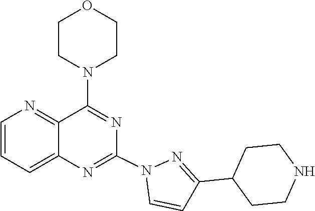 [SMILES: c1cnc2c(N3CCOCC3)nc(-n3ccc(C4CCNCC4)n3)nc2c1] |
| 48 | 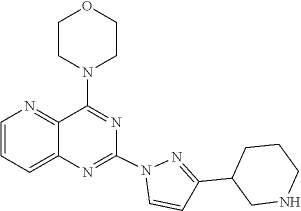 [SMILES: c1cnc2c(N3CCOCC3)nc(-n3ccc(C4CCCNC4)n3)nc2c1] |
| 49 | 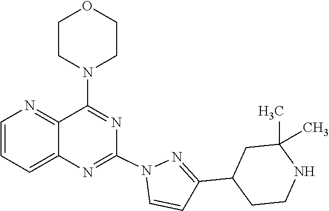 [SMILES: CC1(C)CC(c2ccn(-c3nc(N4CCOCC4)c4ncccc4n3)n2)CCN1] |
| 50 | 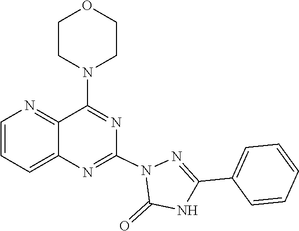 [SMILES: O=c1[nH]c(-c2ccccc2)nn1-c1nc(N2CCOCC2)c2ncccc2n1] |
| 51 | 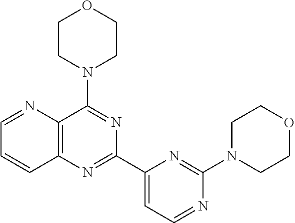 [SMILES: c1cnc2c(N3CCOCC3)nc(-c3ccnc(N4CCOCC4)n3)nc2c1] |
| 52 | 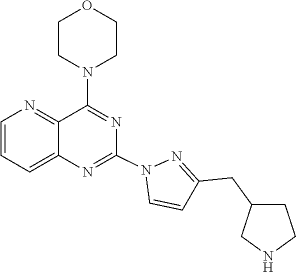 [SMILES: c1cnc2c(N3CCOCC3)nc(-n3ccc(CC4CCNC4)n3)nc2c1] |
| 53 | 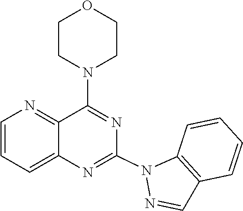 [SMILES: c1ccc2c(c1)cnn2-c1nc(N2CCOCC2)c2ncccc2n1] |
| 54 | 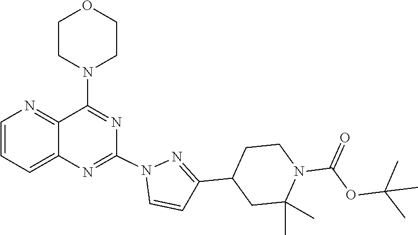 [SMILES: CC(C)(C)OC(=O)N1CCC(c2ccn(-c3nc(N4CCOCC4)c4ncccc4n3)n2)CC1(C)C] |
| 55 | 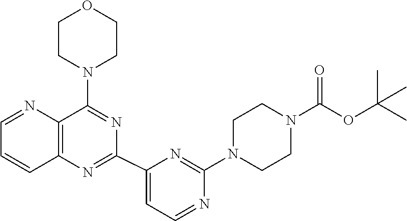 [SMILES: CC(C)(C)OC(=O)N1CCN(c2nccc(-c3nc(N4CCOCC4)c4ncccc4n3)n2)CC1] |
| 56 | 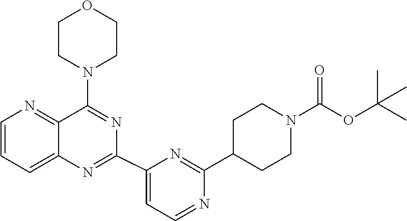 [SMILES: CC(C)(C)OC(=O)N1CCC(c2nccc(-c3nc(N4CCOCC4)c4ncccc4n3)n2)CC1] |
| 57 | 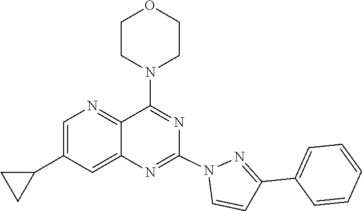 [SMILES: c1ccc(-c2ccn(-c3nc(N4CCOCC4)c4ncc(C5CC5)cc4n3)n2)cc1] |
| 58 | 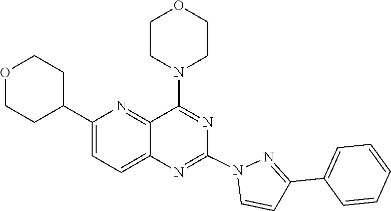 [SMILES: c1ccc(-c2ccn(-c3nc(N4CCOCC4)c4nc(C5CCOCC5)ccc4n3)n2)cc1] |
| 59 | 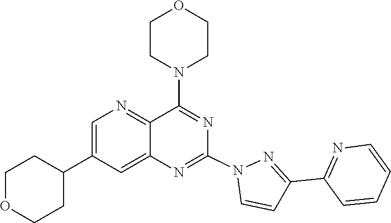 [SMILES: c1ccc(-c2ccn(-c3nc(N4CCOCC4)c4ncc(C5CCOCC5)cc4n3)n2)nc1] |
| 60 | 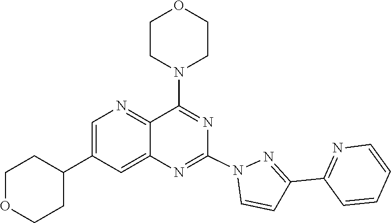 [SMILES: c1ccc(-c2ccn(-c3nc(N4CCOCC4)c4ncc(C5CCOCC5)cc4n3)n2)nc1] |
| 61 | 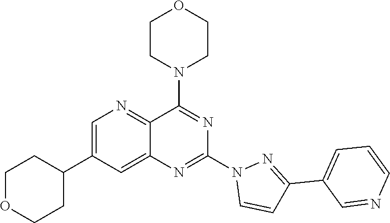 [SMILES: c1cncc(-c2ccn(-c3nc(N4CCOCC4)c4ncc(C5CCOCC5)cc4n3)n2)c1] |
| 62 | 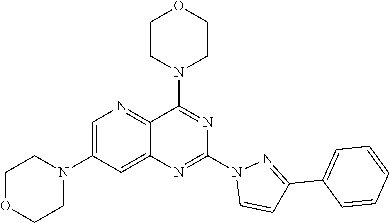 [SMILES: c1ccc(-c2ccn(-c3nc(N4CCOCC4)c4ncc(N5CCOCC5)cc4n3)n2)cc1] |
| 63 | 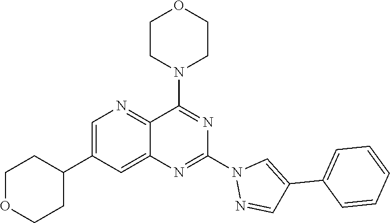 [SMILES: c1ccc(-c2cnn(-c3nc(N4CCOCC4)c4ncc(C5CCOCC5)cc4n3)c2)cc1] |
| 64 | 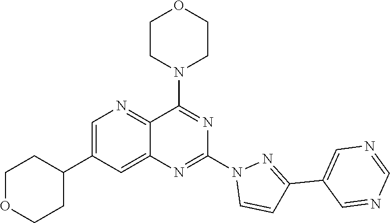 [SMILES: c1ncc(-c2ccn(-c3nc(N4CCOCC4)c4ncc(C5CCOCC5)cc4n3)n2)cn1] |
| 65 | 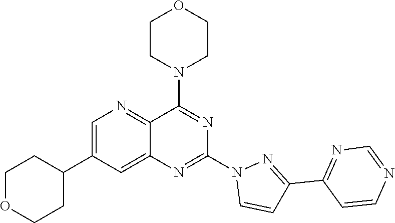 [SMILES: c1cc(-c2ccn(-c3nc(N4CCOCC4)c4ncc(C5CCOCC5)cc4n3)n2)ncn1] |
| 66 | 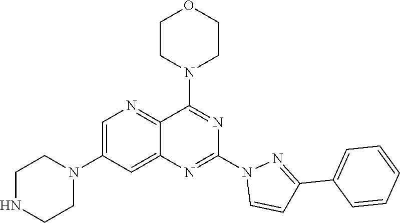 [SMILES: c1ccc(-c2ccn(-c3nc(N4CCOCC4)c4ncc(N5CCNCC5)cc4n3)n2)cc1] |
| 67 | 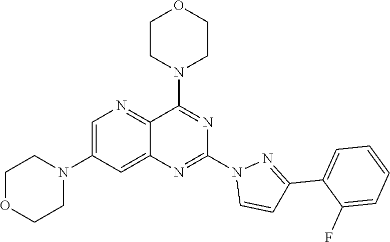 [SMILES: Fc1ccccc1-c1ccn(-c2nc(N3CCOCC3)c3ncc(N4CCOCC4)cc3n2)n1] |
| 68 | 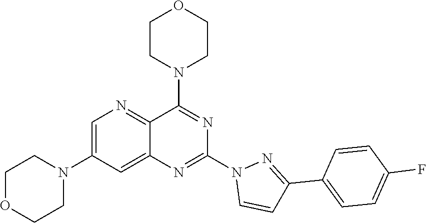 [SMILES: Fc1ccc(-c2ccn(-c3nc(N4CCOCC4)c4ncc(N5CCOCC5)cc4n3)n2)cc1] |
| 69 | 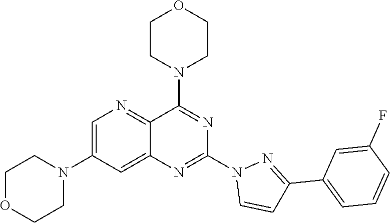 [SMILES: Fc1cccc(-c2ccn(-c3nc(N4CCOCC4)c4ncc(N5CCOCC5)cc4n3)n2)c1] |
| 70 | 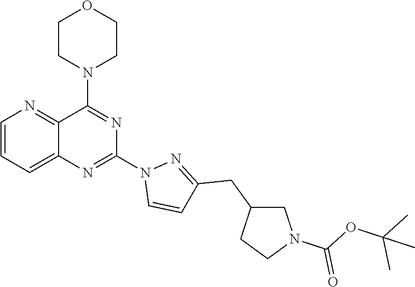 [SMILES: CC(C)(C)OC(=O)N1CCC(Cc2ccn(-c3nc(N4CCOCC4)c4ncccc4n3)n2)C1] |
| 71 | 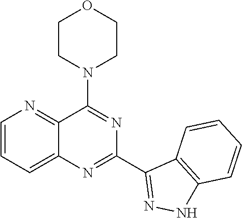 [SMILES: c1cnc2c(N3CCOCC3)nc(-c3n[nH]c4ccccc34)nc2c1] |
| 72 | 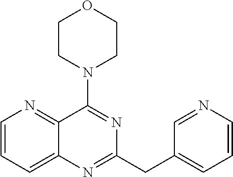 [SMILES: c1cncc(Cc2nc(N3CCOCC3)c3ncccc3n2)c1] |
| 73 | 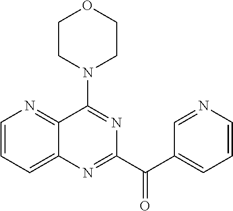 [SMILES: O=C(c1cccnc1)c1nc(N2CCOCC2)c2ncccc2n1] |
| 74 | 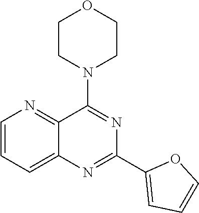 [SMILES: c1coc(-c2nc(N3CCOCC3)c3ncccc3n2)c1] |
| 75 | 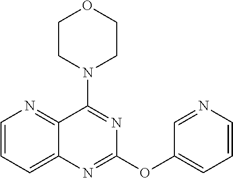 [SMILES: c1cncc(Oc2nc(N3CCOCC3)c3ncccc3n2)c1] |
| 76 | 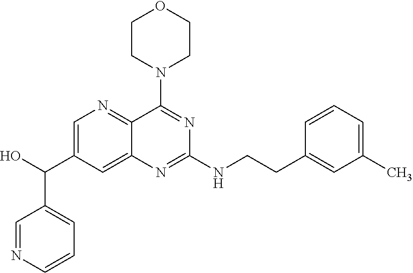 [SMILES: Cc1cccc(CCNc2nc(N3CCOCC3)c3ncc(C(O)c4cccnc4)cc3n2)c1] |
| 77 | 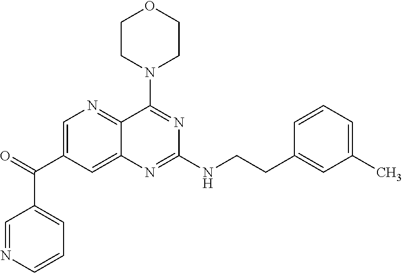 [SMILES: Cc1cccc(CCNc2nc(N3CCOCC3)c3ncc(C(=O)c4cccnc4)cc3n2)c1] |
| 78 | 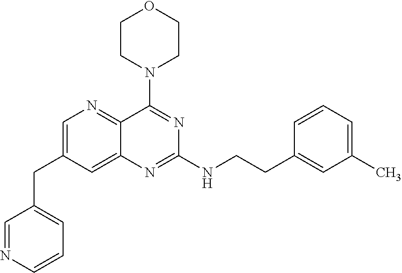 [SMILES: Cc1cccc(CCNc2nc(N3CCOCC3)c3ncc(Cc4cccnc4)cc3n2)c1] |
| 79 | 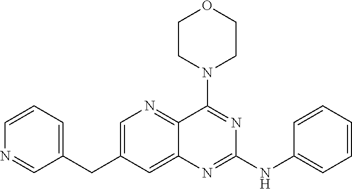 [SMILES: c1ccc(Nc2nc(N3CCOCC3)c3ncc(Cc4cccnc4)cc3n2)cc1] |
| 80 | 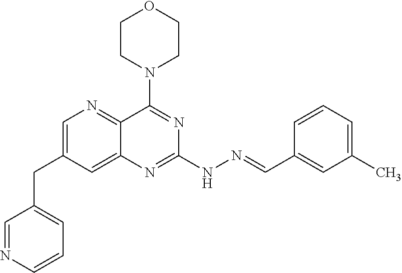 [SMILES: Cc1cccc(/C=N/Nc2nc(N3CCOCC3)c3ncc(Cc4cccnc4)cc3n2)c1] |
| 81 | 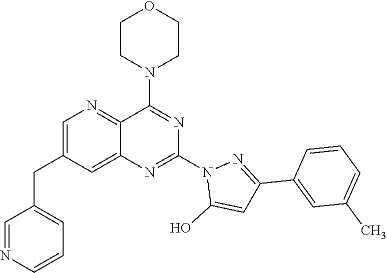 [SMILES: Cc1cccc(-c2cc(O)n(-c3nc(N4CCOCC4)c4ncc(Cc5cccnc5)cc4n3)n2)c1] |
| 82 | 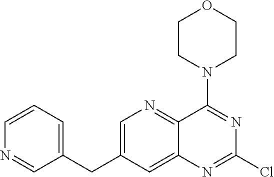 [SMILES: Clc1nc(N2CCOCC2)c2ncc(Cc3cccnc3)cc2n1] |
| 83 | 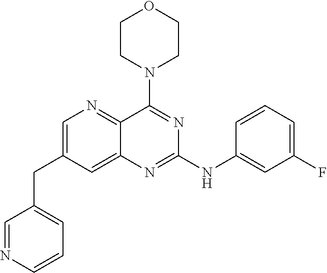 [SMILES: Fc1cccc(Nc2nc(N3CCOCC3)c3ncc(Cc4cccnc4)cc3n2)c1] |
| 84 | 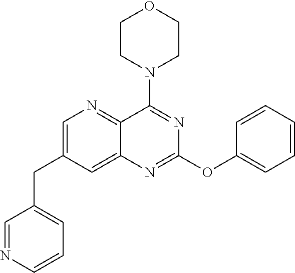 [SMILES: c1ccc(Oc2nc(N3CCOCC3)c3ncc(Cc4cccnc4)cc3n2)cc1] |
| 85 | 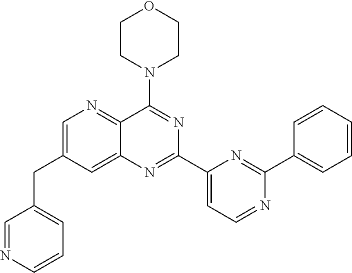 [SMILES: c1ccc(-c2nccc(-c3nc(N4CCOCC4)c4ncc(Cc5cccnc5)cc4n3)n2)cc1] |
| 86 | 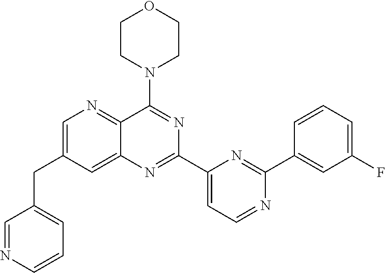 [SMILES: Fc1cccc(-c2nccc(-c3nc(N4CCOCC4)c4ncc(Cc5cccnc5)cc4n3)n2)c1] |
| 87 | 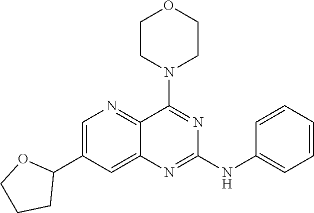 [SMILES: c1ccc(Nc2nc(N3CCOCC3)c3ncc(C4CCCO4)cc3n2)cc1] |
| 88 | 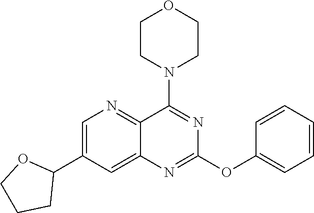 [SMILES: c1ccc(Oc2nc(N3CCOCC3)c3ncc(C4CCCO4)cc3n2)cc1] |

In an aspect, the invention features a pharmaceutical composition comprising any of the foregoing compounds and a pharmaceutically acceptable excipient.

In an aspect, the invention features a method of treating a neurological disorder (e.g., frontotemporal dementia (FTLD-TDP), chronic traumatic encephalopathy, ALS, Alzheimer's disease, limbic-predominant age-related TDP-43 encephalopathy (LATE), or frontotemporal lobar degeneration) in a subject in need thereof. This method includes administering an effective amount of any of the foregoing compounds or pharmaceutical compositions.

In an aspect, the invention features a method of inhibiting toxicity in a cell (e.g., mammalian neural cell) related to a protein (e.g., TDP-43 or C9orf72). This method includes administering an effective amount of any of the foregoing compounds or pharmaceutical compositions.

In an aspect, the invention features a method of treating a TDP-43-associated disorder or C9orf72-associated disorder (e.g., FTLD-TDP, chronic traumatic encephalopathy, ALS, Alzheimer's disease, LATE, or frontotemporal lobar degeneration) in a subject in need thereof. This method includes administering to the subject an effective amount of a compound described herein or a pharmaceutical composition containing one or more compounds described herein.

In some embodiments, the method includes administering to the subject in need thereof an effective amount of the compound of Formula I:

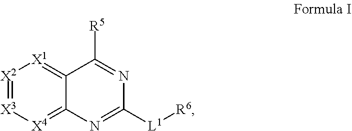

or a pharmaceutically acceptable salt thereof,
where
X¹ is N or CR¹;
X² is N or CR²;
X³ is N or CR³;
X⁴ is N or CR⁴;
R⁵ is

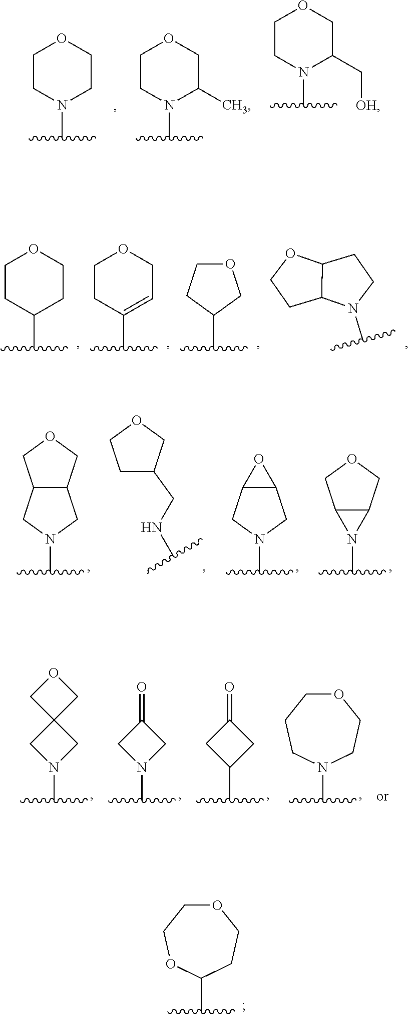

L¹ is absent,

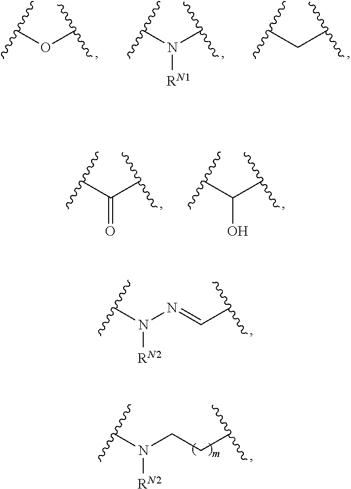

optionally substituted C₁₋₉ heteroarylene, or optionally substituted C₁₋₉ heterocyclylene;
R⁶ is halogen, optionally substituted C₁₋₆ alkyl, optionally substituted C₆₋₁₀ aryl, optionally substituted C₃₋₁₀ carbocyclyl, optionally substituted C₁₋₉ heteroaryl, optionally substituted C₁₋₉ heterocyclyl, or optionally substituted —C₁₋₆ alkylene-C₁₋₉ heterocyclyl,
R¹ is hydrogen, halogen, optionally substituted C₁₋₆ alkyl, or

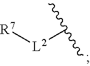

R² is hydrogen, halogen, optionally substituted C₁₋₆ alkyl, or

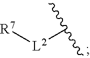

R³ is hydrogen, halogen, optionally substituted C₁₋₆ alkyl, or

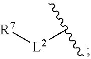

R⁴ is hydrogen, halogen, optionally substituted C₁₋₆ alkyl, or

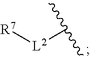

L² is absent,

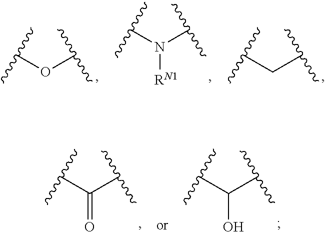

R⁷ is optionally substituted C₆₋₁₀ aryl, optionally substituted C₃₋₁₀ carbocyclyl, optionally substituted C₁₋₉ heteroaryl, or optionally substituted C₁₋₉ heterocyclyl;
each of R^{N1}, R^{N2} and R^{N3} is, independently, hydrogen or optionally substituted C₁₋₆ alkyl;
m is 0, 1, 2, or 3; and
where one of X¹, X², X³, and X⁴ is N;
where if X¹ is N, then X² is CR², X³ is CR³, and X⁴ is CR⁴;
where if X² is N, then X¹ is CR¹, X³ is CR³, and X⁴ is CR⁴;
where if X³ is N, then X¹ is CR¹, X² is CR², and X⁴ is CR⁴; and
where if X⁴ is N, then X¹ is CR¹, X² is CR², and X³ is CR³.

In some embodiments, R⁵ is

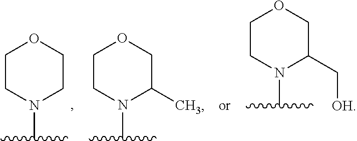

In some embodiments, R⁵ is

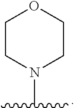 [SMILES: *N1CCOCC1]

In some embodiments, the compound has the structure of Formula Ia:

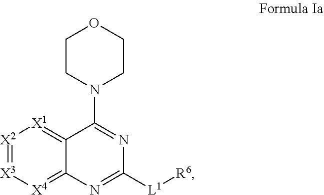

or a pharmaceutically acceptable salt thereof.

In some embodiments, X¹ is N, X² is CR², X³ is CR³, and X⁴ is CR⁴. In some embodiments, X² is N, X¹ is CR¹, X³ is CR³, and X⁴ is CR⁴. In some embodiments, X³ is N, X¹ is CR¹, X² is CR², and X⁴ is CR⁴. In some embodiments, X⁴ is N, X¹ is CR¹, X² is CR², and X³ is CR³.

In some embodiments, the compound has the structure of Formula II:

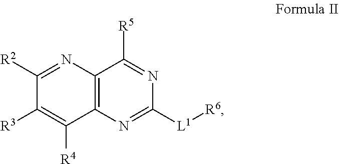

or a pharmaceutically acceptable salt thereof.

In some embodiments, R² is halogen or optionally substituted C₁₋₆ alkyl.

In some embodiments, the compound has the structure of Formula IIa:

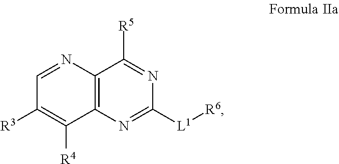

or a pharmaceutically acceptable salt thereof.

In some embodiments, R⁴ is halogen or optionally substituted C₁₋₆ alkyl.

In some embodiments, the compound has the structure of Formula IIb:

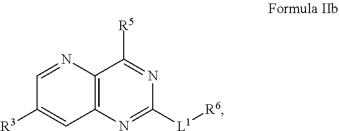

or a pharmaceutically acceptable salt thereof.

In some embodiments, the compound has the structure of Formula IIc:

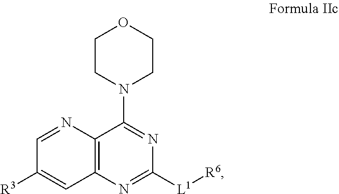

or a pharmaceutically acceptable salt thereof.

In some embodiments, R³ is halogen or optionally substituted C₁₋₆ alkyl.

In some embodiments, R³ is Br.

In some embodiments, R³ is

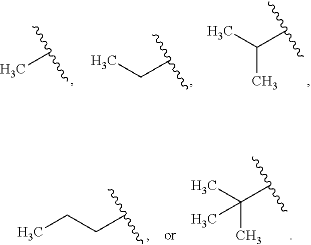

In some embodiments, R³ is

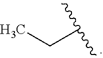 [SMILES: *CC]

In some embodiments, R³ is

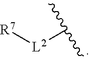

In some embodiments, L² is absent.

In some embodiments, L² is

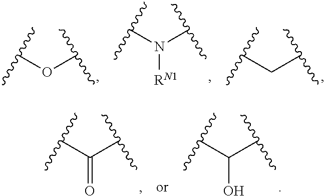

In some embodiments, R^{N1} is hydrogen or

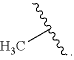

In some embodiments, R^{N1} is hydrogen.

In some embodiments, L² is

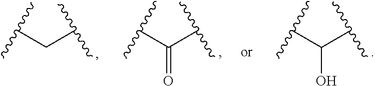

In some embodiments, R⁷ is optionally substituted C₆₋₁₀ aryl.

In some embodiments, R⁷ is optionally substituted C₃₋₁₀ carbocyclyl, optionally substituted C₁₋₉ heteroaryl, or optionally substituted C₁₋₉ heterocyclyl.

In some embodiments, R⁷ is optionally substituted C₃₋₁₀ carbocyclyl.

In some embodiments, R⁷ is

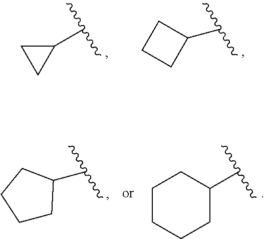

In some embodiments, R⁷ is

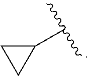 [SMILES: *C1CC1]

In some embodiments, R⁷ is optionally substituted C₁₋₉ heteroaryl or optionally substituted C₁₋₉ heterocyclyl.

In some embodiments, R⁷ is optionally substituted C₁₋₉ heteroaryl.

In some embodiments, R⁷ is

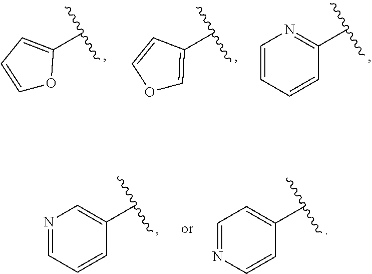 [SMILES: *c1ccco1 ; *c1ccoc1 ; *c1ccccn1 ; *c1cccnc1 ; *c1ccncc1]

In some embodiments, R⁷ is

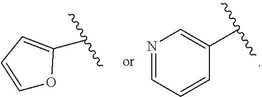 [SMILES: *c1ccco1 ; *c1cccnc1]

In some embodiments, R⁷ is or optionally substituted C₁₋₉ heterocyclyl.

In some embodiments, R⁷ is

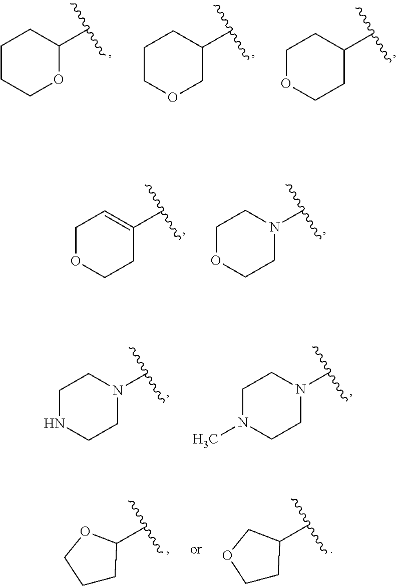

In some embodiments, R⁷ is

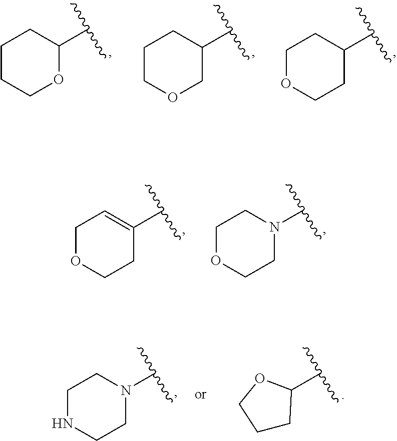

In some embodiments, R⁷ is

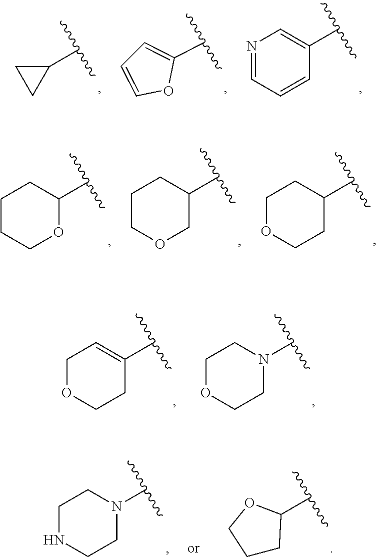

In some embodiments, the compound has the structure of Formula IId:

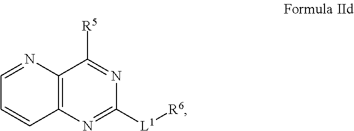

or a pharmaceutically acceptable salt thereof.

In some embodiments, the compound has the structure of Formula IIe:

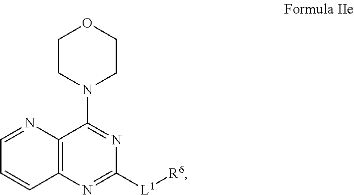

or a pharmaceutically acceptable salt thereof.

In some embodiments, the compound has the structure of Formula IIf:

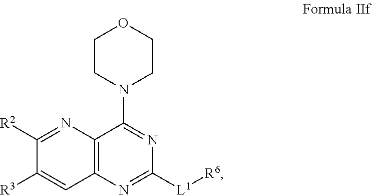

or a pharmaceutically acceptable salt thereof.

In some embodiments, the compound has the structure of Formula IIg:

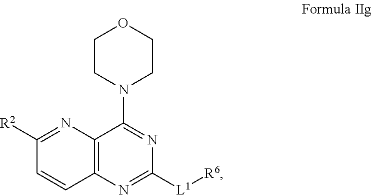

or a pharmaceutically acceptable salt thereof.

In some embodiments, R² is optionally substituted C₁₋₉ heterocyclyl.

In some embodiments, R² is

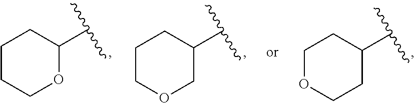 [SMILES: *C1CCCCO1 ; *C1CCCOC1 ; *C1CCOCC1]

In some embodiments, R² is

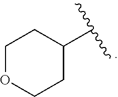 [SMILES: *C1CCOCC1]

In some embodiments, the compound has the structure of Formula III:

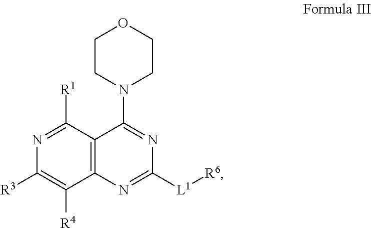

or a pharmaceutically acceptable salt thereof.

In some embodiments, the compound has the structure of Formula IIIa:

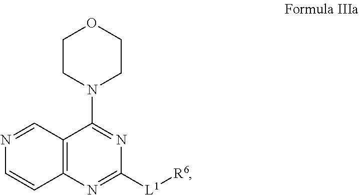

or a pharmaceutically acceptable salt thereof.

In some embodiments, the compound has the structure of Formula IV:

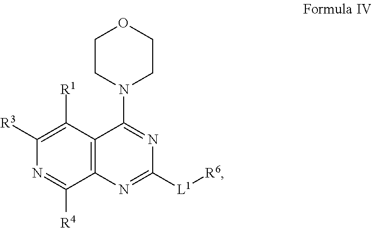

or a pharmaceutically acceptable salt thereof.

In some embodiments, the compound has the structure of Formula IVa:

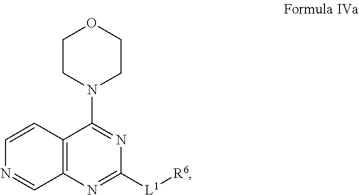

or a pharmaceutically acceptable salt thereof.

In some embodiments, the compound has the structure of Formula V:

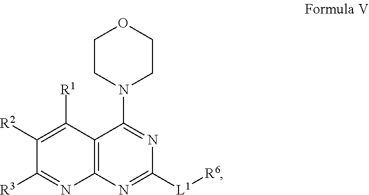

or a pharmaceutically acceptable salt thereof.

In some embodiments, the compound has the structure of Formula Va:

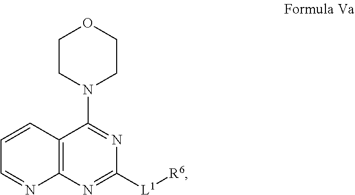

or a pharmaceutically acceptable salt thereof.

In some embodiments, L¹ is absent.

In some embodiments, L¹ is

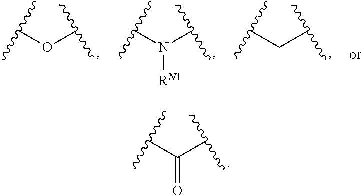

In some embodiments, R^{N1} is hydrogen or

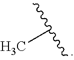

In some embodiments, R^{N1} is hydrogen.

In some embodiments, L¹ is

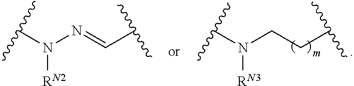

In some embodiments, L¹ is R^{N2}

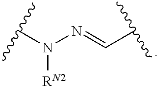

In some embodiments, R^{N2} is hydrogen or

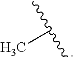

In some embodiments, R^{N2} is hydrogen.

In some embodiments, L¹ is

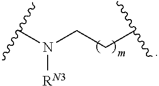

In some embodiments, m is 1.

In some embodiments, R^{N3} is hydrogen or

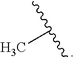

In some embodiments, R^{N3} is hydrogen.

In some embodiments, L¹ is optionally substituted C₁₋₉ heteroarylene or optionally substituted C₁₋₉ heterocyclylene.

In some embodiments, L¹ is optionally substituted C₁₋₉ heteroarylene.

In some embodiments, L¹ is optionally substituted C₁₋₅ heteroarylene.

In some embodiments, L¹ is optionally substituted 6-membered C₁₋₅ heteroarylene.

In some embodiments, L¹ is

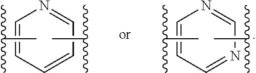

In some embodiments, L¹ is optionally substituted C₁₋₄ heteroarylene.

In some embodiments, L¹ is optionally substituted 5-membered C₁₋₅ heteroarylene.

In some embodiments, L¹ is

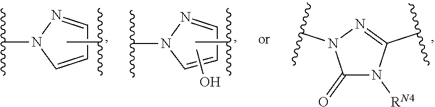

where R^{N4} is hydrogen or optionally substituted C₁₋₆ alkyl.

In some embodiments,

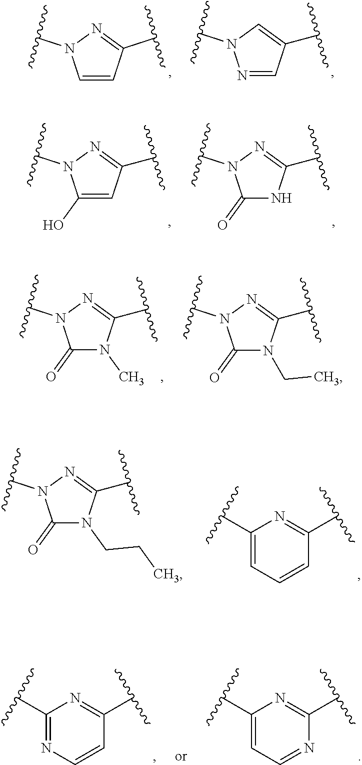

In some embodiments, L¹ is

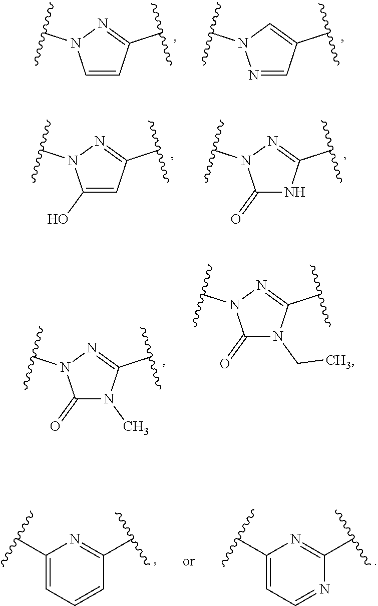

In some embodiments, L¹ is optionally substituted C₁₋₉ heterocyclylene.

In some embodiments, L¹ is optionally substituted C₁₋₅ heterocyclylene.

In some embodiments, L¹ is optionally substituted C₁₋₄ heterocyclylene.

In some embodiments, L¹ is optionally substituted 5-membered C₁₋₅ heterocyclylene.

In some embodiments, L¹ is

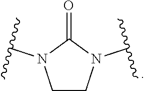 [SMILES: *N1CCN(*)C1=O]

In some embodiments, R⁶ is halogen.

In some embodiments, R⁶ is Cl.

In some embodiments, R⁶ is optionally substituted C₁₋₆ alkyl.

In some embodiments, R⁶ is

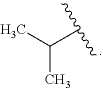 [SMILES: *C(C)C]

In some embodiments, R⁶ is optionally substituted C₆₋₁₀ aryl, optionally substituted C₃₋₁₀ carbocyclyl, optionally substituted C₁₋₉ heteroaryl, optionally substituted C₁₋₉ heterocyclyl, or optionally substituted —C₁₋₆ alkylene-C₁₋₉ heterocyclyl.

In some embodiments, R⁶ is optionally substituted C₆₋₁₀ aryl.

In some embodiments, R⁶ is

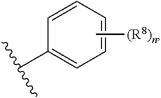

where n is 0, 1, 2, 3, 4, or 5; and each R⁸ is, independently, halogen or optionally substituted C₁₋₆ alkyl.

In some embodiments, each R⁸ is, independently, F or

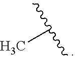

In some embodiments, n is 0 or 1.

In some embodiments, R⁶ is

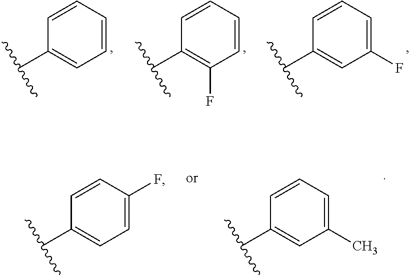

In some embodiments, R⁶ is optionally substituted C₃₋₁₀ carbocyclyl.

In some embodiments, R⁶ is

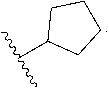 [SMILES: *C1CCCC1]

In some embodiments, R⁶ is optionally substituted C₁₋₉ heteroaryl.

In some embodiments, R⁶ is optionally substituted monocyclic C₁₋₉ heteroaryl.

In some embodiments, R⁶ is

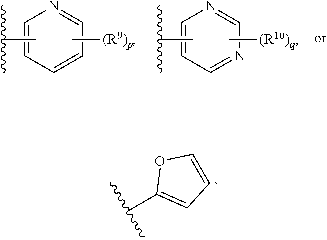

where p is 0, 1, 2, or 3; q is 0, 1, or 2; each R⁹ is, independently, halogen or optionally substituted C₁₋₆ alkyl; and each R¹⁰ is, independently, halogen or optionally substituted C₁₋₆ alkyl.

In some embodiments, p is 0.

In some embodiments, q is 0.

In some embodiments, R⁶ is

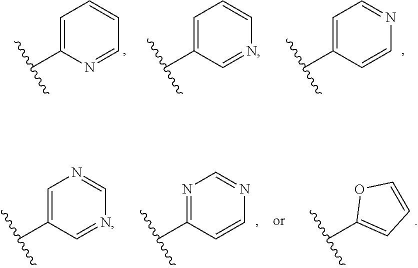 [SMILES: *c1ccccn1 ; *c1cccnc1 ; *c1ccncc1 ; *c1cncnc1 ; *c1ccncn1 ; *c1ccco1]

In some embodiments, R⁶ is optionally substituted polycyclic C₁₋₉ heteroaryl.

In some embodiments, R⁶ is

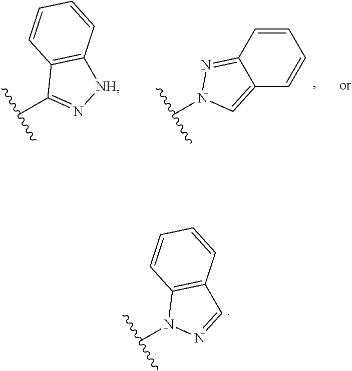 [SMILES: *c1n[nH]c2ccccc12 ; *n1cc2ccccc2n1 ; "," ; "or" ; *n1ncc2ccccc21]

In some embodiments, R⁶ is optionally substituted C₁₋₉ heterocyclyl.

In some embodiments, R⁶ is optionally substituted monocyclic C₁₋₉ heterocyclyl.

In some embodiments, R⁶ is

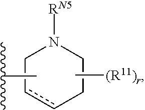

where [symbol] represents a single bond or a double bond; r is 0, 1, 2, 3, 4, 5, or 6; each R¹¹ is, independently, halogen or optionally substituted C₁₋₆ alkyl; and R^{N5} is hydrogen, optionally substituted C₁₋₆ alkyl, or optionally substituted C₁₋₆ heteroalkyl.

In some embodiments, r is 0, 1, or 2.

In some embodiments, R^{N5} is

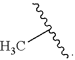

In some embodiments, R⁶ is

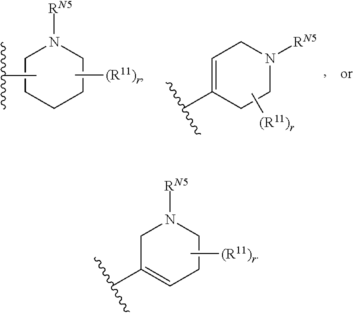

In some embodiments, R⁶ is

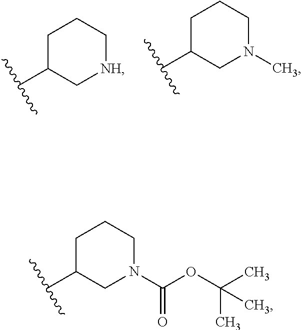 [SMILES: *C1CCCNC1 ; *C1CCCN(C)C1 ; *C1CCCN(C(=O)OC(C)(C)C)C1]

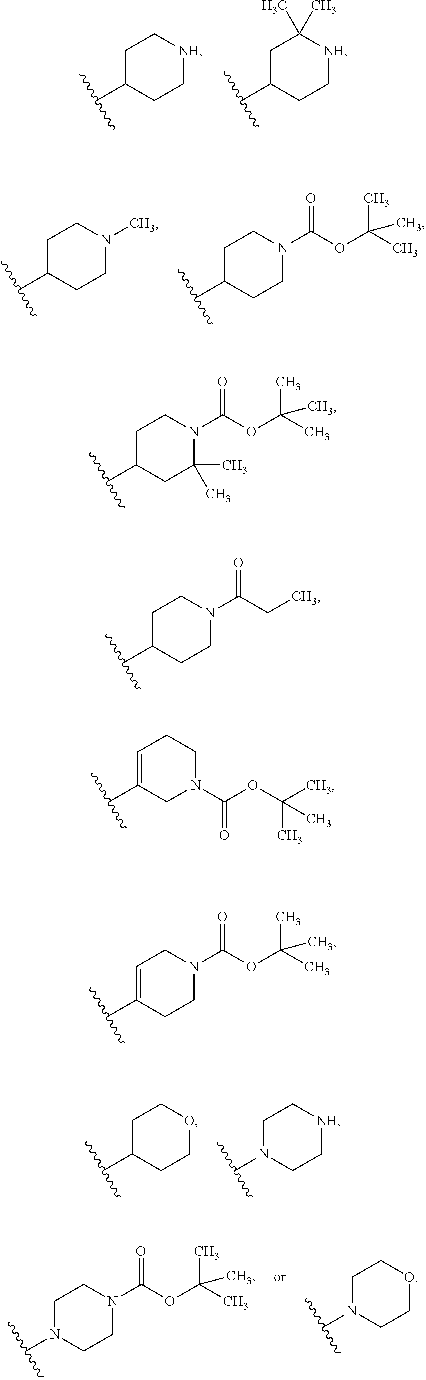

In some embodiments, R⁶ is optionally substituted —C₁₋₆ alkylene-C₁₋₉ heterocyclyl.

In some embodiments, R⁶ is

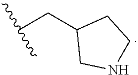 [SMILES: *CC1CCNC1]

In some embodiments, the compound has the structure of any one of compounds 1-88 in Table 1.

In another aspect, the invention features a method of inhibiting PIKfyve in a cell expressing PIKfyve protein, the method including contacting the cell with any of the foregoing compounds, or a pharmaceutically acceptable salt thereof.

In another aspect, the invention features a method of treating a neurological disorder in a patient, such as a human patient, identified as likely to benefit from treatment with a compound of the invention on the basis of TDP-43 toxicity. In this aspect, the method may include (i) determining that the patient exhibits, or is prone to develop, TDP-43 toxicity, and (ii) providing to the patient a therapeutically effective amount of a compound of the invention. In some embodiments, the patient has previously been determined to exhibit, or to be prone to developing, TDP-43 toxicity, and the method includes providing to the patient a therapeutically effective amount of a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In an additional aspect, the invention features a method of treating a neurological disorder in a patient, such as a human patient, identified as likely to benefit from treatment with a compound of the invention on the basis of TDP-43 expression. In this aspect, the method includes (i) determining that the patient expresses a mutant form of TDP-43 having a mutation associated with TDP-43 aggregation (e.g., a mutation selected from Q331K, M337V, Q343R, N345K, R361S, and N390D), and (ii) providing to the patient a therapeutically effective amount of a compound of the invention. In some embodiments, the patient has previously been determined to express a mutant form of TDP-43 having a mutation associated with TDP-43 aggregation, such as a Q331K, M337V, Q343R, N345K, R361S, or N390D mutation, and the method includes providing to the patient a therapeutically effective amount of a compound of the invention.

In another aspect, the invention features a method of determining whether a patient (e.g., a human patient) having a neurological disorder is likely to benefit from treatment with a compound of the invention by (i) determining whether the patient exhibits, or is prone to develop, TDP-43 aggregation and (ii) identifying the patient as likely to benefit from treatment with a compound of the invention if the patient exhibits, or is prone to develop, TDP-43 aggregation. In some embodiments, the method further includes the step of (iii) informing the patient whether he or she is likely to benefit from treatment with a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In another aspect, the invention features a method of determining whether a patient (e.g., a human patient) having a neurological disorder is likely to benefit from treatment with a compound of the invention by (i) determining whether the patient expresses a TDP-43 mutant having a mutation associated with TDP-43 aggregation (e.g., a mutation selected from Q331K, M337V, Q343R, N345K, R361S, and N390D) and (ii) identifying the patient as likely to benefit from treatment with a compound of the invention if the patient expresses a TDP-43 mutant. In some embodiments, the method further includes the step of (iii) informing the patient whether he or she is likely to benefit from treatment with a compound of the invention. The TDP-43 isoform expressed by the patient may be assessed, for example, by isolated TDP-43 protein from a sample obtained from the patient and sequencing the protein using molecular biology techniques described herein or known in the art. In some embodiments, the TDP-43 isoform expressed by the patient is determined by analyzing the patient's genotype at the TDP-43 locus, for example, by sequencing the TDP-43 gene in a sample obtained from the patient. In some embodiments, the method includes the step of obtaining the sample from the patient.

In some embodiments of any of the above aspects, the compound of the invention is provided to the patient by administration of the compound of the invention to the patient. In some embodiments, the compound of the invention is provided to the patient by administration of a prodrug that is converted in vivo to the compound of the invention.

In some embodiments of any of the above aspects, the neurological disorder is a neuromuscular disorder, such as a neuromuscular disorder selected from amyotrophic lateral sclerosis, congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome. In some embodiments, the neurological disorder is amyotrophic lateral sclerosis.

In some embodiments of any of the above aspects, the neurological disorder is selected from frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy.

In some embodiments, the neurological disorder is amyotrophic lateral sclerosis, and following administration of the compound of the invention to the patient, the patient exhibits one or more, or all, of the following responses:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the compound of the invention (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(ii) an increase in slow vital capacity, such as an increase in the patient's slow vital capacity within one or more days, weeks, or months following administration of the compound of the invention (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iii) a reduction in decremental responses exhibited by the patient upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(vi) a decrease in the frequency and/or severity of muscle cramps, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient); and/or
(vii) a decrease in TDP-43 aggregation, such as a decrease in TDP-43 aggregation within one or more days, weeks, or months following administration of the compound of the invention (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient.

### Chemical Terms

It is to be understood that the terminology employed herein is for the purpose of describing particular embodiments and is not intended to be limiting.

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion, e.g., the interconversion illustrated in the scheme below:

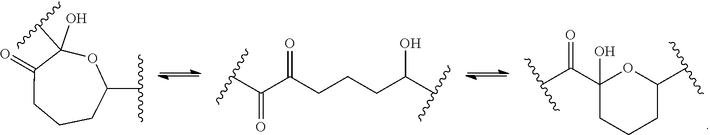 [SMILES: *C1CCCC(=O)C(*)(O)O1 ; *C(=O)C(=O)CCCC(*)O ; *C(=O)C1(O)CCCC(*)O1]

Those skilled in the art will appreciate that, in some embodiments, isotopes of compounds described herein may be prepared and/or utilized in accordance with the present invention. “Isotopes” refers to atoms having the same atomic number but different mass numbers resulting from a different number of neutrons in the nuclei. For example, isotopes of hydrogen include tritium and deuterium. In some embodiments, an isotopic substitution (e.g., substitution of hydrogen with deuterium) may alter the physiciochemical properties of the molecules, such as metabolism and/or the rate of racemization of a chiral center.

As is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized in any form, including in any solid form. In some embodiments, such entities are utilized in a particular form, for example in a particular solid form.

In some embodiments, compounds described and/or depicted herein may be provided and/or utilized in salt form.

In certain embodiments, compounds described and/or depicted herein may be provided and/or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional.

The term “acyl,” as used herein, represents a hydrogen or an alkyl group, as defined herein that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂RN², SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆₋₁₀ aryl, C₁-C₁₀ alkyl C₆₋₁₀ aryl, or C₁-C₂₀ alkyl C₆₋₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “cyano,” as used herein, represents a CN group.

The terms “carbocyclyl,” as used herein, refer to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring containing one, two, three, or four ring heteroatoms selected from N, O, and S, with the remaining ring atoms being C. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, denotes a mono- or polycyclic radical having 3 to 12 atoms having at least one ring containing one, two, three, or four ring heteroatoms selected from N, O or S and no aromatic ring containing any N, O, or S atoms. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl. A heterocyclyl group may be aromatic or non-aromatic. An aromatic heterocyclyl is also referred to as heteroaryl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999). N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an NO₂ group.

The term “oxyheteroaryl,” as used herein, represents a heteroaryl group having at least one endocyclic oxygen atom.

The term “oxyheterocyclyl,” as used herein, represents a heterocyclyl group having at least one endocyclic oxygen atom.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), halo (e.g., fluoro), hydroxyl, oxo, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer.

Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

As used herein, the term “administration” refers to the administration of a composition (e.g., a compound, a complex or a preparation that includes a compound or complex as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

As used herein, the terms “approximately” and “about” are each intended to encompass normal statistical variation as would be understood by those of ordinary skill in the art as appropriate to the relevant context. In certain embodiments, the terms “approximately” or “about” each refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population).

As used herein, the terms “benefit” and “response” are used interchangeably in the context of a subject, such as a human subject undergoing therapy for the treatment of a neurological disorder, for example, amyotrophic lateral sclerosis, frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy. The terms “benefit” and “response” refer to any clinical improvement in the subject's condition. Exemplary benefits in the context of a subject undergoing treatment for a neurological disorder using the compositions and methods described herein (e.g., in the context of a human subject undergoing treatment for a neurological disorder described herein, such as amyotrophic lateral sclerosis, with a FYVE-type zinc finger containing phosphoinositide kinase (PIKfyve) inhibitor described herein, such as an inhibitory small molecule, antibody, antigen-binding fragment thereof, or interfering RNA molecule) include the slowing and halting of disease progression, as well as suppression of one or more symptoms associated with the disease. Particularly, in the context of a patient (e.g., a human patient) undergoing treatment for amyotrophic lateral sclerosis with a compound of the invention, examples of clinical “benefits” and “responses” are (i) an improvement in the subject's condition as assessed using the amyotrophic lateral sclerosis functional rating scale (ALSFRS) or the revised ALSFRS (ALSFRS-R) following administration of the compound of the invention, such as an improvement in the subject's ALSFRS or ALSFRS-R score within one or more days, weeks, or months following administration of the compound of the invention (e.g., an improvement in the subject's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (ii) an increase in the subject's slow vital capacity following administration of the compound of the invention, such as an increase in the subject's slow vital capacity within one or more days, weeks, or months following administration of the compound of the invention (e.g., an increase in the subject's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iii) a reduction in decremental responses exhibited by the subject upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iv) an improvement in the subject's muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (v) an improvement in the subject's quality of life, as assessed, for example, using the amyotrophic lateral sclerosis-specific quality of life (ALS-specific QOL) questionnaire, such as an improvement in the subject's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); and (vi) a decrease in the frequency and/or severity of muscle cramps exhibited by the subject, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject).

As used herein, the term “dosage form” refers to a physically discrete unit of an active compound (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

In the practice of the methods of the present invention, an “effective amount” of any one of the compounds of the invention or a combination of any of the compounds of the invention or a pharmaceutically acceptable salt thereof, is administered via any of the usual and acceptable methods known in the art, either singly or in combination.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of formula (I). For example pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

The terms “PIKfyve” and “FYVE-type zinc finger containing phosphoinositide kinase” are used interchangeably herein and refer to the enzyme that catalyzes phosphorylation of phosphatidylinositol 3-phosphate to produce phosphatidylinositol 3,5-bisphosphate, for example, in human subjects. The terms “PIKfyve” and “FYVE-type zinc finger containing phosphoinositide kinase” refer not only to wild-type forms of PIKfyve, but also to variants of wild-type PIKfyve proteins and nucleic acids encoding the same. The gene encoding PIKfyve can be accessed under NCBI Reference Sequence No. NG_021188.1. Exemplary transcript sequences of wild-type form of human PIKfyve can be accessed under NCBI Reference Sequence Nos. NM_015040.4, NM_152671.3, and NM_001178000.1. Exemplary protein sequences of wild-type form of human PIKfyve can be accessed under NCBI Reference Sequence Nos. NP_055855.2, NP_689884.1, and NP_001171471.1.

As used herein, the term “PIKfyve inhibitor” refers to substances, such as compounds of Formula I. Inhibitors of this type may, for example, competitively inhibit PIKfyve activity by specifically binding the PIKfyve enzyme (e.g., by virtue of the affinity of the inhibitor for the PIKfyve active site), thereby precluding, hindering, or halting the entry of one or more endogenous substrates of PIKfyve into the enzyme's active site. Additional examples of PIKfyve inhibitors that suppress the activity of the PIKfyve enzyme include substances that may bind PIKfyve at a site distal from the active site and attenuate the binding of endogenous substrates to the PIKfyve active site by way of a change in the enzyme's spatial conformation upon binding of the inhibitor. In addition to encompassing substances that modulate PIKfyve activity, the term “PIKfyve inhibitor” refers to substances that reduce the concentration and/or stability of PIKfyve mRNA transcripts in vivo, as well as those that suppress the translation of functional PIKfyve enzyme.

The term “pure” means substantially pure or free of unwanted components (e.g., other compounds and/or other components of a cell lysate), material defilement, admixture or imperfection.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

A variety of clinical indicators can be used to identify a patient as “at risk” of developing a particular neurological disease. Examples of patients (e.g., human patients) that are “at risk” of developing a neurological disease, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include (i) subjects exhibiting or prone to exhibit aggregation of TAR-DNA binding protein (TDP)-43, and (ii) subjects expressing a mutant form of TDP-43 containing a mutation associated with TDP-43 aggregation and toxicity, such as a mutation selected from Q331K, M337V, Q343R, N345K, R361S, and N390D. Subjects that are “at risk” of developing amyotrophic lateral sclerosis may exhibit one or both of these characteristics, for example, prior to the first administration of a PIKfyve inhibitor in accordance with the compositions and methods described herein.

As used herein, the terms “TAR-DNA binding protein-43” and “TDP-43” are used interchangeably and refer to the transcription repressor protein involved in modulating HIV-1 transcription and alternative splicing of the cystic fibrosis transmembrane conductance regulator (CFTR) pre-mRNA transcript, for example, in human subjects. The terms “TAR-DNA binding protein-43” and “TDP-43” refer not only to wild-type forms of TDP-43, but also to variants of wild-type TDP-43 proteins and nucleic acids encoding the same. The amino acid sequence and corresponding mRNA sequence of a wild-type form of human TDP-43 are provided under NCBI Reference Sequence Nos. NM_007375.3 and NP_031401.1, respectively.

The terms “TAR-DNA binding protein-43” and “TDP-43” as used herein include, for example, forms of the human TDP-43 protein that have an amino acid sequence that is at least 85% identical to the amino acid sequence of NCBI Reference Sequence No. NP_031401.1 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NP_031401.1) and/or forms of the human TDP-43 protein that contain one or more substitutions, insertions, and/or deletions (e.g., one or more conservative and/or nonconservative amino acid substitutions, such as up to 5, 10, 15, 20, 25, or more, conservative or nonconservative amino acid substitutions) relative to a wild-type TDP-43 protein. For instance, patients that may be treated for a neurological disorder as described herein, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include human patients that express a form of TDP-43 having a mutation associated with elevated TDP-43 aggregation and toxicity, such as a mutation selected from Q331K, M337V, Q343R, N345K, R361S, and N390D. Similarly, the terms “TAR-DNA binding protein-43” and “TDP-43” as used herein include, for example, forms of the human TDP-43 gene that encode an mRNA transcript having a nucleic acid sequence that is at least 85% identical to the nucleic acid sequence of NCBI Reference Sequence No. NM_007375.3 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NM_007375.3).

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

A “therapeutic regimen” refers to a dosing regimen whose administration across a relevant population is correlated with a desired or beneficial therapeutic outcome.

The term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### BRIEF DESCRIPTION OF THE DRAWINGS

FIG. 1 is a scheme showing an approach to generation of a control TDP-43 yeast model (FAB1 TDP-43). A control yeast TDP-43 model was generated by integrating the human TDP-43 gene and the GAL1 promoter into the yeast genome. The yeast ortholog of human PIKFYVE is FAB1.

FIG. 2 is a scheme showing an approach to generation of a humanized PIKFYVE TDP-43 yeast model (PIKFYVE TDP-43). FAB1 gene through homologous recombination with a G418 resistance cassette (fab1::G418^{R}) (FIG. 2). PIKFYVE was cloned downstream of the GPD promoter harbored on a URA3-containing plasmid and introduced into the fab1::G418R ura3 strain. The pGAL1-TDP-43 construct was then introduced into the “humanized” yeast strain and assessed for cytotoxicity.

FIG. 3 is a histogram generated from the flow cytometry-based viability assay of FAB1 TDP-43.

FIG. 4 is a histogram generated from the flow cytometry-based viability assay of PIKFYVE TDP-43. Upon induction of TDP-43, there was a marked increase in inviable cells (rightmost population), with a more pronounced effect in PIKFYVE TDP-43 than in FAB1 TDP-43 strain (see FIG. 3).

FIG. 5 is an overlay of histograms generated from the flow cytometry-based viability assay of FAB1 TDP-43 in the presence of APY0201.

FIG. 6 is an overlay of histograms generated from the flow cytometry-based viability assay of PIKFYVE TDP-43 in the presence of APY0201.

FIG. 7 is a scatter plot comparing cytoprotection efficacy in PIKFYVE TDP-43 to PIKfyve inhibitory activity of test compounds.

### DETAILED DESCRIPTION

The present invention features compositions and methods for treating neurological disorders, such as amyotrophic lateral sclerosis and other neuromuscular disorders, as well as frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy among others. Particularly, the invention provides inhibitors of FYVE-type zinc finger containing phosphoinositide kinase (PIKfyve), that may be administered to a patient (e.g., a human patient) so as to treat or prevent a neurological disorder, such as one or more of the foregoing conditions. In the context of therapeutic treatment, the PIKfyve inhibitor may be administered to the patient to alleviate one or more symptoms of the disorder and/or to remedy an underlying molecular pathology associated with the disease, such as to suppress or prevent aggregation of TAR-DNA binding protein (TDP)-43.

The disclosure herein is based, in part, on the discovery that PIKfyve inhibition modulates TDP-43 aggregation in cells. Suppression of TDP-43 aggregation exerts beneficial effects in patients suffering from a neurological disorder. Many pathological conditions have been correlated with TDP-43-promoted aggregation and toxicity, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy. Without being limited by mechanism, by administering an inhibitor of PIKfyve, patients suffering from diseases associated with TDP-43 aggregation and toxicity may be treated, for example, due to the suppression of TDP-43 aggregation induced by the PIKfyve inhibitor.

Patients that are likely to respond to PIKfyve inhibition as described herein include those that have or are at risk of developing TDP-43 aggregation, such as those that express a mutant form of TDP-43 associated with TDP-43 aggregation and toxicity in vivo. Examples of such mutations in TDP-43 that have been correlated with elevated TDP-43 aggregation and toxicity include Q331K, M337V, Q343R, N345K, R361S, and N390D, among others. The compositions and methods described herein thus provide the additional clinical benefit of enabling the identification of patients that are likely to respond to PIKfyve inhibitor therapy, as well as processes for treating these patients accordingly.

The sections that follow provide a description of exemplary PIKfyve inhibitors that may be used in conjunction with the compositions and methods disclosed herein. The sections below additionally provide a description of various exemplary routes of administration and pharmaceutical compositions that may be used for delivery of these substances for the treatment of a neurological disorder.

### PIKfyve Inhibitors

Exemplary PIKfyve inhibitors described herein include compounds of Formula I:

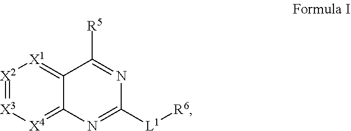

or a pharmaceutically acceptable salt thereof,
where
X¹ is N or CR¹;
X² is N or CR²;
X³ is N or CR³;
X⁴ is N or CR⁴;
R⁵ is

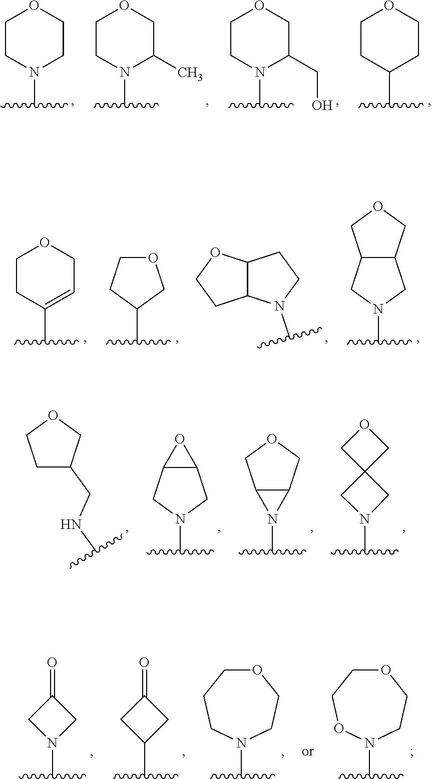

L¹ is absent,

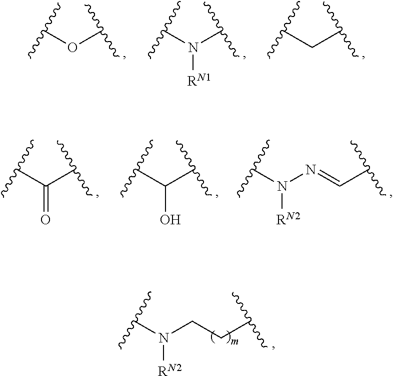

optionally substituted C₁₋₉ heteroarylene, or optionally substituted C₁₋₉ heterocyclylene;
R⁶ is halogen, optionally substituted C₁₋₆ alkyl, optionally substituted C₆₋₁₀ aryl, optionally substituted C₃₋₁₀ carbocyclyl, optionally substituted C₁₋₉ heteroaryl, optionally substituted C₁₋₉ heterocyclyl, or optionally substituted —C₁₋₆ alkylene-C₁₋₉ heterocyclyl,
R¹ is hydrogen, halogen, optionally substituted C₁₋₆ alkyl, or

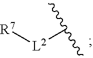

R² is hydrogen, halogen, optionally substituted C₁₋₆ alkyl, or

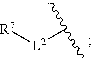

R³ is hydrogen, halogen, optionally substituted C₁₋₆ alkyl, or

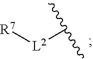

R⁴ is hydrogen, halogen, optionally substituted C₁₋₆ alkyl, or

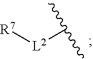

L² is absent,

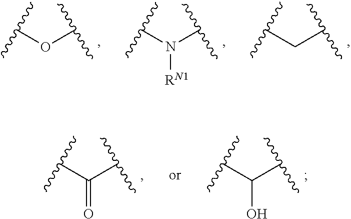

R⁷ is optionally substituted C₆₋₁₀ aryl, optionally substituted C₃₋₁₀ carbocyclyl, optionally substituted C₁₋₉ heteroaryl, or optionally substituted C₁₋₉ heterocyclyl;
each of R^{N1}, R^{N2} and R^{N3} is, independently, hydrogen or optionally substituted C₁₋₆ alkyl; and
m is 0, 1, 2, or 3;
wherein one and only one of X¹, X², X³, and X⁴ is N.

In some embodiments, L¹ is

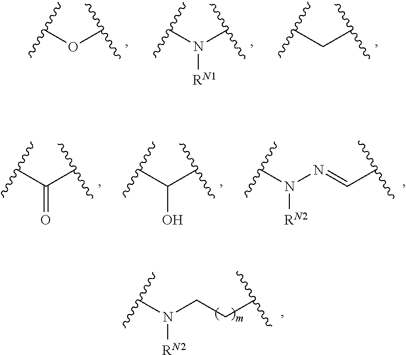

optionally substituted C₁₋₉ heteroarylene having at least one 5-membered ring, or optionally substituted non-aromatic C₁₋₉ heterocyclylene; and R⁶ is optionally substituted C₁₋₆ alkyl, optionally substituted C₆₋₁₀ aryl, optionally substituted C₃₋₁₀ carbocyclyl, optionally substituted C₁₋₉ heteroaryl, optionally substituted C₁₋₉ heterocyclyl, or optionally substituted —C₁₋₆ alkylene-C₁₋₉ heterocyclyl; or L¹ and R⁶ combine to form an optionally substituted C₂₋₉ oxyheteroaryl, optionally substituted pyrimidin-4-yl, or optionally substituted pyrid-2-yl.

Preferably, the compound is of formula IIb:

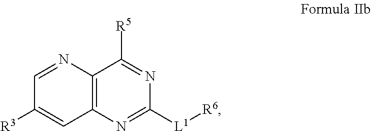

or a pharmaceutically acceptable salt thereof, where the variables are as described herein.

### Methods of Treatment

Suppression of PIKfyve Activity and TDP-43 Aggregation to Treat Neurological Disorders Using the compositions and methods described herein, a patient suffering from a neurological disorder may be administered a PIKfyve inhibitor, such as a small molecule described herein, so as to treat the disorder and/or to suppress one or more symptoms associated with the disorder. Exemplary neurological disorders that may be treated using the compositions and methods described herein are, without limitation, amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy, as well as neuromuscular diseases such as congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome.

The present disclosure is based, in part, on the discovery that PIKfyve inhibitors, such as the agents described herein, are capable of attenuating TDP-43 toxicity. TDP-43-promoted toxicity has been associated with various neurological diseases. The discovery that PIKfyve inhibitors modulate TDP-43 aggregation provides an important therapeutic benefit. Using a PIKfyve inhibitor, such as a PIKfyve inhibitor described herein, a patient suffering from a neurological disorder or at risk of developing such a condition may be treated in a manner that remedies an underlying molecular etiology of the disease. Without being limited by mechanism, the compositions and methods described herein can be used to treat or prevent such neurological conditions, for example, by suppressing the TDP-43 aggregation that promotes pathology.

Additionally, the compositions and methods described herein provide the beneficial feature of enabling the identification and treatment of patients that are likely to respond to PIKfyve inhibitor therapy. For example, in some embodiments, a patient (e.g., a human patient suffering from or at risk of developing a neurological disease described herein, such as amyotrophic lateral sclerosis) is administered a PIKfyve inhibitor if the patient is identified as likely to respond to this form of treatment. Patients may be identified as such on the basis, for example, of susceptibility to TDP-43 aggregation. In some embodiments, the patient is identified is likely to respond to PIKfyve inhibitor treatment based on the isoform of TDP-43 expressed by the patient. For example, patients expressing TDP-43 isoforms having a mutation selected from Q331K, M337V, Q343R, N345K, R361S, and N390D, among others, are more likely to develop TDP-43-promoted aggregation and toxicity relative to patients that do not express such isoforms of TDP-43. Using the compositions and methods described herein, a patient may be identified as likely to respond to PIKfyve inhibitor therapy on the basis of expressing such an isoform of TDP-43, and may subsequently be administered a PIKfyve inhibitor so as to treat or prevent one or more neurological disorders, such as one or more of the neurological disorders described herein.

### Assessing Patient Response

A variety of methods known in the art and described herein can be used to determine whether a patient having a neurological disorder (e.g., a patient at risk of developing TDP-43 aggregation, such as a patient expressing a mutant form of TDP-43 having a mutation associated with elevated TDP-43 aggregation and toxicity, for example, a mutation selected from Q331K, M337V, Q343R, N345K, R361S, and N390D) is responding favorably to PIKfyve inhibition. For example, successful treatment of a patient having a neurological disease, such as amyotrophic lateral sclerosis, with a PIKfyve inhibitor described herein may be signaled by:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the PIKfyve inhibitor (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(ii) an increase in slow vital capacity, such as an increase in the patient's slow vital capacity within one or more days, weeks, or months following administration of the PIKfyve inhibitor (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iii) a reduction in decremental responses exhibited by the patient upon repetitive nerve stimulation, such as a reduction that is observed within one or more days, weeks, or months following administration of the PIKfyve inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the PIKfyve inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the PIKfyve inhibitor (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(vi) a decrease in the frequency and/or severity of muscle cramps, such as a decrease in cramp frequency and/or severity within one or more days, weeks, or months following administration of the PIKfyve inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient); and/or
(vii) a decrease in TDP-43 aggregation, such as a decrease in TDP-43 aggregation within one or more days, weeks, or months following administration of the PIKfyve inhibitor (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient.

### Combination Formulations and Uses Thereof

The compounds of the invention can be combined with one or more therapeutic agents. In particular, the therapeutic agent can be one that treats or prophylactically treats any neurological disorder described herein.

### Combination Therapies

A compound of the invention can be used alone or in combination with other agents that treat neurological disorders or symptoms associated therewith, or in combination with other types of treatment to treat, prevent, and/or reduce the risk of any neurological disorders. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

### Pharmaceutical Compositions

The compounds of the invention are preferably formulated into pharmaceutical compositions for administration to human subjects in a biologically compatible form suitable for administration in vivo. Accordingly, in another aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers.

A compound of the invention may also be administered parenterally. Solutions of a compound of the invention can be prepared in water suitably mixed with a surfactant. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20^{th} ed.) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg.

Alternatively, the dosage amount can be calculated using the body weight of the patient. For example, the dose of a compound, or pharmaceutical composition thereof, administered to a patient may range from 0.1-50 mg/kg.

The following examples are meant to illustrate the invention. They are not meant to limit the invention in any way.

### EXAMPLES

### List of Abbreviations

NEt₃=triethylamine
THE=tetrahydrofuran
Cs₂CO₃=cesium carbonate
Pd(PPh₃)Cl₂=Bis(triphenylphosphine)palladium(II) dichloride
Pd(dppf)Cl₂=[1,1′-Bis(diphenylphosphino)ferrocene]dichloropalladium(II)
K₂CO₃=potassium carbonate
H₂O=water
Pd/C=palladium on carbon
Pd(PPh₃)₄=palladium tetrakis
Na₂CO₃=sodium carbonate
DMF=N,N-dimethylformamide
DIPEA=N,N-diisopropylethylamine
H₂=hydrogen
MeOH=methanol
HCl=hydrochloric acid
EtOAc=ethyl acetate
DCM=dichloromethane
POCl₃=phosphorus oxychloride
AcOH=acetic acid
EtOH=ethanol
N₂H₄H₂O=hydrazine hydrate
NaH=sodium hydride
DMF-DMA=N,N-dimethylformamide dimethyl acetal
EtONa=sodium ethoxide
KF=potassium fluoride

### Example 1. Preparation of Compounds—General Schemes

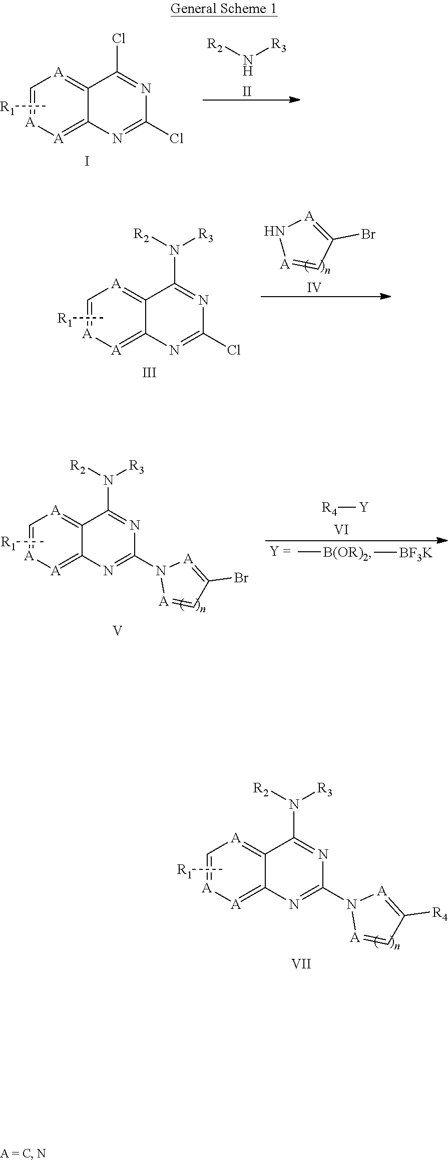

An appropriately substituted dichloropyrimidine I is coupled with appropriately substituted amine II under basic conditions (e.g. triethylamine) to afford appropriately substituted aryl chloride III. Aryl chloride III is reacted with appropriately substituted amine IV under basic conditions (e.g. cesium carbonate) to afford appropriately substituted aryl bromide V. Aryl bromide V is coupled with appropriately substituted boronate ester VI in the presence of a palladium catalyst (e.g. palladium tetrakis) to afford desired product VII.

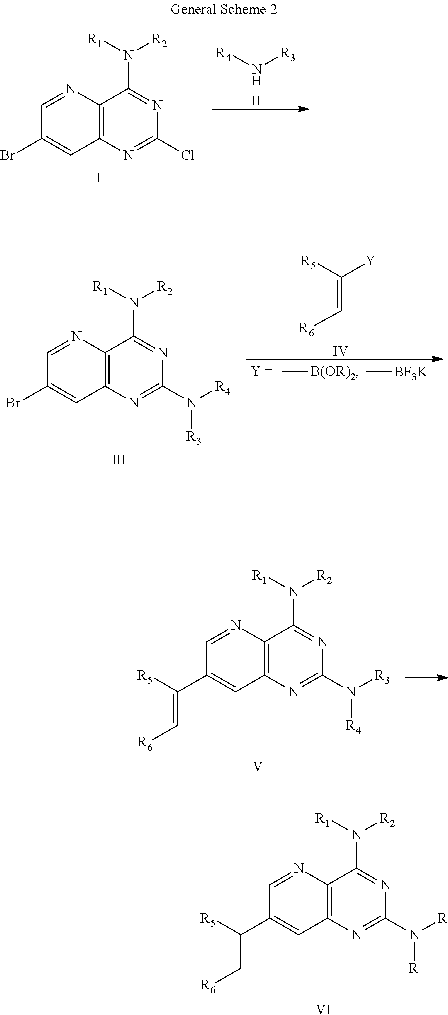

An appropriately substituted aryl chloride I is reacted with amine II under basic conditions (e.g. cesium carbonate) to afford appropriately substituted aryl bromide III. Aryl bromide III can be coupled with appropriately substituted boronate ester IV in the presence of a palladium catalyst (e.g. palladium tetrakis) to afford appropriately substituted pyridopyrimidine V, which can be hydrogenated in the presence of palladium on carbon to afford desired pyridopyrimidine VI.

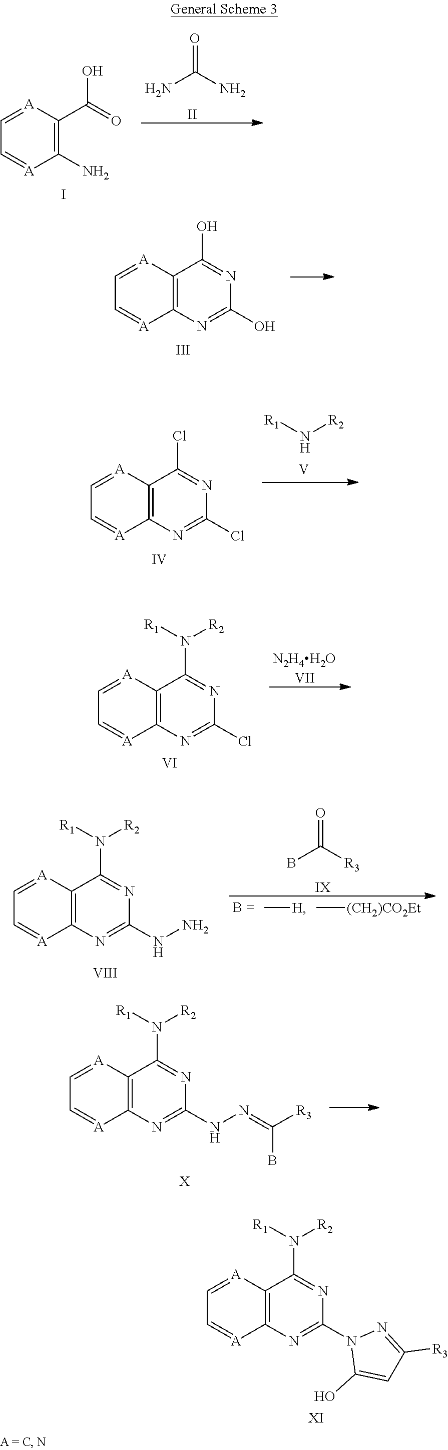

An appropriately substituted carboxylic acid I and urea II are reacted with heat to give appropriately substituted diol III, which is chlorinated with phosphorus oxychloride to give appropriately substituted aryl chloride IV. Aryl chloride IV is reacted with appropriately substituted amine V under basic conditions (e.g. triethylamine) to give appropriately substituted aryl chloride VI. Aryl chloride VI is reacted with hydrazine hydrate VII with heat to give appropriately substituted hydrazine VII. Hydrazine VII is reacted with appropriately substituted aldehyde or enone IX under acidic conditions (e.g. acetic acid) to give desired hydrazone X. When “B” is appropriately substituted enone IX, hydrazone X can be further cyclized to give desired hydroxypyrazole XI.

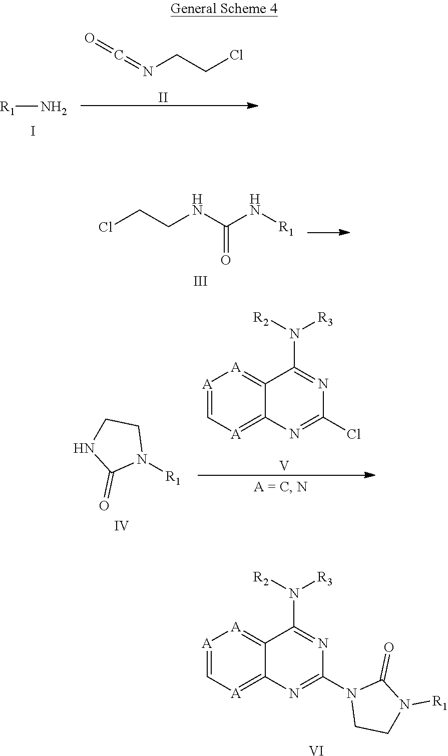

An appropriately substituted amine I is reacted with 1-chloro-2-isocyanatoethane II to give appropriately substituted urea III. Urea III is cyclized under basic conditions (e.g. sodium hydride) to give cyclic urea IV. Urea IV is reacted with appropriately substituted aryl chloride V to give desired product VI.

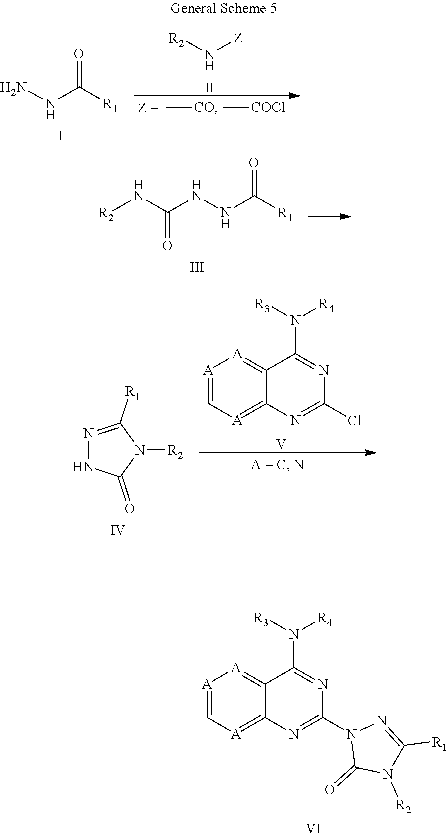

An appropriately substituted hydrazine I is reacted with appropriately substituted isocyanate or carbamoyl chloride II to give appropriately substituted carbohydrazide III, which is cyclized under basic conditions (e.g. sodium hydroxide) to give appropriately substituted triazolone IV. Triazolone IV is reacted with appropriately substituted aryl chloride V under basic conditions (e.g. cesium carbonate) to give desired product VI.

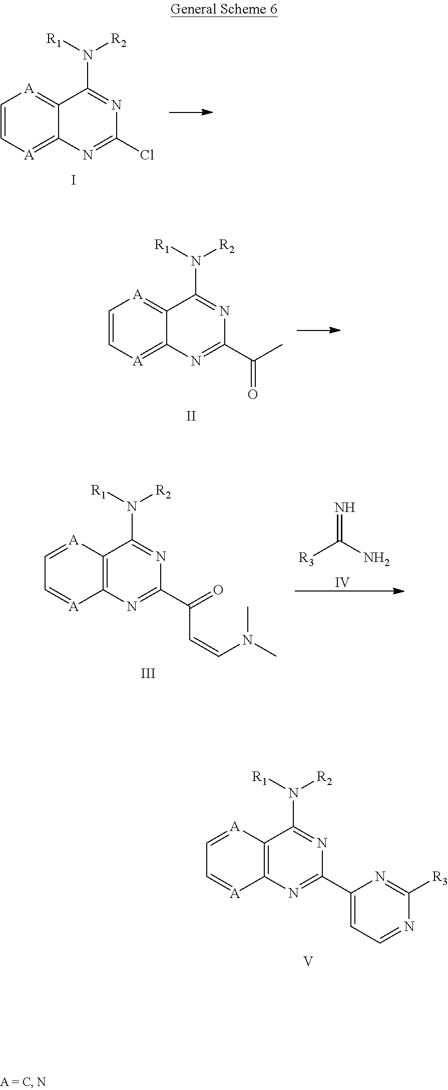

An appropriately substituted aryl chloride I is reacted with tributyl(1-ethoxyvinyl)stannane in the presence of a palladium catalyst (e.g. bis(triphenylphosphine)palladium(II) dichloride) to afford appropriately substituted acetylpyrimidine II. Acetylpyrimidine II is reacted with N,N-dimethylformamide dimethyl acetal to afford appropriately substituted enone III. Enone III is reacted with appropriately substituted amidine IV under basic conditions (e.g. sodium ethoxide) to afford desired pyrimidine V.

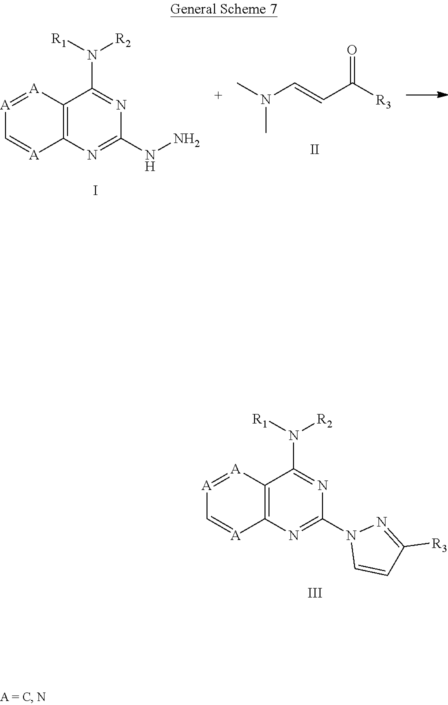

An appropriately substituted hydrazine I is reacted with an appropriately substituted enone II under acidic conditions (e.g. acetic acid) to afford desired pyrazole III.

### Synthesis of tert-butyl 4-[1-(4-morpholinopyrido[3,2-d]pyrimidin-2-yl)pyrazol-3-yl]-3,6-dihydro-2H-pyridine-1-carboxylate (Compound 3), tert-butyl 4-[1-(4-morpholinopyrido[3,2-d]pyrimidin-2-yl)pyrazol-3-yl]piperidine-1-carboxylate (Compound 5), 4-[2-[3-(4-piperidyl)pyrazol-1-yl]pyrido[3,2-d]pyrimidin-4-yl]morpholine (Compound 42) and 1-[4-[1-(4-morpholinopyrido[3,2-d]pyrimidin-2-yl)pyrazol-3-yl]-1-piperidyl]propan-1-one (Compound 1)

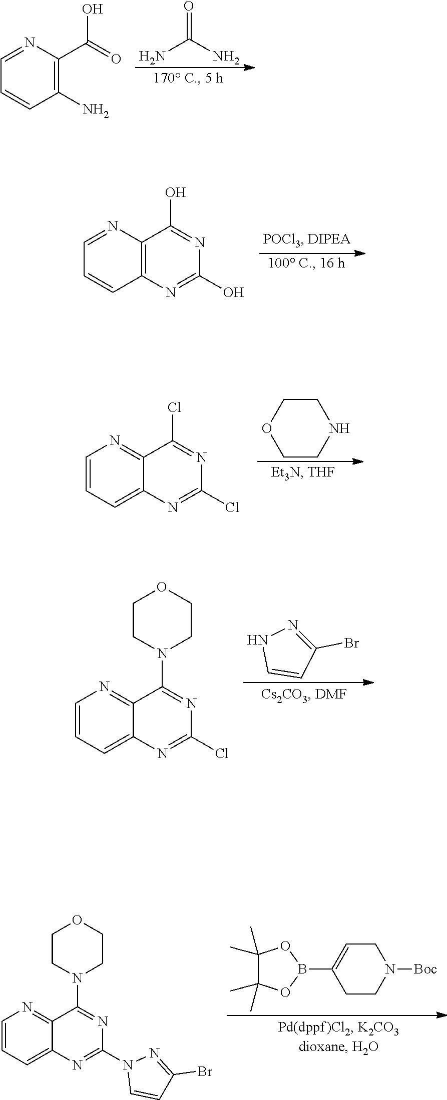

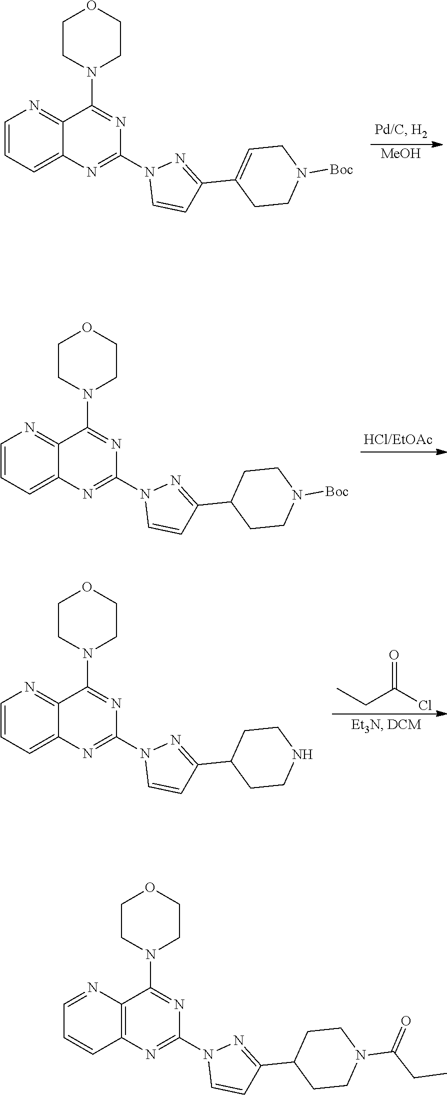

### Step 1: Synthesis of pyrido[3,2-d]pyrimidine-2,4-diol

A solution of 3-aminopicolinic acid (4.00 g, 29 mmol) and urea (2.60 g, 44 mmol) in ethanol (5.0 mL) was stirred at 170° C. under nitrogen atmosphere for 6 h. The reaction mixture was concentrated under reduced pressure and deionized water (100 mL) added to the residue. The resultant solution was then acidified with 1.5 M hydrochloric acid solution until a precipitated was formed. The precipitate was collected by filtration, washed with water (2×50 mL) and methanol (2×50 mL) to obtain pyrido[3,2-d]pyrimidine-2,4-diol (3.00 g, 63%) as white solid. ¹H NMR (400 MHz, Dimethylsulfoxide-d₆) δ 11.30 (bs, 2H), 8.44 (d, J=3.0 Hz, 1H), 7.70-7.48 (m, 2H); LCMS (ESI) m/z: 164.1 [M+H]⁺.

### Step 2: Synthesis of 2,4-dichloropyrido[3,2-d]pyrimidine

To a mixture of pyrido[3,2-d]pyrimidine-2,4-diol (1.60 g, 10 mmol) and phosphorus oxychloride (30 mL) was added N,N-diisopropylethylamine (2 mL) and the reaction mixture was stirred at 130° C. for 10 h. The reaction mixture was then concentrated under reduced pressure, and the volatiles were azeotroped with toluene (2×100 mL). The obtained residue was dissolved in ethyl acetate, filtered over celite and the filtrate was concentrated under reduced pressure to obtain 2,4-dichloropyrido[3,2-d]pyrimidine (1.30 g, 65%) as white solid. LCMS (ESI) m/z: 200.0 [M+H]⁺.

### Step 3: Synthesis of 4-(2-chloropyrido[3,2-d]pyrimidin-4-yl)morpholine

To a solution of 2,4-dichloropyrido[3,2-d]pyrimidine (5 g, 25.00 mmol) in THE (100 mL) was added morpholine (2.29 g, 26.25 mmol) and Et₃N (2.66 g, 26.25 mmol) at 0° C. The mixture was warmed up and stirred at 20° C. for 3 h and concentrated. The residue was dissolved in 150 mL chloroform, washed three times with saturated aqueous sodium bicarbonate solution, dried over Na₂SO₄, filtered and concentrated to obtain 4-(2-chloropyrido[3,2-d]pyrimidin-4-yl)morpholine (5.6 g, 89%) as a pale yellow solid. ¹H NMR (400 MHz, CHLOROFORM-d) δ=8.67 (dd, J=4.2, 1.8 Hz, 1H), 8.01 (dd, J=8.6, 1.8 Hz, 1H), 7.60 (dd, J=8.6, 4.2 Hz, 1H), 4.57 (bs, 4H), 3.86 (t, J=4.8 Hz, 4H).

### Step 4: Synthesis of 4-[2-(3-bromopyrazol-1-yl)pyrido[3,2-d]pyrimidin-4-yl]morpholine

To a solution of 4-(2-chloropyrido[3,2-d]pyrimidin-4-yl)morpholine (2 g, 7.98 mmol) in DMF (40 mL) were added 3-bromo-1H-pyrazole (1.17 g, 7.98 mmol) and Cs₂CO₃ (5.20 g, 15.96 mmol). The mixture was stirred at 100° C. for 16 h. 50 mL of water was added to the reaction mixture and it was extracted with ethyl acetate (60 mL*2). The combined organic layers were washed with brine (30 mL), dried over Na₂SO₄ and concentrated to obtain 4-[2-(3-bromopyrazol-1-yl)pyrido[3,2-d]pyrimidin-4-yl]morpholine (2.5 g, 87%) as a yellow solid. ¹H NMR (400 MHz, CHLOROFORM-d) δ=8.64 (dd, J=3.9, 1.8 Hz, 1H), 8.46 (d, J=2.6 Hz, 1H), 8.23 (dd, J=8.6, 1.5 Hz, 1H), 7.60 (dd, J=8.6, 4.2 Hz, 1H), 6.47 (d, J=2.6 Hz, 1H), 4.60 (bs, 4H), 3.91 (t, J=4.8 Hz, 4H).

### Step 5: Synthesis of tert-butyl 4-[1-(4-morpholinopyrido[3,2-d]pyrimidin-2-yl)pyrazol-3-yl]-3,6-dihydro-2H-pyridine-1-carboxylate

To a solution of 4-[2-(3-bromopyrazol-1-yl)pyrido[3,2-d]pyrimidin-4-yl]morpholine (0.5 g, 1.38 mmol) in dioxane (5 mL) and H₂O (1 mL) were added tert-butyl 4-(4,4,5,5-tetramethyl-1,3,2-dioxaborolan-2-yl)-3,6-dihydro-2H-pyridine-1-carboxylate (642 mg, 2.08 mmol), K₂CO₃ (478 mg, 3.46 mmol) and Pd(dppf)Cl₂ (101 mg, 0.138 mmol). The mixture was stirred at 60° C. for 3 h under nitrogen and then 15 mL of water was added to the mixture. It was then extracted with ethyl acetate (30 mL*2), washed with brine (15 mL) and dried over Na₂SO₄. The combined organic layer was concentrated and the crude product was purified by flash column chromatography (ISCO 10 g silica, 10-30% ethyl acetate in petroleum ether, gradient over 20 min) to obtain tert-butyl 4-[1-(4-morpholinopyrido[3,2-d]pyrimidin-2-yl)pyrazol-3-yl]-3,6-dihydro-2H-pyridine-1-carboxylate (400 mg, 62%) as a pale yellow solid. ¹H NMR (400 MHz, CHLOROFORM-d) 5=8.63 (dd, J=4.0, 1.3 Hz, 1H), 8.54 (d, J=2.6 Hz, 1H), 8.21 (dd, J=8.5, 1.2 Hz, 1H), 7.60 (dd, J=8.4, 4.2 Hz, 1H), 6.55 (d, J=2.4 Hz, 1H), 6.35 (bs, 1H), 4.62 (bs, 4H), 4.16-4.07 (m, 2H), 3.93 (t, J=4.8 Hz, 4H), 3.64 (t, J=5.2 Hz, 2H), 2.80 (bs, 2H), 1.50 (s, 9H).

### Step 6: Synthesis of tert-butyl 4-[1-(4-morpholinopyrido[3,2-d]pyrimidin-2-yl)pyrazol-3-yl]piperidine-1-carboxylate

To a solution of tert-butyl 4-[1-(4-morpholinopyrido[3,2-d]pyrimidin-2-yl)pyrazol-3-yl]-3,6-dihydro-2H-pyridine-1-carboxylate (350 mg, 755 umol) in MeOH (10 mL) was added Pd/C (100 mg, 10% purity) under argon. The resultant mixture hydrogenated under H₂ balloon (˜15 psi) at 25° C. for 12 h. It was then filtered and the filtrate was concentrated in vacuo. The crude product was purified by flash column (ISCO 4 g silica, 20-50% ethyl acetate in petroleum ether, gradient over 20 min) to obtain tert-butyl 4-[1-(4-morpholinopyrido[3,2-d]pyrimidin-2-yl)pyrazol-3-yl]piperidine-1-carboxylate (250 mg) as a pale yellow solid. ¹H NMR (400 MHz, CHLOROFORM-d) δ=8.63 (dd, J=4.1, 1.7 Hz, 1H), 8.50 (d, J=2.4 Hz, 1H), 8.26 (dd, J=8.5, 1.7 Hz, 1H), 7.59 (dd, J=8.5, 4.1 Hz, 1H), 6.30 (d, J=2.6 Hz, 1H), 4.61 (bs, 4H), 4.33-4.09 (m, 2H), 3.97-3.90 (m, 4H), 3.15-3.03 (m, 1H), 2.84 (t, J=12.6 Hz, 2H), 2.07-1.93 (m, 2H), 1.75-1.60 (m, 2H), 1.49 (s, 9H).

### Step 7: Synthesis of 4-[2-[3-(4-piperidyl)pyrazol-1-yl]pyrido[3,2-d]pyrimidin-4-yl]morpholine·HCl

A mixture of tert-butyl 4-[1-(4-morpholinopyrido[3,2-d]pyrimidin-2-yl)pyrazol-3-yl]piperidine-1-carboxylate (130 mg, 0.279 umol) in 4M HCl/EtOAc (10 mL) was stirred at 25° C. for 1 h. The reaction mixture was concentrated to obtain 4-[2-[3-(4-piperidyl)pyrazol-1-yl]pyrido[3,2-d]pyrimidin-4-yl]morpholine·HCl (130 mg, crude) as pale yellow solid.

### Step 8: Synthesis of 1-[4-[1-(4-morpholinopyrido[3,2-d]pyrimidin-2-yl)pyrazol-3-yl]-1-piperidyl]propan-1-one

To a solution of 4-[2-[3-(4-piperidyl)pyrazol-1-yl]pyrido[3,2-d]pyrimidin-4-yl]morpholine·HCl (130 mg, 323 umol) in DCM (3 mL) were added Et₃N (98 mg, 970 umol) and propanoyl chloride (36 mg, 388 umol) at 0° C. The mixture was warmed up and stirred at 20° C. for 1 h and concentrated. The resultant crude product was purified by prep-HPLC (Waters Xbridge Prep OBD C18 150*40 10u column; 30-50% acetonitrile in an a 10 mM ammonium bicarbonate solution in water, 8 min gradient) to obtain 1-[4-[1-(4-morpholinopyrido[3,2-d]pyrimidin-2-yl)pyrazol-3-yl]-1-piperidyl]propan-1-one (71 mg, 52%) as a white solid. ¹H NMR (400 MHz, CHLOROFORM-d) δ=8.63 (dd, J=4.2, 1.8 Hz, 1H), 8.50 (d, J=2.6 Hz, 1H), 8.26 (dd, J=8.6, 1.8 Hz, 1H), 7.59 (dd, J=8.6, 4.2 Hz, 1H), 6.29 (d, J=2.6 Hz, 1H), 4.93-4.24 (m, 5H), 4.00-3.87 (m, 5H), 3.26-3.07 (m, 2H), 2.69 (t, J=11.7 Hz, 1H), 2.39 (q, J=7.5 Hz, 2H), 2.17-1.99 (m, 2H), 1.78-1.65 (m, 2H), 1.18 (t, J=7.5 Hz, 3H). LCMS (ESI) for C22H27N7O2 [M+H]+: 422.3.

### Synthesis of 4-[2-(3-pyrimidin-5-ylpyrazol-1-yl)pyrido[3,2-d]pyrimidin-4-yl]morpholine (Compound 2)

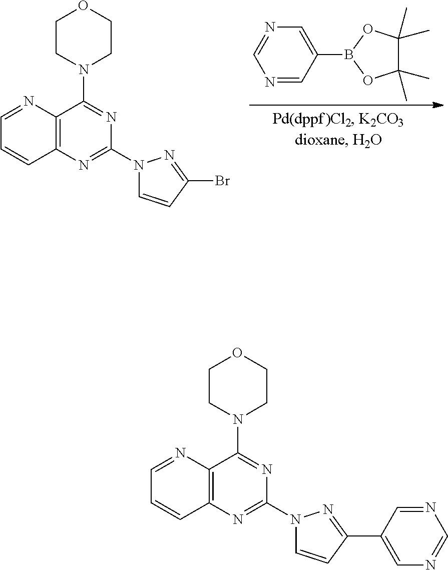 [SMILES: Brc1ccn(-c2nc(N3CCOCC3)c3ncccc3n2)n1 ; CC1(C)OB(c2cncnc2)OC1(C)C ; c1cnc2c(N3CCOCC3)nc(-n3ccc(-c4cncnc4)n3)nc2c1]

To a solution of 4-[2-(3-bromopyrazol-1-yl)pyrido[3,2-d]pyrimidin-4-yl]morpholine (200 mg, 554 umol) in dioxane (5 mL) and H₂O (1 mL) were added 5-(4,4,5,5-tetramethyl-1,3,2-dioxaborolan-2-yl)pyrimidine (171 mg, 831 umol), K₂CO₃ (191 mg, 1.38 mmol) and Pd(dppf)Cl₂ (41 mg, 55 umol) and the resultant mixture was stirred at 60° C. for 12 h under nitrogen. 15 mL of water was added to the mixture and extracted with ethyl acetate (30 mL*2). The combined organic layers were washed with brine (15 mL) and dried over Na₂SO₄. The organic layer was concentrated and the crude product was purified by prep-HPLC (Waters Xbridge Prep OBD C18 150*40 mm*10 um column; 30-65% acetonitrile in an a 10 mM ammonium bicarbonate solution in water, 8 min gradient) to obtain 4-[2-(3-pyrimidin-5-ylpyrazol-1-yl)pyrido[3,2-d]pyrimidin-4-yl]morpholine (14 mg, 38 umol, 7%) as a white solid. 1H NMR (400 MHz, CHLOROFORM-d) δ=9.34 (s, 2H), 9.22 (s, 1H), 8.76-8.67 (m, 2H), 8.27 (dd, J=8.6, 1.5 Hz, 1H), 7.66 (dd, J=8.5, 4.1 Hz, 1H), 6.88 (d, J=2.6 Hz, 1H), 4.9-4.45 (m, 4H), 4.00-3.93 (m, 4H) LCMS (ESI) for C18H16N8O [M+H]+: 361.2

### Synthesis of tert-butyl 3-(1-(4-morpholinopyrido[3,2-d]pyrimidin-2-yl)-1H-pyrazol-3-yl)-5,6-dihydropyridine-1(2H)-carboxylate (Compound 4), tert-butyl 3-(1-(4-morpholinopyrido[3,2-d]pyrimidin-2-yl)-1H-pyrazol-3-yl)piperidine-1-carboxylate (Compound 6), 4-(2-(3-(piperidin-3-yl)-1H-pyrazol-1-yl)pyrido[3,2-d]pyrimidin-4-yl)morpholine (Compound 43) and 4-(2-(3-(1-methylpiperidin-3-yl)-1H-pyrazol-1-yl)pyrido[3,2-d]pyrimidin-4-yl)morpholine (Compound 44)

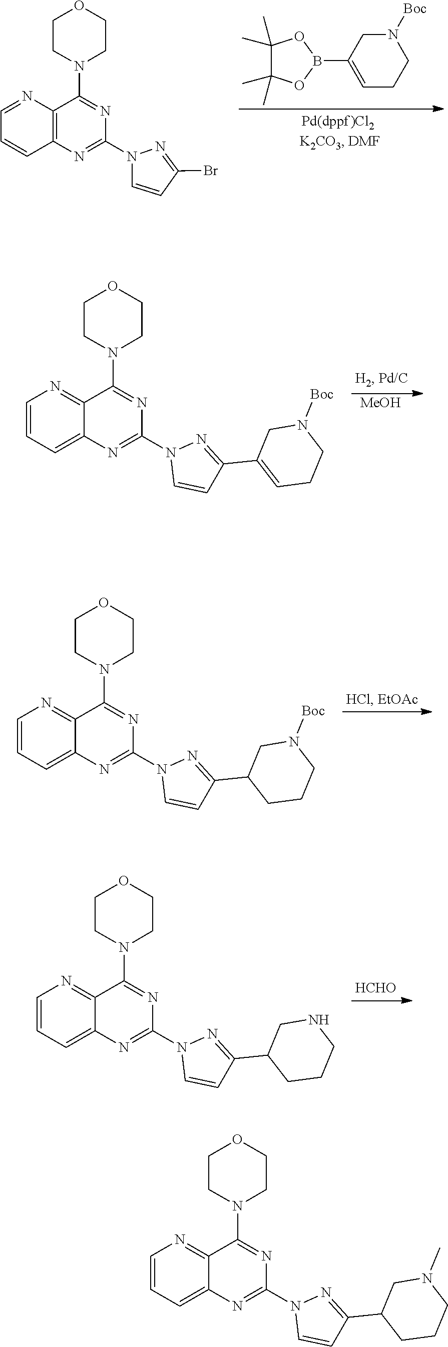

### Step 1: Synthesis of tert-butyl 3-(1-(4-morpholinopyrido[3,2-d]pyrimidin-2-yl)-1H-pyrazol-3-yl)-5,6-dihydropyridine-1(2H)-carboxylate

To a solution of 4-[2-(3-bromopyrazol-1-yl)pyrido[3,2-d]pyrimidin-4-yl]morpholine (400 mg, 1.11 mmol) in dioxane (6 mL) and H₂O (1.2 mL) were added tert-butyl 5-(4,4,5,5-tetramethyl-1,3,2-dioxaborolan-2-yl)-3,6-dihydro-2H-pyridine-1-carboxylate (411 mg, 1.33 mmol), K₂CO₃ (383 mg, 2.77 mmol), and Pd(dppf)Cl₂ (81 mg, 111 umol). The resultant mixture was stirred at 60° C. for 12 h under nitrogen. Then the reaction mixture was diluted with 2 mL H₂O and extracted with EtOAc (3 mL*3). The combined organic phase was dried over anhydrous Na₂SO₄ and concentrated under reduced pressure to give the crude product. It was purified by flash column (ISCO 40 g silica, 40-60% ethyl acetate in petroleum ether, gradient over 20 min) to obtain tert-butyl 5-[1-(4-morpholinopyrido[3,2-d]pyrimidin-2-yl)pyrazol-3-yl]-3,6-dihydro-2H-pyridine-1-carboxylate (480 mg, 94%) as a yellow solid. ¹H NMR (400 MHz, CHLOROFORM-d) δ 8.63 (dd, J=4.1, 1.7 Hz, 1H), 8.54 (d, J=2.6 Hz, 1H), 8.19 (dd, J=8.6, 1.5 Hz, 1H), 7.60 (dd, J=8.6, 4.2 Hz, 1H), 6.53 (bs, 2H), 4.63 (bs, 4H), 4.48 (s, 2H), 3.93 (t, J=4.8 Hz, 4H), 3.58 (t, J=5.4 Hz, 2H), 2.35 (bs, 2H), 1.51 (s, 9H).

LCMS (ESI for C24H29N7O3 [M+H]⁺: 464.2.

### Step 2: Synthesis of tert-butyl 3-(1-(4-morpholinopyrido[3,2-d]pyrimidin-2-yl)-1H-pyrazol-3-yl)piperidine-1-carboxylate

To a solution of tert-butyl 5-[1-(4-morpholinopyrido[3,2-d]pyrimidin-2-yl)pyrazol-3-yl]-3,6-dihydro-2H-pyridine-1-carboxylate (340 mg, 734 umol) in MeOH (3 mL), was added PtO₂ (227 mg, 998 umol) and the resultant mixture was stirred at 25° C. for 1 h under hydrogen atmosphere. The mixture was filtered through celite and the filtrate was concentrated under vacuum. The crude product was purified by prep-HPLC (Waters Xbridge BEH C18 100*25 mm*5 um column; 42%-60% acetonitrile in an a 10 mM ammonium bicarbonate solution, 10 min gradient) to obtain tert-butyl 3-[1-(4-morpholinopyrido[3,2-d]pyrimidin-2-yl)pyrazol-3-yl]piperidine-1-carboxylate (300 mg) as white solid. ¹H NMR (400 MHz, CHLOROFORM-d) δ=8.69-8.58 (m, 1H), 8.50 (d, J=2.4 Hz, 1H), 8.25 (d, J=8.4 Hz, 1H), 7.59 (dd, J=8.5, 4.1 Hz, 1H), 6.33 (d, J=2.6 Hz, 1H), 4.60 (bs, 3H), 4.36-4.03 (m, 2H), 4.01-3.86 (m, 4H), 3.15-3.05 (m, 1H), 3.02-2.92 (m, 1H), 2.83 (bs, 1H), 2.15 (bs, 1H), 1.73 (bs, 1H), 1.62 (bs, 3H), 1.46 (s, 9H). LCMS (ESI for C24H31N7O3 [M+H]⁺: 466.2.

### Step 3: Synthesis of 4-(2-(3-(piperidin-3-yl)-1H-pyrazol-1-yl)pyrido[3,2-d]pyrimidin-4-yl)morpholine

A solution of tert-butyl 3-[1-(4-morpholinopyrido[3,2-d]pyrimidin-2-yl)pyrazol-3-yl]piperidine-1-carboxylate (290 mg, 623 umol) in HCl/EtOAc (8 mL) was stirred at 25° C. for 1 h and concentrated. The mixture was basified by NH₃—H₂O to pH 9 at 0° C. and then it was concentrated again under vacuum. The resultant crude product was purified by prep-HPLC (Luna Omega 5u Polar C18 100A column; 14-36% acetonitrile in an a 0.04% hydrochloric acid solution in water, 7 min gradient) to obtain 4-[2-[3-(3-piperidyl)pyrazol-1-yl]pyrido[3,2-d]pyrimidin-4-yl]morpholine (120 mg) as a white solid. ¹H NMR (400 MHz, DMSO-d6) δ=9.07-8.64 (m, 2H), 8.29-8.17 (m, 1H), 7.82-7.77 (m, 1H), 6.53 (d, J=2.6 Hz, 1H), 4.57 (bs, 4H), 3.84 (t, J=4.5 Hz, 4H), 3.47-3.37 (m, 1H), 3.35-3.23 (m, 2H), 3.18-3.06 (m, 1H), 2.94 (bs, 1H), 2.18-2.06 (m, 1H), 1.96-1.84 (m, 2H), 1.78-1.67 (m, 1H). LCMS (ESI for C19H23N7O [M+H]⁺: 366.2.

### Step 4: Synthesis of 4-(2-(3-(1-methylpiperidin-3-yl)-1H-pyrazol-1-yl)pyrido[3,2-d]pyrimidin-4-yl)morpholine

To a solution of 4-[2-[3-(3-piperidyl)pyrazol-1-yl]pyrido[3,2-d]pyrimidin-4-yl]morpholine (120 mg, 328 umol) in HCHO (2 mL), were added CH3COOH (20 mg, 328 umol) and NaBH₃CN (21 mg, 328 umol, 1 eq) at 0° C., then the mixture was stirred at 20° C. for 12 h. The resultant reaction mixture was concentrated and the crude product was purified by prep-HPLC (Phenomenex gemini-NX C18 75*30 mm*3 um column; 15%-45% acetonitrile in an a 0.04% ammonium hydroxide and 10 mM ammonium bicarbonate solution, 10 min gradient) to obtain 4-[2-[3-(1-methyl-3-piperidyl)pyrazol-1-yl]pyrido[3,2-d]pyrimidin-4-yl]morpholine (29 mg, 23%) as a pale yellow solid. ¹H NMR (400 MHz, CHLOROFORM-d) δ 8.61 (dd, J=4.2, 1.7 Hz, 1H), 8.48 (d, J=2.9 Hz, 1H), 8.24 (dd, J=8.6, 1.7 Hz, 1H), 7.57 (dd, J=8.6, 4.2 Hz, 1H), 6.31 (d, J=2.4 Hz, 1H), 4.59 (bs, 4H), 4.03-3.66 (m, 4H), 3.35-3.16 (m, 2H), 2.89-2.80 (m, 1H), 2.20-2.05 (m, 3H), 2.00-1.99 (m, 2H), 1.98-1.95 (m, 1H), 1.86-1.71 (m, 2H), 1.56-1.37 (m, 1H). LCMS (ESI for C20H25N7O [M+H]⁺: 380.2.

### Synthesis of 4-(2-indazol-1-ylpyrido[3,2-d]pyrimidin-4-yl)morpholine (Compound 53) and 4-(2-indazol-2-ylpyrido[3,2-d]pyrimidin-4-yl)morpholine (Compound 7)

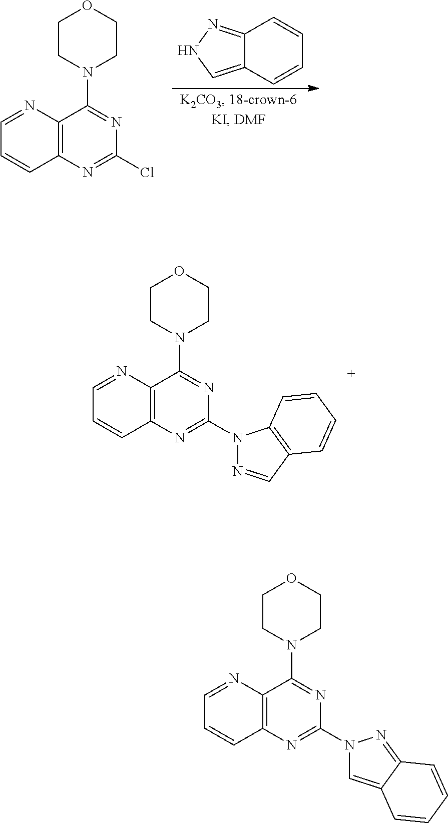 [SMILES: Clc1nc(N2CCOCC2)c2ncccc2n1 ; c1ccc2n[nH]cc2c1 ; c1ccc2c(c1)cnn2-c1nc(N2CCOCC2)c2ncccc2n1 ; c1ccc2nn(-c3nc(N4CCOCC4)c4ncccc4n3)cc2c1]

To a solution of 2H-indazole (80 mg, 677 umol) and 4-(2-chloropyrido[3,2-d]pyrimidin-4-yl)morpholine (221 mg, 880 umol) in DMF (1 mL) were added 18-CROWN-6 (1 mg, 5 umol), K₂CO₃ (133 mg, 965 mol) and KI (5 mg, 33 umol) and the resultant mixture was heated at 130° C. for 5 h. The mixture was filtered and the crude products from the filtrate were purified by prep-HPLC (Waters Xbridge BEH C18 100*25 mm*5 um column; 30-60% acetonitrile in an 10 mM ammonium bicarbonate in water, 10 min gradient) to obtain 4-(2-indazol-2-ylpyrido[3,2-d]pyrimidin-4-yl)morpholine (31 mg, 14%) and 4-(2-indazol-1-ylpyrido[3,2-d]pyrimidin-4-yl)morpholine (118 mg, 52%) as pale yellow solids.

Compound 7: ¹H NMR (400 MHz, DMSO-d6) δ 8.80 (d, J=8.4 Hz, 1H), 8.73 (d, J=3.6 Hz, 1H), 8.43 (s, 1H), 8.22 (d, J=8.4 Hz, 1H), 7.89 (d, J=8 Hz, 1H), 7.81 (dd, J=8.0, 3.6 Hz, 1H), 7.58 (t, J=8 Hz, 1H), 7.33 (t, J=7.6 Hz, 1H), 4.57 (bs, 4H), 3.85 (t, J=4.0 Hz, 4H). LCMS (ESI for C18H16N6O [M+H]+: 333.2.

Compound 53: ¹H NMR (400 MHz, DMSO-d6) δ 9.33 (s, 1H), 8.80 (d, J=2.8 Hz, 1H), 8.24 (d, J=8.4 Hz, 1H), 7.86 (dd, J=8.4, 4.8 Hz, 1H), 7.78 (d, J=8.4 Hz, 1H), 7.72 (d, J=8.8 Hz, 1H), 7.33 (t, J=8.8 Hz, 1H), 7.11 (t, J=8.4 Hz, 1H), 4.57 (bs, 4H), 3.85 (t, J=4.8 hz, 4H). LCMS (ESI for C18H16N6O [M+H]+: 333.2.

### Synthesis of 4-[2-(4-phenylpyrazol-1-yl)-7-tetrahydropyran-4-yl-pyrido[3,2-d]pyrimidin-4-yl]morpholine (Compound 63)

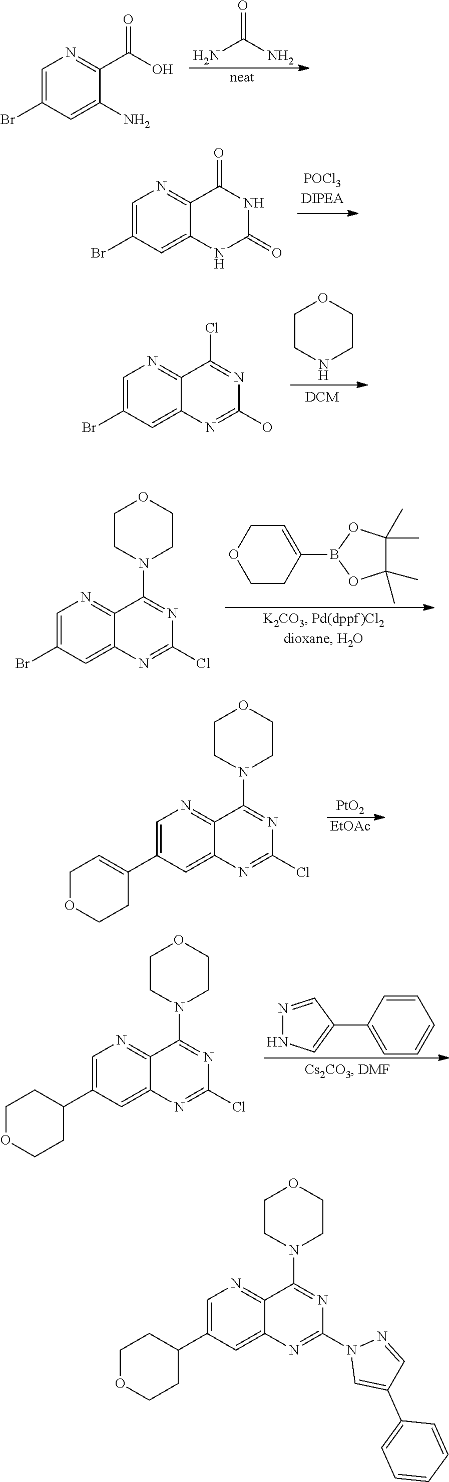

### Step 1: Synthesis of 7-bromo-1H-pyrido[3,2-d]pyrimidine-2,4-dione

A mixture of 3-amino-5-bromo-pyridine-2-carboxylic acid (5 g, 23.4 mmol) and urea (2.77 g, 46.08 mmol) was heated with stirring in a flask at 20000 for 2 h. The mixture was cooled, water (100 mL) and MeOH (10 mL) were added to the flask and stirred. The resultant precipitate was filtered and dried obtain 7-bromo-1H-pyrido[3,2-d]pyrimidine-2,4-dione (4.6 g, 82%) as brown solid. ¹H NMR (400 MHz, DMSO-d6) b=11.55 (bs, 1H), 11.26 (b, 1H), 8.52 (d, J=2.2 Hz, 1H), 7.73 (d, J=2.2 Hz, 1H).

### Step 2: Synthesis of 7-bromo-2,4-dichloro-pyrido[3,2-d]pyrimidine

To a mixture of 7-bromo-12H-pyrido[3,2-d]pyrimidine-2,4-dione (3 g, 12.48 mmol) in PO₃ (25 mL) was added DIPEA (3.20 g, 24.79 mmol). The mixture was stirred at 120° C. for 1 h and concentrated. To the residue 30 mL ice water was added and stirred at 20° C. for 0.5 h and it was basified with 2N NaOH (30 m). The resultant mixture was extracted with DCM (50 mL*2), washed with brine (30 m), dried over Na₂SO₄ and concentrated to obtain 7-bromo-2,4-dichloro-pyrido[3,2-d]pyrimidine (3 g) as brown solid. H NMR (400 MHz, CHLOROFORM-d) δ=9.11 (d, J=2.1 Hz, 1H), 8.49 (d, J=2.1 Hz, 1H) Step 3: Synthesis of 4-(7-bromo-2-chloro-pyrido[3,2-d]pyrimidin-4-yl)morpholine.

To a solution of 7-bromo-2,4-dichloro-pyrido[3,2-d]pyrimidine (2.7 g, 9.68 mmol) in THE (50 mL) were added Et₃N (1.08 g, 10.65 mmol) and morpholine (928 mg, 10.65 mmol) at 0° C. The mixture was warmed up and stirred at 20° C. for 1 h and concentrated. The residue was dissolved in 100 mL chloroform, washed with saturated aqueous solution of sodium bicarbonate, dried over Na₂SO₄ and concentrated. The crude product was purified by flash column (ISCO 50 g silica, 0-50% ethyl acetate in petroleum ether, gradient over 20 min) to obtain 4-(7-bromo-2-chloro-pyrido[3,2-d]pyrimidin-4-yl)morpholine (2.5 g) as yellow solid.

### Step 4: Synthesis of 4-[2-chloro-7-(3,6-dihydro-2H-pyran-4-yl)pyrido[3,2-d]pyrimidin-4-yl]morpholine

To a solution of 4-(7-bromo-2-chloro-pyrido[3,2-d]pyrimidin-4-yl)morpholine (2.4 g, 7.28 mmol) in dioxane (30 mL) and H₂O (6 mL) were added 2-(3,6-dihydro-2H-pyran-4-yl)-4,4,5,5-tetramethyl-1,3,2-dioxaborolane (1.53 g, 7.28 mmol), K₂CO₃ (2.52 g, 18.20 mmol) and Pd(dppf)Cl₂ (266 mg, 364 umol) and the resultant stirred at 80° C. for 2 h under argon atmosphere. 30 mL of water was added to the reaction mixture and extracted with ethyl acetate (50 mL*2). The combined organic layers were washed with brine (30 mL), dried over Na₂SO₄ and concentrated. The crude product was purified by flash column (ISCO 20 g silica, 20-50% ethyl acetate in petroleum ether, gradient over 30 min) to obtain 4-[2-chloro-7-(3,6-dihydro-2H-pyran-4-yl)pyrido[3,2-d]pyrimidin-4-yl]morpholine (0.9 g, 2.70 mmol, 37%) as pale yellow solid.

### Step 5: Synthesis of 4-(2-chloro-7-tetrahydropyran-4-yl-pyrido[3,2-d]pyrimidin-4-yl)morpholine

To a solution of 4-[2-chloro-7-(3,6-dihydro-2H-pyran-4-yl)pyrido[3,2-d]pyrimidin-4-yl]morpholine (850 mg, 2.55 mmol) in EtOAc (20 mL) and DCM (20 mL) was added PtO₂ (580 mg, 2.55 mmol) under argon. The suspension was degassed under vacuum and purged with H₂ several times and further stirred under hydrogen balloon (15 psi) at 20° C. for 20 min. The mixture was then filtered and the filtrate was concentrated to obtain 4-(2-chloro-7-tetrahydropyran-4-yl-pyrido[3,2-d]pyrimidin-4-yl)morpholine (890 mg, crude) as pale yellow solid.

### Step 6: Synthesis of 4-[2-(4-phenylpyrazol-1-yl)-7-tetrahydropyran-4-yl-pyrido[3,2-d]pyrimidin-4-yl]morpholine

To a solution of 4-(2-chloro-7-tetrahydropyran-4-yl-pyrido[3,2-d]pyrimidin-4-yl)morpholine (0.1 g, 299 umol) in DMF (2 mL) were added 4-phenyl-1H-pyrazole (47 mg, 329 umol) and Cs₂CO₃ (195 mg, 597 umol). The resultant mixture was stirred at 100° C. for 16 h followed by the addition of 15 mL of water. The mixture was then extracted with ethyl acetate (30 mL*2), washed with brine (15 mL), dried over Na₂SO₄ and concentrated. The resultant crude product was purified by prep-HPLC (Phenomenex gemini-NX 150*30 5u column; 30-60% acetonitrile in an a 10 mM ammonium bicarbonate solution in water, 8 min gradient) to obtain 4-[2-(4-phenylpyrazol-1-yl)-7-tetrahydropyran-4-yl-pyrido[3,2-d]pyrimidin-4-yl]morpholine (65 mg, 49%) as pale yellow solid. ¹H NMR (400 MHz, CHLOROFORM-d) δ=8.79 (s, 1H), 8.56 (d, J=2.3 Hz, 1H), 8.12 (s, 1H), 8.06 (d, J=2.1 Hz, 1H), 7.67-7.60 (m, 2H), 7.43 (t, J=7.7 Hz, 2H), 7.34-7.27 (m, 1H), 4.65 (bs, 4H), 4.23-4.09 (m, 2H), 4.02-3.88 (m, 4H), 3.67-3.54 (m, 2H), 3.07-2.90 (m, 1H), 2.00-1.84 (m, 4H). LCMS (ESI) for C25H26N6O2 [M+H]+: 443.3.

The following compounds were synthesized according to the protocol described above:

| Name | Structure | NMR, MS | # |
|---|---|---|---|
| 4-(2-(3-phenyl- 1H-pyrazol-1- yl)pyrido[3,2- d]pyrimidin-4- yl)morpholine | 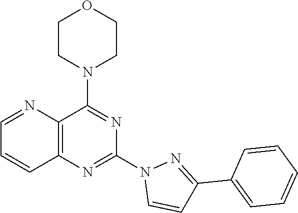 [SMILES: c1ccc(-c2ccn(-c3nc(N4CCOCC4)c4ncccc4n3)n2)cc1] | ¹H NMR (400 MHz, DMSO-d₆) δ 8.80 (d, J = 2.7 Hz, 1H), 8.76 (dd, J = 4.1, 1.7 Hz, 1H), 8.21 (dd, J = 8.5, 1.7 Hz, 1H), 8.01 − 7.95 (m, 2H), 7.83 (dd, J = 8.5, 4.1 Hz, 1H), 7.49 (t, J = 7.5 Hz, 2H), 7.40 (t, J = 7.3 Hz, 1H), 7.09 (d, J = 2.7 Hz, 1H), 4.58 (s, 4H), 3.83 (t, J = 8 Hz, 4H). LCMS (ESI) m/z: 359.3 [M + H]⁺. | 8 |
| 4-(2-(3-(pyridin- 2-yl)-1H- pyrazol-1- yl)pyrido[3,2- d]pyrimidin-4- yl)morpholine | 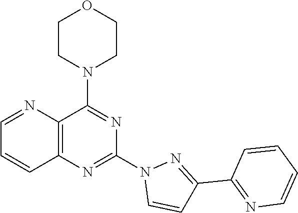 [SMILES: c1ccc(-c2ccn(-c3nc(N4CCOCC4)c4ncccc4n3)n2)nc1] | ¹H NMR (400 MHz, DMSO-d₆) δ 8.82 (d, J = 2.7 Hz, 1H), 8.77 (dd, J = 4.1, 1.7 Hz, 1H), 8.70 − 8.62 (m, 1H), 8.23 (dd, J = 8.5, 1.7 Hz, 1H), 8.15 (d, J = 7.9 Hz, 1H), 7.93 (td, J = 7.7, 1.8 Hz, 1H), 7.84 (dd, J = 8.5, 4.1 Hz, 1H), 7.41 (ddd, J = 7.5, 4.8, 1.1 Hz, 1H), 7.12 (d, J = 2.7 Hz, 1H), 4.60 (s, 4H), 3.84 (t, J = 8.0 Hz, 4H). LCMS (ESI) m/z: 360.1 [M + H]⁺. | 12 |
| 4-(2-(3-(pyridin- 3-yl)-1H- pyrazol-1- yl)pyrido[3,2- d]pyrimidin-4- yl)morpholine | 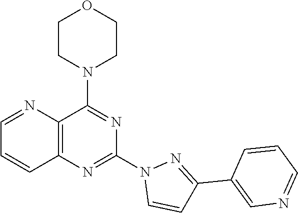 [SMILES: c1cncc(-c2ccn(-c3nc(N4CCOCC4)c4ncccc4n3)n2)c1] | ¹H NMR (400 MHz, DMSO-d₆) δ 9.24 − 9.13 (m, 1H), 8.84 (d, J = 2.7 Hz, 1H), 8.76 (dd, J = 4.1, 1.7 Hz, 1H), 8.60 (dd, J = 4.8, 1.6 Hz, 1H), 8.35 (dt, J = 8.0, 1.6 Hz, 1H), 8.21 (dd, J = 8.5, 1.7 Hz, 1H), 7.83 (dd, J = 8.5, 4.1 Hz, 1H), 7.52 (ddd, J = 8.0, 4.8, 0.8 Hz, 1H), 7.20 (d, J = 2.7 Hz, 1H), 4.60 (bs, 4H), 3.92 − 3.77 (m, 4H). LCMS (ESI) m/z: 360.1 [M + H]⁺. | 11 |
| 4-(2-(3-(pyridin- 4-yl)-1H- pyrazol-1- yl)pyrido[3,2- d]pyrimidin-4- yl)morpholine | 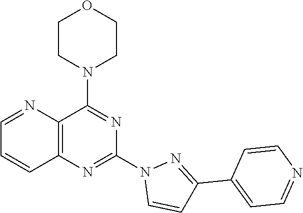 [SMILES: c1cnc2c(N3CCOCC3)nc(-n3ccc(-c4ccncc4)n3)nc2c1] | ¹H NMR (400 MHz, DMSO-d₆) δ 8.87 (d, J = 2.7 Hz, 1H), 8.78 (dd, J = 4.1, 1.7 Hz, 1H), 8.68 (dd, J = 4.5, 1.6 Hz, 2H), 8.23 (dd, J = 8.5, 1.7 Hz, 1H), 7.94 (dd, J = 4.5, 1.6 Hz, 2H), 7.84 (dd, J = 8.5, 4.2 Hz, 1H), 7.25 (d, J = 2.7 Hz, 1H), 4.59 (bs, 4H), 3.90 − 3.80 (m, 4H). LCMS (ESI) m/z: 360.1 [M + H]⁺. | 10 |
| 4-[2-(3- phenylpyrazol- 1-yl)-7- tetrahydropyran- 4-yl- pyrido[3,2- d]pyrimidin-4- yl]morpholine | 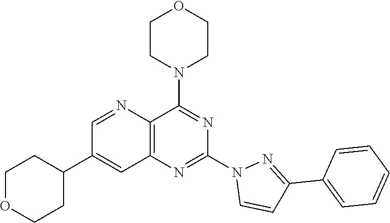 [SMILES: c1ccc(-c2ccn(-c3nc(N4CCOCC4)c4ncc(C5CCOCC5)cc4n3)n2)cc1] | ¹H NMR (400 MHz, CHLOROFORM-d) δ = 9.57 (s, 1H), 8.76 (bs, 1H), 8.64 (d, J = 1.6 Hz, 1H), 8.08 (bs d, J = 6.8 Hz, 2H), 7.34 − 7.24 (m, 3H), 6.97 (bs, 1H), 5.43 − 4.22 (m, 4H), 4.20 − 4.11 (m, 2H), 3.98 (bs, 4H), 3.60 (dt, J = 11.4, 2.3 Hz, 2H), 3.23 − 3.06 (m, 1H), 2.10 − 1.87 (m, 4H). LCMS (ESI) for C25H26N602 [M + H]+: 443.2. | 20 |
| 4-(2-(3- isopropyl-1H- pyrazol-1- yl)pyrido[3,2- d]pyrimidin-4- yl)morpholine | 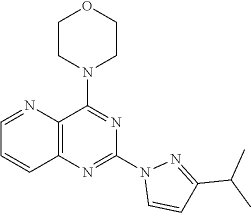 [SMILES: CC(C)c1ccn(-c2nc(N3CCOCC3)c3ncccc3n2)n1] | ¹H NMR (400 MHz, DMSO-d₆) δ 8.72 (dd, J = 4.1, 1.6 Hz, 1H), 8.60 (d, J = 2.6 Hz, 1H), 8.15 (dd, J = 8.5, 1.6 Hz, 1H), 7.80 (dd, J = 8.5, 4.1 Hz, 1H), 6.43 (d, J = 2.6 Hz, 1H), 4.50 (bs, 4H), 3.81 (t, J = 4 Hz, 4H), 3.04 − 2.99 (m, 1H), 1.26 (d, J = 6.9 Hz, 6H). LCMS (ESI) m/z: 325.2 [M + H]⁺. | 9 |
| 4-[2-[3-(2- fluorophenyl) pyrazol-1-yl]-7- tetrahydropyran- 4-yl-pyrido[3,2- d]pyrimidin-4- yl]morpholine | 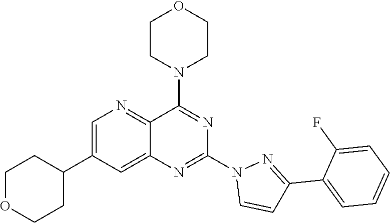 [SMILES: Fc1ccccc1-c1ccn(-c2nc(N3CCOCC3)c3ncc(C4CCOCC4)cc3n2)n1] | ¹H NMR (400 MHz, CHLOROFORM-d) δ = 8.65 (d, J = 2.6 Hz, 1H), 8.56 (d, J = 2.1 Hz, 1H), 8.33 (dt, J = 7.7, 1.4 Hz, 1H), 8.08 (d, J = 2.1 Hz, 1H), 7.38 − 7.30 (m, 1H), 7.26 − 7.21 (m, 1H), 7.15 (dd, J = 11.1, 8.4 Hz, 1H), 7.00 − 6.93 (m, 1H), 4.65 (bs, 4H), 4.23 − 4.08 (m, 2H), 3.93 (t, J = 4.4 Hz, 4H), 3.60 (dt, J = 11.2, 3.1 Hz, 2H), 3.04 − 2.90 (m, 1H), 1.99 − 1.84 (m, 4H). LCMS (ESI) for C25H25FN6O2 [M + H]+: 461.3 | 67 |
| 4-[2-[3-(4- fluorophenyl) pyrazol-1-yl]-7- tetrahydropyran- 4-yl- pyrido[3,2- d]pyrimidin-4- yl]morpholine | 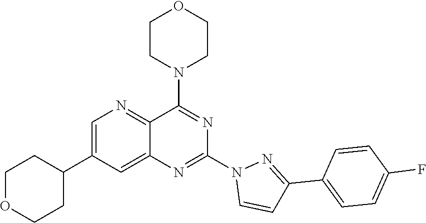 [SMILES: Fc1ccc(-c2ccn(-c3nc(N4CCOCC4)c4ncc(C5CCOCC5)cc4n3)n2)cc1] | ¹H NMR (400 MHz, CHLOROFORM-d) δ = 8.63 (d, J = 2.8 Hz, 1H), 8.55 (d, J = 2.1 Hz, 1H), 8.07 (d, J = 2.1 Hz, 1H), 8.04 − 7.96 (m, 2H), 7.13 (t, J = 8.7 Hz, 2H), 6.76 (d, J = 2.8 Hz, 1H), 4.64 (bs, 4H), 4.15 (dd, J = 10.4, 2.8 Hz, 2H), 3.94 (t, J = 4.8 Hz, 4H), 3.60 (dt, J = 11.2, 3.2 Hz, 2H), 3.05 − 2.92 (m, 1H), 1.98 − 1.82 (m, 4H). LCMS (ESI) for C25H25FN6O2 [M + H]+: 461.2. | 68 |
| 4-[2-[3-(2- pyridyl)pyrazol- 1-yl]-7- tetrahydropyran- 4-yl- pyrido[3,2- d]pyrimidin-4- yl]morpholine | 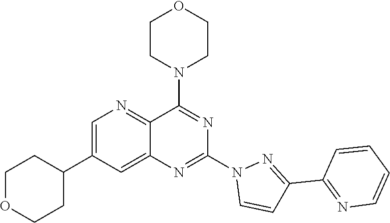 [SMILES: c1ccc(-c2ccn(-c3nc(N4CCOCC4)c4ncc(C5CCOCC5)cc4n3)n2)nc1] | ¹H NMR (400 MHz, CHLOROFORM-d) δ = 8.71 − 8.63 (m, 2H), 8.56 (d, J = 2.3 Hz, 1H), 8.38 (d, J = 8.0 Hz, 1H), 8.08 (d, J = 2.0 Hz, 1H), 7.79 (dt, J = 1.7, 7.7 Hz, 1H), 7.29 (d, J = 0.8 Hz, 1H), 7.20 (d, J = 2.6 Hz, 1H), 4.66 (bs, 4H), 4.16 (dd, J = 10.4, 2.8 Hz, 2H), 4.03 − 3.89 (m, 4H), 3.61 (dt, J = 11.2, 3.1 Hz, 2H), 3.08 − 2.93 (m, 1H), 2.03 − 1.81 (m, 4H). LCMS (ESI) for C24H25N7O2 [M + H]+: 444.3. | 60 |

### Synthesis of 4-(7-Ethyl-2-(3-(pyridin-4-yl)-1H-pyrazol-1-yl)pyrido[3,2-d]pyrimidin-4-yl)morpholine (Compound 14)

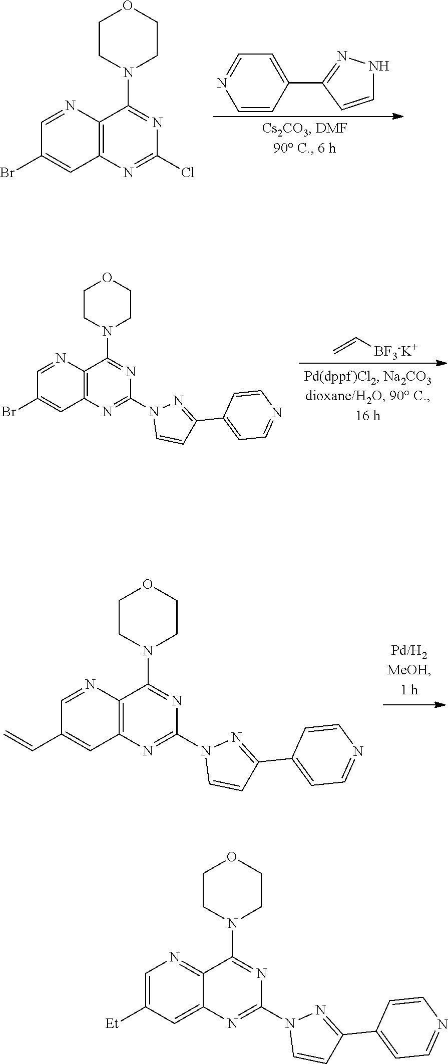

### Step 1: Synthesis of 4-(7-Bromo-2-(3-(pyridin-4-yl)-1H-pyrazol-1-yl)pyrido[3,2-d]pyrimidin-4-yl)morpholine

A mixture of 4-(7-bromo-2-chloropyrido[3,2-d]pyrimidin-4-yl)morpholine (160 mg, 0.49 mmol), 4-(1H-pyrazol-3-yl)pyridine (92 mg, 0.63 mmol) and cesium carbonate (319 mg, 0.98 mmol) in N,N-dimethylformamide (5 mL) was heated to 80° C. and stirred for 6 h. Then the reaction was quenched by the addition water (25 mL) and was extracted with dichloromethane (25 ml*3). The organic layer was dried over Na₂SO₄, filtered and concentrated. The obtained residue was purified by flash chromatography on silica gel (petroleum ether:ethyl acetate=4:1) to obtain 4-(7-bromo-2-(3-(pyridin-4-yl)-1H-pyrazol-1-yl)pyrido[3,2-d]pyrimidin-4-yl)morpholine (120 mg, 56%) as yellow solid. LCMS (ESI) m/z: 438.0[M+H]⁺.

### Step 2: Synthesis of 4-(2-(3-(Pyridin-4-yl)-1H-pyrazol-1-yl)-7-vinylpyrido[3,2-d]pyrimidin-4-yl)morpholine

A solution of 4-(7-bromo-2-(3-(pyridin-4-yl)-1H-pyrazol-1-yl)pyrido[3,2-d]pyrimidin-4-yl)morpholine (120 mg, 0.27 mmol), potassium vinyltrifluoroborate (72 mg, 0.54 mmol), 1,1′-bis(diphenylphosphino)ferrocene-palladium(II)dichloride (20 mg, 0.027 mmol) and sodium carbonate (67 mg, 0.54 mmol) in water (2.0 mL) and dioxane (4.0 mL) was stirred at 90° C. for 1 h under argon atmosphere. The mixture was then diluted with ethyl acetate (50 mL) and washed with water (25 mL). The organic layer was concentrated and purified by purified by flash chromatography (dichloromethane:methanol=20:1) to obtain 4-(2-(3-(pyridin-4-yl)-1H-pyrazol-1-yl)-7-vinylpyrido[3,2-d]pyrimidin-4-yl)morpholine (80 mg, 77%) as white solid. LCMS (ESI) m/z: 386.3 [M+H]⁺.

### Step 3: Synthesis of 4-(7-Ethyl-2-(3-(pyridin-4-yl)-1H-pyrazol-1-yl)pyrido[3,2-d]pyrimidin-4-yl)morpholine

Palladium on carbon (10 mg, 10% loading) was added to a solution of 4-(2-(3-(pyridin-4-yl)-1H-pyrazol-1-yl)-7-vinylpyrido[3,2-d]pyrimidin-4-yl)morpholine (80 mg, 0.21 mmol) in methanol (5 ml) and the resultant mixture was stirred at 20° C. for 1 h under hydrogen atmosphere. The mixture was then filtered, concentrated and the obtained residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate) to obtain 4-(7-ethyl-2-(3-(pyridin-4-yl)-1H-pyrazol-1-yl)pyrido[3,2-d]pyrimidin-4-yl)morpholine (24.3 mg, 30%) as white solid. ¹H NMR (400 MHz, DMSO-d6) δ 8.85 (d, J=2.0 Hz, 1H), 8.69-8.67 (m, 3H), 8.02 (d, J=2.0 Hz, 1H), 7.94-7.92 (m, 2H), 7.24 (d, J=2.0 Hz, 1H), 4.59 (bs, 4H), 3.82 (t, J=4.2 Hz, 4H), 2.86 (q, J=6.0 Hz, 2H), 1.32 (t, J=6.0 Hz, 3H); LCMS (ESI) m/z: 388.0 [M+H]⁺.

### Synthesis of 7-(furan-2-yl)-4-morpholino-N-phenylpyrido[3,2-d]pyrimidin-2-amine (Compound 15)

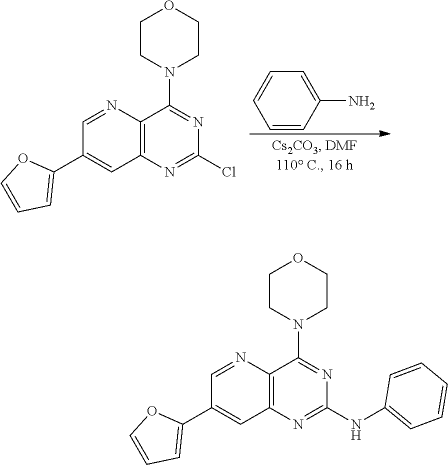 [SMILES: Clc1nc(N2CCOCC2)c2ncc(-c3ccco3)cc2n1 ; Nc1ccccc1 ; c1ccc(Nc2nc(N3CCOCC3)c3ncc(-c4ccco4)cc3n2)cc1]

To a solution of 4-(2-chloro-7-(furan-2-yl)pyrido[3,2-d]pyrimidin-4-yl)morpholine (200 mg, 0.63 mmol) and aniline (65 mg, 0.69 mmol) in DMF (7 mL) was added Cs₂CO₃ (619 mg, 1.90 mmol). The reaction mixture was stirred at 110° C. for 16 h and concentrated. The residue was subjected to prep-HPLC (0.05% FA/H2O:CH3CN=5%-95%) to obtain 7-(furan-2-yl)-4-morpholino-N-phenylpyrido[3,2-d]pyrimidin-2-amine (35 mg, P: 100%, Y: 11%) as a yellow solid. ¹H NMR (400 MHz, DMSO-d6) δ 9.28 (s, 1H), 8.86 (d, J=2.4 Hz, 1H), 7.98 (d, J=2.4 Hz, 1H), 7.93 (d, J=1.6 Hz, 1H), 7.89 (d, J=8.4 Hz, 2H), 7.37 (d, J=3.2 Hz, 1H), 7.29 (t, J=8.0 Hz, 2H), 6.94 (t, J=8.0 Hz, 1H), 6.74-6.72 (m, 1H), 4.40 (bs, 4H), 3.80 (t, J=4.4 Hz, 4H); LCMS (ESI) m/z: 374.3 [M+H]+.

### Synthesis of 4-(7-(furan-2-yl)-2-phenoxypyrido[3,2-d]pyrimidin-4-yl)morpholine (Compound 16)

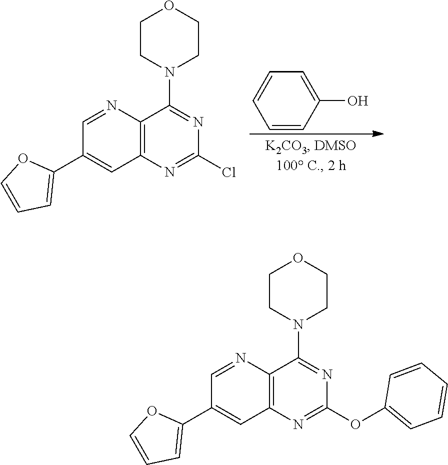 [SMILES: Clc1nc(N2CCOCC2)c2ncc(-c3ccco3)cc2n1 ; Oc1ccccc1 ; c1ccc(Oc2nc(N3CCOCC3)c3ncc(-c4ccco4)cc3n2)cc1]

To a solution of 4-(2-chloro-7-(furan-2-yl)pyrido[3,2-d]pyrimidin-4-yl)morpholine (200 mg, 0.63 mmol) and phenol (65 mg, 0.69 mmol) in DMF (8 mL) was added K₂CO₃ (262 mg, 1.90 mmol). The reaction mixture was stirred at 100° C. for 2 h and concentrated. The residue was purified by prep-HPLC (0.05% FA/H2O:CH3CN=5%-95%) to afford 4-(7-(furan-2-yl)-2-phenoxypyrido[3,2-d]pyrimidin-4-yl)morpholine (40 mg, 17%) as yellow solid. ¹H NMR (400 MHz, DMSO-d6) δ 9.02 (d, J=2.4 Hz, 1H), 8.01 (d, J=2.4 Hz, 1H), 7.93 (s, 1H), 7.47-7.41 (m, 3H), 7.25 (t, J=8.4 Hz, 3H), 6.72 (dd, J=3.2, 1.6 Hz, 1H), 4.37 (bs, 4H), 3.76 (t, J=4.8 Hz, 4H); LCMS (ESI) m/z: 375.1 [M+H]+.

### Synthesis of 4-(7-bromo-2-(3-phenyl-1H-pyrazol-1-yl)pyrido[3,2-d]pyrimidin-4-yl)morpholine (Compound 17)

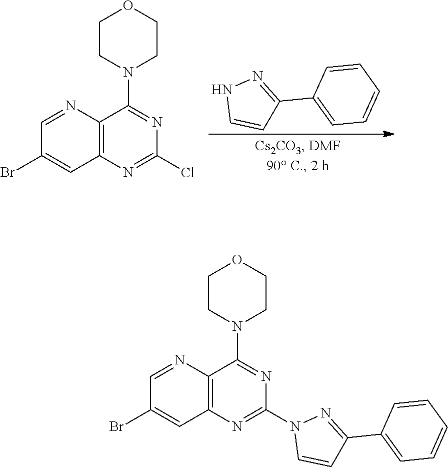 [SMILES: Clc1nc(N2CCOCC2)c2ncc(Br)cc2n1 ; c1ccc(-c2cc[nH]n2)cc1 ; Brc1cnc2c(N3CCOCC3)nc(-n3ccc(-c4ccccc4)n3)nc2c1]

### Step 1: Synthesis of 4-(7-bromo-2-(3-phenyl-1H-pyrazol-1-yl)pyrido[3,2-d]pyrimidin-4-yl)morpholine

A mixture of 4-(7-bromo-2-chloropyrido[3,2-d]pyrimidin-4-yl)morpholine (500 mg, 1.52 mmol), cesium carbonate (990 mg, 3.03 mmol) and 3-phenyl-1H-pyrazole (0.26 mg, 1.82 mmol) in DMF (10 mL) was stirred at 90° C. for 2 h. The resultant crude product was purified by silica gel column chromatography (petroleum ether:acetic ester=2:1) to obtain the target product (450 mg, 68%) as yellow solid. ¹H NMR (400 MHz, DMSO-d6) δ 8.77 (dd, J=8.0, 2.4 Hz, 2H), 8.48 (d, J=2.0 Hz, 1H), 7.99-7.96 (m, 2H), 7.52-7.38 (m, 3H), 7.09 (d, J=2.7 Hz, 1H), 4.60 (bs, 4H), 3.83 (t, J=4.4 Hz, 4H); LCMS (ESI) m/z: 438.9 [M+H]⁺.

### Synthesis of 4-(2-(3-Phenyl-1H-pyrazol-1-yl)-7-(tetrahydro-2H-pyran-2-yl)pyrido[3,2-d]pyrimidin-4-yl)morpholine (Compound 18)

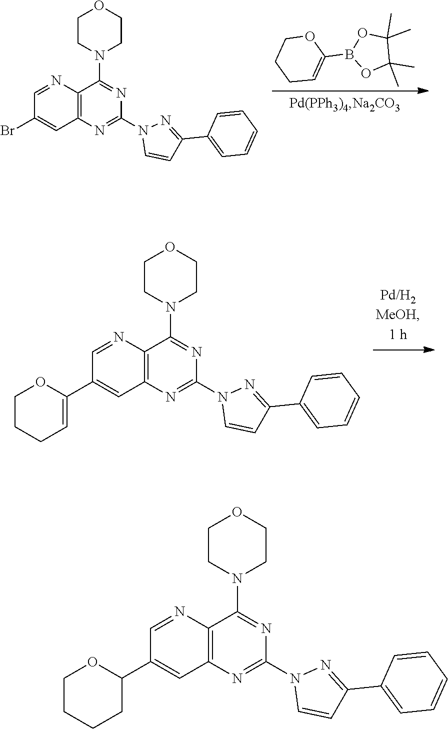 [SMILES: Brc1cnc2c(N3CCOCC3)nc(-n3ccc(-c4ccccc4)n3)nc2c1 ; CC1(C)OB(C2=CCCCO2)OC1(C)C ; C1=C(c2cnc3c(N4CCOCC4)nc(-n4ccc(-c5ccccc5)n4)nc3c2)OCCC1 ; c1ccc(-c2ccn(-c3nc(N4CCOCC4)c4ncc(C5CCCCO5)cc4n3)n2)cc1]

### Step 1: Synthesis of 4-(7-(3,4-Dihydro-2H-pyran-6-yl)-2-(3-phenyl-1H-pyrazol-1-yl)pyrido[3,2-d]pyrimidin-4-yl)morpholine

A solution of 4-(7-Bromo-2-(3-phenyl-1H-pyrazol-1-yl)pyrido[3,2-d]pyrimidin-4-yl)morpholine (100 mg, 0.23 mmol), 2-(3,4-dihydro-2H-pyran-6-yl)-4,4,5,5-tetramethyl-1,3,2-dioxaborolane (97 mg, 0.46 mmol), tetrakis(triphenylphosphine)palladium (23 mg, 0.023 mmol) and sodium carbonate (29 mg, 0.28 mmol) in water (0.5 mL) and dioxane (2.0 mL) was stirred at 80° C. for 4 h under argon atmosphere. Water (25 mL) was added to the mixture and then it was extracted with dichloromethane (25 ml*3). The combined organic layer was dried on Na₂SO₄, filtered and concentrated. The obtained residue was subjected to silica gel column chromatography (petroleum ether/ethyl acetate=4:1) to obtain 4-(7-(3,4-dihydro-2H-pyran-6-yl)-2-(3-phenyl-1H-pyrazol-1-yl)pyrido[3,2-d]pyrimidin-4-yl)morpholine (60 mg, 60%) as yellow solid. LCMS (ESI) m/z: 441.1 [M+H]⁺.

### Step 2: Synthesis of 4-(2-(3-Phenyl-1H-pyrazol-1-yl)-7-(tetrahydro-2H-pyran-2-yl)pyrido[3,2-d]pyrimidin-4-yl)morpholine

Palladium on carbon (4 mg, 10% loading) was added to a solution of 4-(7-(3,4-dihydro-2H-pyran-6-yl)-2-(3-phenyl-1H-pyrazol-1-yl)pyrido[3,2-d]pyrimidin-4-yl)morpholine (40 mg, 0.019 mmol) in methanol (5 ml) and the resultant mixture was stirred at 20° C. for 1 h under hydrogen atmosphere. The mixture was filtered, concentrated and subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate.) to obtain 4-(2-(3-phenyl-1H-pyrazol-1-yl)-7-(tetrahydro-2H-pyran-2-yl)pyrido[3,2-d]pyrimidin-4-yl)morpholine (34.2 mg, 78%) as white solid. ¹H NMR (400 MHz, DMSO-d6) δ 8.78 (d, J=2.0 Hz, 1H), 8.72 (d, J=1.2 Hz, 1H), 8.04 (d, J=1.2 Hz, 1H), 7.99-7.97 (m, 2H), 7.50-7.38 (m, 3H), 7.08 (d, J=1.6 Hz, 1H), 4.63-4.61 (m, 5H), 4.11 (d, J=8.4 Hz, 1H), 3.83-3.52 (m, 4H), 3.36-3.29 (m, 1H), 2.01-1.90 (m, 2H), 1.60-1.49 (m, 4H); LCMS (ESI) m/z: 443.3 [M+H]⁺.

### Synthesis of 4-(2-(3-Phenyl-1H-pyrazol-1-yl)-7-(tetrahydro-2H-pyran-3-yl)pyrido[3,2-d]pyrimidin-4-yl)morpholine (Compound 19)

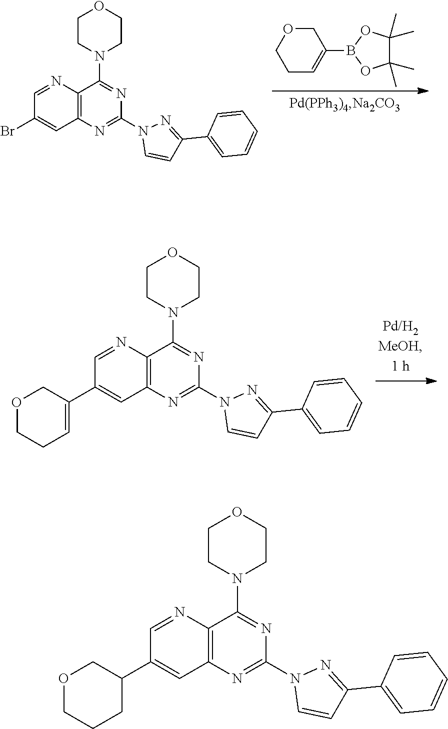 [SMILES: Brc1cnc2c(N3CCOCC3)nc(-n3ccc(-c4ccccc4)n3)nc2c1 ; CC1(C)OB(C2=CCCOC2)OC1(C)C ; C1=C(c2cnc3c(N4CCOCC4)nc(-n4ccc(-c5ccccc5)n4)nc3c2)COCC1 ; c1ccc(-c2ccn(-c3nc(N4CCOCC4)c4ncc(C5CCCOC5)cc4n3)n2)cc1]

### Step 1: Synthesis of 4-(7-(5,6-Dihydro-2H-pyran-3-yl)-2-(3-phenyl-1H-pyrazol-1-yl)pyrido[3,2-d]pyrimidin-4-yl)morpholine

A mixture of 4-(7-Bromo-2-(3-phenyl-1H-pyrazol-1-yl)pyrido[3,2-d]pyrimidin-4-yl)morpholine (60 mg, 0.14 mmol), 2-(3,4-dihydro-2H-pyran-6-yl)-4,4,5,5-tetramethyl-1,3,2-dioxaborolane (59 mg, 0.28 mmol), tetrakis(triphenylphosphine)palladium (16 mg, 0.014 mmol) and sodium carbonate (18 mg, 0.17 mmol) in water (0.5 mL) and dioxane (2.0 mL) was stirred at 80° C. for 4 under argon atmosphere. Water (25 mL) was added to the reaction mixture and then extracted with dichloromethane (25 ml*3). The combined organic layer was dried over Na₂SO₄, filtered and concentrated. The residue obtained was subjected to silica gel column chromatography (petroleum ether/ethyl acetate=4:1) to obtain 4-(7-(5,6-dihydro-2H-pyran-3-yl)-2-(3-phenyl-1H-pyrazol-1-yl)pyrido[3,2-d]pyrimidin-4-yl)morpholine (30 mg, 48%) as yellow solid. LCMS (ESI) m/z: 441.1 [M+H]⁺.

### Step 2: Synthesis of 4-(2-(3-Phenyl-1H-pyrazol-1-yl)-7-(tetrahydro-2H-pyran-3-yl)pyrido[3,2-d]pyrimidin-4-yl)morpholine

Palladium on carbon (4 mg) was added to a solution of 4-(7-(5,6-dihydro-2H-pyran-3-yl)-2-(3-phenyl-1H-pyrazol-1-yl)pyrido[3,2-d]pyrimidin-4-yl)morpholine (30 mg, 0.068 mmol) in methanol (5 ml) and the resultant mixture was stirred at 20° C. for 1 h under hydrogen atmosphere. It was then filtered and concentrated. The residue obtained was subjected to prep-HPLC(SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate.) to afford 4-(2-(3-phenyl-1H-pyrazol-1-yl)-7-(tetrahydro-2H-pyran-3-yl)pyrido[3,2-d]pyrimidin-4-yl)morpholine (6.1 mg, 20%) as white solid. ¹H NMR (400 MHz, DMSO-d6) δ 8.78 (d, J=2.4 Hz, 1H), 8.71 (d, J=2.0 Hz, 1H), 8.07 (d, J=2.0 Hz, 1H), 7.99-7.96 (m, 2H), 7.50 (t, J=4.8 Hz, 2H), 7.39 (t, J=5.4 Hz, 1H), 7.08 (d, J=2.8 Hz, 1H), 4.57 (bs, 4H), 3.97-3.94 (m, 2H), 3.91-3.81 (m, 4H), 3.56-3.47 (m, 2H), 3.08-3.06 (m, 1H), 2.07-1.72 (m, 2H), 1.71-1.69 (m, 2H); LCMS (ESI) m/z: 443.3 [M+H]⁺.

### Synthesis of 4-(7-(3,6-dihydro-2H-pyran-4-yl)-2-(3-phenyl-1H-pyrazol-1-yl)pyrido[3,2-d]pyrimidin-4-yl)morpholine (Compound 21)

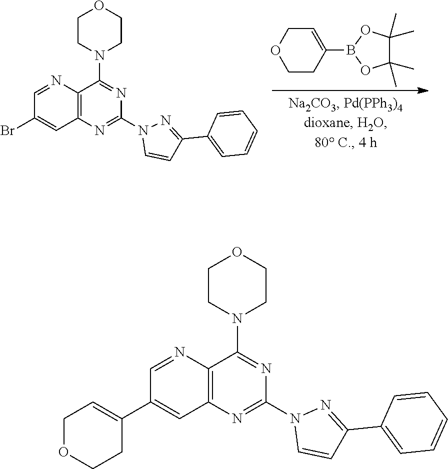 [SMILES: Brc1cnc2c(N3CCOCC3)nc(-n3ccc(-c4ccccc4)n3)nc2c1 ; CC1(C)OB(C2=CCOCC2)OC1(C)C ; C1=C(c2cnc3c(N4CCOCC4)nc(-n4ccc(-c5ccccc5)n4)nc3c2)CCOC1]

A solution of 4-(7-Bromo-2-(3-phenyl-1H-pyrazol-1-yl)pyrido[3,2-d]pyrimidin-4-yl)morpholine (60 mg, 0.14 mmol), 2-(3,6-Dihydro-2H-pyran-4-yl)-4,4,5,5-tetramethyl-1,3,2-dioxaborolane (59 mg, 0.28 mmol), tetrakis(triphenyl phosphine)palladium (16 mg, 0.014 mmol) and sodium carbonate (18 mg, 0.17 mmol) in water (0.5 mL) and dioxane (2.0 mL) was stirred at 80° C. for 4 h under argon atmosphere. Water (25 mL) was added and the resultant mixture extracted with dichloromethane (25 ml*3). The organic layer was dried over Na₂SO₄, filtered and concentrated under the reduced pressure. The residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate.) to obtain 4-(7-(3,6-Dihydro-2H-pyran-4-yl)-2-(3-phenyl-1H-pyrazol-1-yl)pyrido[3,2-d]pyrimidin-4-yl)morpholine (30 mg, 48%) as white solid. ¹H NMR (400 MHz, DMSO-d6) δ 8.96 (d, J=2.0 Hz, 1H), 8.78 (d, J=2.4 Hz, 1H), 8.08 (d, J=1.6 Hz, 1H), 7.98-7.97 (m, 3H), 7.50-7.38 (m, 3H), 7.08 (d, J=2.8 Hz, 1H), 6.78 (s, 1H), 4.59 (bs, 4H), 4.31 (d, J=2.0 Hz, 2H), 3.90-3.83 (m, 6H), 2.60 (s, 2H); LCMS (ESI) m/z: 441.3 [M+H]⁺.

### Synthesis of 4-(2-(3-phenyl-1H-pyrazol-1-yl)-7-(tetrahydrofuran-2-yl)pyrido[3,2-d]pyrimidin-4-yl)morpholine (Compound 22)

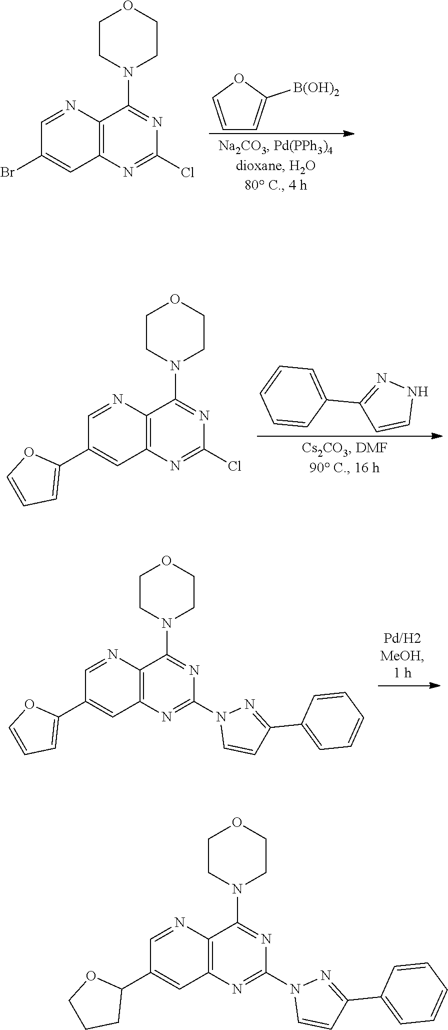 [SMILES: Clc1nc(N2CCOCC2)c2ncc(Br)cc2n1 ; OB(O)c1ccco1 ; Clc1nc(N2CCOCC2)c2ncc(-c3ccco3)cc2n1 ; c1ccc(-c2cc[nH]n2)cc1 ; c1ccc(-c2ccn(-c3nc(N4CCOCC4)c4ncc(-c5ccco5)cc4n3)n2)cc1 ; c1ccc(-c2ccn(-c3nc(N4CCOCC4)c4ncc(C5CCCO5)cc4n3)n2)cc1]

### Step 1: Synthesis of 4-(2-chloro-7-(furan-2-yl)pyrido[3,2-d]pyrimidin-4-yl)morpholine

To a solution of 4-(7-bromo-2-chloropyrido[3,2-d]pyrimidin-4-yl)morpholine (1 g, 3 mmol) in dioxane (30 mL)/H2O (6 mL) were added furan-2-ylboronic acid (376 mg, 3.35 mmol), Na2CO3 (646 mg, 6.1 mmol) and Pd(PPh3)4 (351 mg, 0.3 mmol). The resultant reaction mixture was stirred at 80° C. for 4 h under nitrogen atmosphere. It was then concentrated and the residue was subjected to silica gel chromatography (PE/EA=4:1 to 1:1) to afford 4-(2-chloro-7-(furan-2-yl)pyrido[3,2-d]pyrimidin-4-yl)morpholine (620 mg, 64%) as a yellow solid. LCMS (ESI) m/z: 317.1 [M+H]+.

### Step 2: Synthesis of 4-(7-(furan-2-yl)-2-(3-phenyl-1H-pyrazol-1-yl)pyrido[3,2-d]pyrimidin-4-yl)morpholine

To a solution of 4-(2-chloro-7-(furan-2-yl)pyrido[3,2-d]pyrimidin-4-yl)morpholine (70 mg, 0.22 mmol), 3-phenyl-1H-pyrazole (35 mg, 0.24 mmol) in DMF (5 mL) was added Cs₂CO₃ (216 mg, 0.66 mmol). The resultant reaction mixture was stirred at 90° C. for 6 h. Then the reaction was quenched with water (5 mL) and the mixture was extracted with EtOAc (20*3 mL). The organic layer was combined, washed with brine (30 mL), dried over Na2SO4, filtered and concentrated. The residue was purified by prep-HPLC (0.05% formic acid/H2O:CH3CN=5%-95%) to afford 4-(7-(furan-2-yl)-2-(3-phenyl-1H-pyrazol-1-yl)pyrido[3,2-d]pyrimidin-4-yl)morpholine (33.4 mg, 35%) as yellow solid.

¹H NMR (500 MHz, DMSO-d6) δ 9.13 (d, J=2.5 Hz, 1H), 8.80 (d, J=3.0 Hz, 1H), 8.36 (d, J=2.5 Hz, 1H), 8.00-7.99 (m, 3H), 7.52-7.46 (m, 3H), 7.40 (t, J=7.5 Hz, 1H), 7.10 (d, J=3.0 Hz, 1H), 6.76 (dd, J=3.5, 2.0 Hz, 1H), 4.57 (bs, 4H), 3.85 (t, J=4.5 Hz, 4H). LCMS (ESI) m/z: 425.3 [M+H]+.

### Step 3: Synthesis of 4-(2-(3-phenyl-1H-pyrazol-1-yl)-7-(tetrahydrofuran-2-yl)pyrido[3,2-d]pyrimidin-4-yl)morpholine

To a solution of 4-(7-(furan-2-yl)-2-(3-phenyl-1H-pyrazol-1-yl)pyrido[3,2-d]pyrimidin-4-yl)morpholine (65 mg, 0.15 mmol) in MeOH (20 mL) was added 10% Pd/C (7 mg) and the reaction mixture was stirred at room temperature for 1 h under hydrogen atmosphere. The mixture was filtered and the filtrate was concentrated. The residue was purified by Prep-HPLC (0.05% FA/H2O:CH3CN=5%-95%) to afford 4-(2-(3-phenyl-1H-pyrazol-1-yl)-7-(tetrahydrofuran-2-yl)pyrido[3,2-d]pyrimidin-4-yl)morpholine (10.6 mg, 16%) as yellow solid. ¹H NMR (400 MHz, DMSO-d6) δ 8.79 (d, J=2.8 Hz, 1H), 8.72 (d, J=2.0 Hz, 1H), 8.45 (s, 1H), 8.05 (d, J=1.6 Hz, 1H), 7.99 (d, J=6.8 Hz, 2H), 7.49 (t, J=8.4 Hz, 2H), 7.40 (t, J=8.0 Hz, 1H), 7.09 (d, J=2.4 Hz, 1H), 5.11 (t, J=7.2 Hz, 1H), 4.60 (bs, 4H), 4.13-4.06 (m, 1H), 3.92 (dd, J=14.4, 6.8 Hz, 2H), 3.85-3.83 (m, 4H), 2.47-2.43 (m, 1H), 2.01 (pent, J=7.2 Hz, 2H), 1.89-1.82 (m, 1H). LCMS (ESI) m/z: 429.1 [M+H]+.

### Synthesis of N-(3-methylphenethyl)-4-morpholinopyrido[3,2-d]pyrimidin-2-amine (Compound 23)

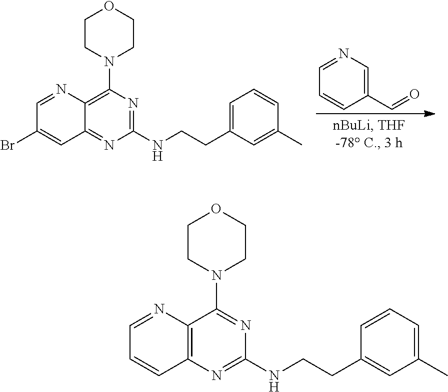 [SMILES: Cc1cccc(CCNc2nc(N3CCOCC3)c3ncc(Br)cc3n2)c1 ; O=Cc1cccnc1 ; Cc1cccc(CCNc2nc(N3CCOCC3)c3ncccc3n2)c1]

7-bromo-N-(3-methylphenethyl)-4-morpholinopyrido[3,2-d]pyrimidin-2-amine (854 mg, 2.0 mmol) was slowly added to a suspension of sodium hydride (80 mg, 2.0 mmol) in tetrahydrofuran (10 mL) at 0° C. After stirring the mixture for 10 min, it was cooled to −70° C., followed by the drop wise addition of n-butyllithium (0.8 mL 2.5 M in hexane) over a period of 15 min at −70° C. The mixture was further stirred for 20 minutes at −70° C. and nicotinaldehyde (320 mg, 3.0 mmol) in 2 mL tetrahydrofuran was added dropwise. After 2 h, the reaction was quenched with 4 mL concentrated hydrochloric acid in 5 mL water and 20 mL of diethyl ether. The organic layer was separated and washed with brine, dried over anhydrous sodium sulfate and concentrated. The resultant crude product was purified by prep-HPLC (Boston C18 21*250 mm 10 μm column. The mobile phase was acetonitrile/0.01% aqueous trifluoroacetic acid.) to afford N-(3-methylphenethyl)-4-morpholinopyrido[3,2-d]pyrimidin-2-amine (25.7 mg, 7.0%) as white solid. ¹H NMR (400 MHz, DMSO) δ 8.33 (d, J=2.5 Hz, 1H), 7.69 (s, 1H), 7.50 (dd, J=8.3, 4.0 Hz, 1H), 7.19-7.15 (m, 1H), 7.14-6.97 (m, 3H), 6.88 (s, 1H), 4.30 (bs, 4H), 3.78-3.73 (m, 4H), 3.50 (dd, J=13.2, 6.4 Hz, 2H), 2.82 (t, J=6.8 Hz, 2H), 2.28 (s, 3H); LCMS (ESI) m/z: 350.3 [M+H]⁺.

### Synthesis of (E)-4-(2-(2-(3-methylbenzylidene)hydrazinyl)pyrido[3,2-d]pyrimidin-4-yl)morpholine (Compound 24)

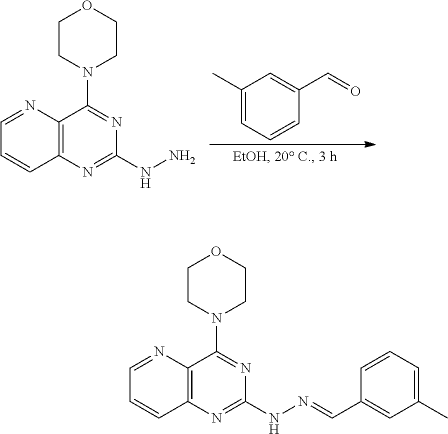

To a solution of 4-(2-hydrazinylpyrido[3,2-d]pyrimidin-4-yl)morpholine (80 mg, 0.32 mmol) and 3-methylbenzaldehyde (77 mg, 0.64 mmol) in ethanol (5.0 mL) was added acetic acid (19 mg, 0.32 mmol) and the resultant mixture was stirred at 20° C. under nitrogen for 2 h. It was concentrated and the residue was subjected to prep-HPLC (Boston C18 21*250 mm 10 μm column. The mobile phase was acetonitrile/0.01% aqueous trifluoroacetic acid.) to obtain (E)-4-(2-(2-(3-methylbenzylidene)hydrazinyl)pyrido[3,2-d]pyrimidin-4-yl)morpholine (46.6 mg, 42%) as a yellow solid. ¹H NMR (400 MHz, DMSO) δ 10.93 (s, 1H), 8.46 (dd, J=4.0, 1.6 Hz, 1H), 8.11 (s, 1H), 7.87 (d, J=8.4 Hz, 1H), 7.61 (dd, J=8.5, 4.0 Hz, 1H), 7.47 (d, J=11.2 Hz, 2H), 7.32-7.28 (m, 1H), 7.17 (d, J=7.8 Hz, 1H), 4.42 (bs, 4H), 3.79 (t, J=4.2 Hz, 4H), 2.35 (s, 3H); LCMS (ESI) m/z: 349.2 [M+H]⁺.

### Synthesis of 2-(4-morpholinopyrido[2,3-d]pyrimidin-2-yl)-5-phenyl-2,4-dihydro-3H-pyrazol-3-one (Compound 25)

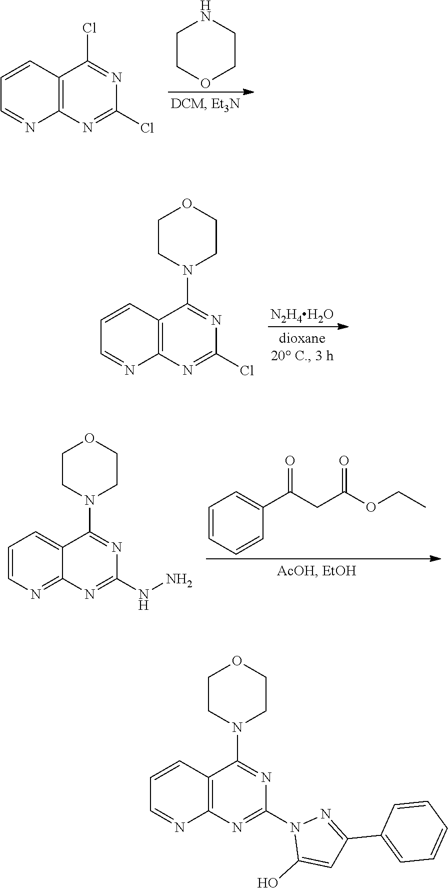

### Step 1: Synthesis of 4-(2-chloropyrido[2,3-d]pyrimidin-4-yl)morpholine

To a solution of 2,4-dichloropyrido[2,3-d]pyrimidine (0.6 g, 3 mmol) and triethylamine (600 mg, 6 mmol) in dichloromethane (10.0 mL) was added morpholine (0.27 g, 3.15 mmol) at −20° C. and the resulting solution was stirred at −20˜−10° C. under nitrogen for 30 min. The reaction was quenched with water (2 mL), dried over sodium sulfate, filtered and concentrated. The crude product was crystallized using petroleum ether and ethyl acetate (4:1) to obtain 4-(2-chloropyrido[2,3-d]pyrimidin-4-yl)morpholine (0.5 g, 45%) as off-white solid. LCMS (ESI) m/z: 251.1 [M+H]+.

### Step 2: Synthesis of 4-(2-hydrazineylpyrido[2,3-d]pyrimidin-4-yl)morpholine

To a suspension of 4-(2-chloropyrido[2,3-d]pyrimidin-4-yl)morpholine (0.25 g, 1 mmol) in 1,4-dioxane (4 mL) was added hydrazine monohydrate (0.25 g, 5 mmol), and the reaction was stirred for 0.5 h at 25° C. The mixture was filtered and concentrated to give 4-(2-hydrazineylpyrido[2,3-d]pyrimidin-4-yl)morpholine (0.18 g, 73%) as yellow solid. LCMS (ESI) m/z: 247.1 [M+H]+.

### Step 3: Synthesis of 2-(4-morpholinopyrido[2,3-d]pyrimidin-2-yl)-5-phenyl-2,4-dihydro-3H-pyrazol-3-one

A mixture of 4-(2-hydrazineylpyrido[2,3-d]pyrimidin-4-yl)morpholine (210 mg, 0.85 mmol), ethyl 3-oxo-3-phenylpropanoate (164 mg, 0.85 mmol) and acetic acid (0.1 mL) in ethanol (8 mL) was stirred at 90° C. for 2 h. The mixture was concentrated and the crude product was purified by prep-HPLC (Boston C18 21*250 mm 10 μm column. The mobile phase was acetonitrile/10 mM formic acid aqueous solution) to obtain 2-(4-morpholinopyrido[2,3-d]pyrimidin-2-yl)-5-phenyl-2,4-dihydro-3H-pyrazol-3-one as brown solid (15 mg, 5%). ¹H NMR (400 MHz, DMSO-d6) δ 8.95 (d, J=2.9 Hz, 1H), 8.55 (d, J=8.2 Hz, 1H), 7.90 (d, J=7.3 Hz, 2H), 7.52-7.43 (m, 3H), 7.39 (t, J=7.1 Hz, 1H), 6.16 (s, 1H), 4.10 (t, J=3.6 Hz, 4H), 3.84 (t, J=4.0 Hz, 4H); LCMS (ESI) m/z: 375.1 [M+H]+.

### Synthesis of 1-(4-morpholinopyrido[3,2-d]pyrimidin-2-yl)-3-phenyl-1H-pyrazol-5-ol (Compound 27)

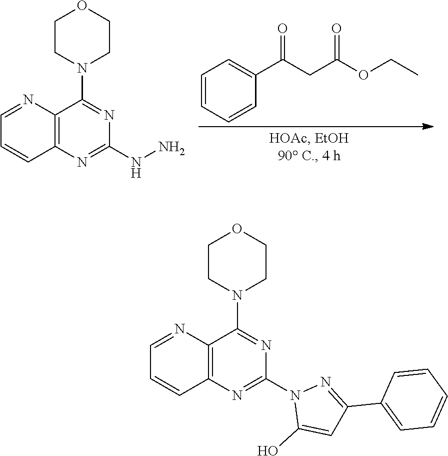 [SMILES: NNc1nc(N2CCOCC2)c2ncccc2n1 ; CCOC(=O)CC(=O)c1ccccc1 ; Oc1cc(-c2ccccc2)nn1-c1nc(N2CCOCC2)c2ncccc2n1]

A mixture of 4-(2-hydrazineylpyrido[3,2-d]pyrimidin-4-yl)morpholine (210 mg, 0.85 mmol), ethyl 3-oxo-3-phenylpropanoate (164 mg, 0.85 mmol) and acetic acid (0.1 mL) in ethanol (8 mL) was stirred at 90° C. for 4 h. The reaction mixture was concentrated and the residue was purified under prep-HPLC (Boston C18 21*250 mm 10 μm column. The mobile phase was acetonitrile/10 mM formic acid aqueous solution) to obtain 1-(4-morpholino pyrido[3,2-d]pyrimidin-2-yl)-3-phenyl-1H-pyrazol-5-ol (55.2 mg, 17%) as yellow solid. ¹H NMR (400 MHz, DMSO-d6) δ 8.76 (dd, J=4.2, 1.7 Hz, 1H), 8.29 (dd, J=8.5, 1.6 Hz, 1H), 7.92-7.87 (m, 2H), 7.84 (dd, J=8.5, 4.2 Hz, 1H), 7.46 (t, J=7.4 Hz, 2H), 7.38 (t, J=7.2 Hz, 1H), 6.17 (s, 1H), 4.66 (bs, 4H), 3.91-3.82 (m, 4H). LCMS (ESI) m/z: 375.1 [M+H]⁺.

The following compound was synthesized according to the protocol described above:

| Name | Structure | NMR, MS | # |
|---|---|---|---|
| 1-(4-morpholinopyrido[3,2- d]pyrimidin-2-yl)-3-m-tolyl- 1H-pyrazol-5-ol | 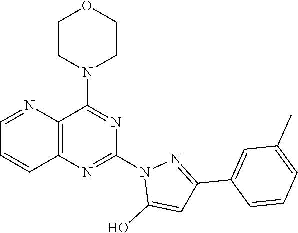 [SMILES: Cc1cccc(-c2cc(O)n(-c3nc(N4CCOCC4)c4ncccc4n3)n2)c1] | ¹H NMR (400 MHz, DMSO) δ 13.25 (bs, 1H), 8.75 (dd, J = 4.0, 1.6 Hz, 1H), 8.28 (dd, J = 8.4, 1.2 Hz, 1H), 7.84 (dd, J = 8.4 Hz, 4 Hz, 1H), 7.74-7.64 (m, 2H), 7.34 (t, J = 8.0 Hz, 1H), 7.20 (d, J = 8 Hz, 1H), 6.16 (s, 1H), 4.50 (bs, 4H), 3.85 (t, J = 4.4 Hz, 4H), 2.38 (s, 3H); LCMS (ESI) m/z: 389.1 [M + H]⁺. | 26 |

### Synthesis of 1-(3-fluorophenyl)-3-(4-morpholinopyrido[3,2-d]pyrimidin-2-yl)imidazolidin-2-one (Compound 28)

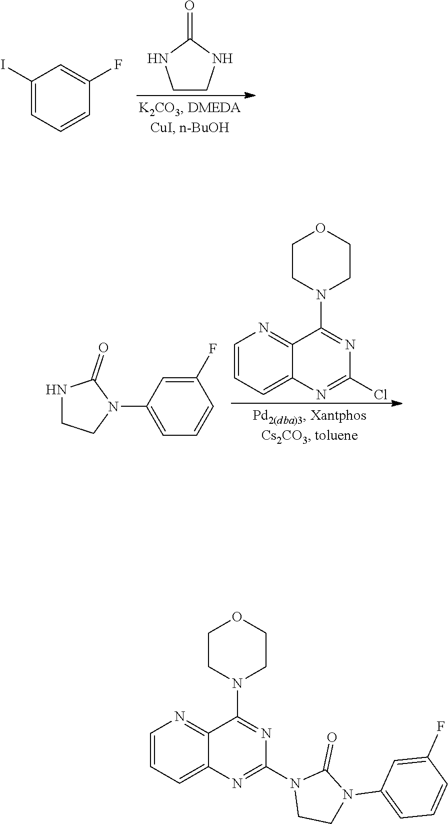

### Step 1: Synthesis of 1-(3-fluorophenyl)imidazolidin-2-one

To a solution of 1-fluoro-3-iodo-benzene (1 g, 4.50 mmol) in n-BuOH (50 mL) was added dropwise imidazolidin-2-one (1.94 g, 22.52 mmol) at 0° C. This was followed by the addition of CuI (86 mg, 451 umol), K₂CO₃ (1.87 g, 13.51 mmol) and DMEDA (119 mg, 1.35 mmol) to it and then the resultant mixture was stirred at 100° C. for 12 h. It was concentrated and the crude product was purified by flash column (ISCO 40 g silica, 60-70% ethyl acetate in petroleum ether, gradient over 20 min) to obtain 1-(3-fluorophenyl)imidazolidin-2-one (220 mg, 27%) as pale yellow gum. ¹H NMR (400 MHz, CHLOROFORM-d) Shift=7.44 (td, J=11.7, 2.2 Hz, 1H), 7.25 (s, 2H), 7.25-7.18 (m, 1H), 6.74 (ddt, J=8.2, 2.4, 1.2 Hz, 1H), 5.17 (bs, 1H), 3.91 (dd, J=8.8, 6.8 Hz, 2H), 3.67-3.50 (m, 2H)

### Step 2: Synthesis of 1-(3-fluorophenyl)-3-(4-morpholinopyrido[3,2-d]pyrimidin-2-yl)imidazolidin-2-one

To a solution of 1-(3-fluorophenyl)imidazolidin-2-one (200 mg, 1.11 mmol) and 4-(2-chloropyrido[3,2-d]pyrimidin-4-yl)morpholine (278 mg, 1.11 mmol) in toluene (5 mL) were added Cs₂CO₃ (1.08 g, 3.33 mmol), Pd₂(dba)₃ (102 mg, 111 umol) and Xantphos (64 mg, 111 umol, 0.1 eq) under nitrogen atmosphere. The mixture was stirred at 110° C. for 16 h and concentrated. The crude product was purified by prep-HPLC (Kromasil C18 (250*50 mm*10 um); column; 35-60% acetonitrile in a 10 mM ammonium bicarbonate solution in water, 10 min gradient) to obtain 1-(3-fluorophenyl)-3-(4-morpholinopyrido[3,2-d]pyrimidin-2-yl)imidazolidin-2-one (23 mg, 58 umol, 5%) as white solid. ¹H NMR (400 MHz, CHLOROFORM-d) δ=8.59-8.53 (m, 1H), 8.05-7.98 (m, 1H), 7.60-7.50 (m, 2H), 7.37-7.28 (m, 2H), 6.85-6.77 (m, 1H), 4.70 (bs, 4H), 4.31-4.21 (m, 2H), 3.99-3.86 (m, 6H). LCMS (ESI) for (C20H119FN6O2) [M+H]+: 395.1.

### Synthesis of 1-(4-morpholinopyrido[3,2-d]pyrimidin-2-yl)-3-phenyl-imidazolidin-2-one (Compound 29)

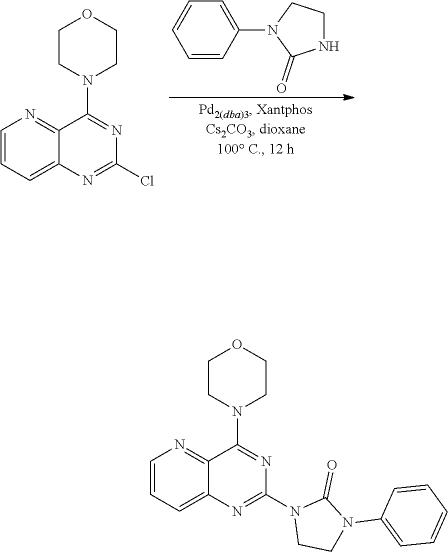 [SMILES: Clc1nc(N2CCOCC2)c2ncccc2n1 ; O=C1NCCN1c1ccccc1 ; O=C1N(c2ccccc2)CCN1c1nc(N2CCOCC2)c2ncccc2n1]

Compound 29 was synthesized according to the protocol described for the compound 28: ¹H NMR (400 MHz, CHLOROFORM-d) δ 9.08 (d, J=8.16 Hz, 1H), 8.72 (d, J=2.87 Hz, 1H), 7.75 (dd, J=8.60, 4.19 Hz, 1H), 7.58 (d, J=7.94 Hz, 2H), 7.38 (t, J=7.83 Hz, 2H), 7.20 (t, J=7.50 Hz, 1H), 5.22 (bs, 2H), 4.68 (bs, 2H), 4.40 (bs, 2H), 4.19 (bs, 2H), 3.96 (bs, 4H); LCMS: [M+H]+: 377.2

### Synthesis of 1-cyclopentyl-3-(4-morpholinopyrido[3,2-d]pyrimidin-2-yl)imidazolidin-2-one (Compound 30)

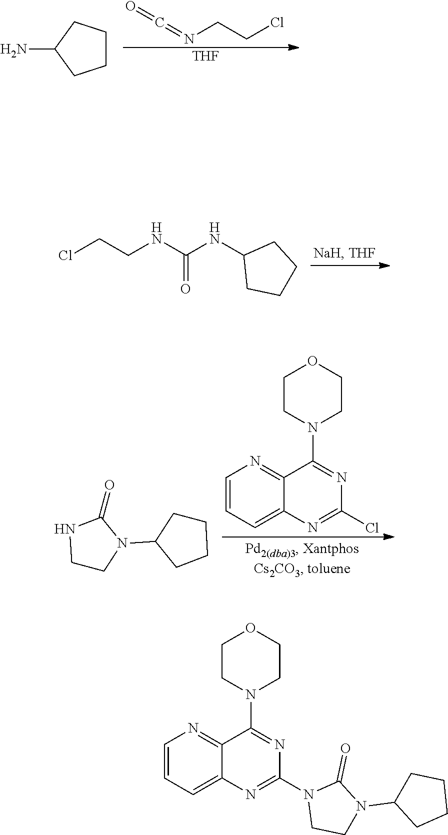

### Step 1: Synthesis of 1-(2-chloroethyl)-3-cyclopentyl-urea

To a solution of cyclopentanamine (0.6 g, 7.05 mmol) in THE (30 mL) was added dropwise 1-chloro-2-isocyanato-ethane (818 mg, 7.75 mmol) at 0° C. The mixture was stirred at 25° C. for 2 h. The resultant mixture was concentrated to obtain 1-(2-chloroethyl)-3-cyclopentyl-urea (1.3 g, 77%) as a white solid. LCMS (ESI) m/z: 191.0 [M+H]⁺

### Step 2: Synthesis of 1-cyclopentylimidazolidin-2-one

A solution of 1-(2-chloroethyl)-3-cyclopentyl-urea (0.7 g, 3.67 mmol) in THE (15 mL) was degassed and purged with nitrogen 3 times followed by the addition of NaH (367 mg, 9.18 mmol) at −20° C. The resultant mixture was stirred at −10° C. for 1 h and then at 0° C. for 1 h, and finally at 25° C. for 3 h. The mixture was then quenched with H₂O (2 mL), the aqueous phase was extracted with ethyl acetate (10 mL*3). The combined organic layers were dried over Na₂SO₄, filtered and concentrated under reduced pressure to obtain 1-cyclopentylimidazolidin-2-one (0.6 g, crude) as a pale yellow gum. LCMS (ESI) m/z: 155.3 [M+H]⁺

### Step 3: Synthesis of 1-cyclopentyl-3-(4-morpholinopyrido[3,2-d]pyrimidin-2-yl)imidazolidin-2-one

To a solution of 1-cyclopentylimidazolidin-2-one (138 mg, 896 umol) in toluene (3 mL) were added 4-(2-chloropyrido[3,2-d]pyrimidin-4-yl)morpholine (150 mg, 598 umol, Cs₂CO₃ (585 mg, 1.80 mmol), Pd₂(dba)₃ (55 mg, 60 umol) and Xantphos (35 mg, 60 umol) under nitrogen. The resultant mixture was stirred at 100° C. for 12 h. The mixture was concentrated and the residue was purified by prep-HPLC (Waters Xbridge Prep OBD C18 150*40 mm*10 um column; 25-55% acetonitrile in a 10 mM ammonium bicarbonate solution in water, 8 min gradient) to obtain 1-cyclopentyl-3-(4-morpholinopyrido[3,2-d]pyrimidin-2-yl)imidazolidin-2-one (102 mg, 274 umol, 46%) as a white solid. ¹H NMR (400 MHz, CHLOROFORM-d) δ=8.54-8.47 (m, 1H), 7.99-7.90 (m, 1H), 7.52-7.45 (m, 1H), 4.58 (bs, 4H), 4.45 (pent, J=8.4 Hz, 1H), 4.09 (t, J=8 Hz, 2H), 3.88 (t, J=4.8 Hz, 4H), 3.43 (t, J=4.4 Hz, 2H), 1.98-1.82 (m, 2H), 1.78-1.59 (m, 6H). LCMS (ESI) for C19H24N6O2 [M+H]+: 369.2.

### Synthesis of 4-ethyl-2-(4-morpholinopyrido[3,2-d]pyrimidin-2-yl)-5-phenyl-1,2,4-triazol-3-one (Compound 31)

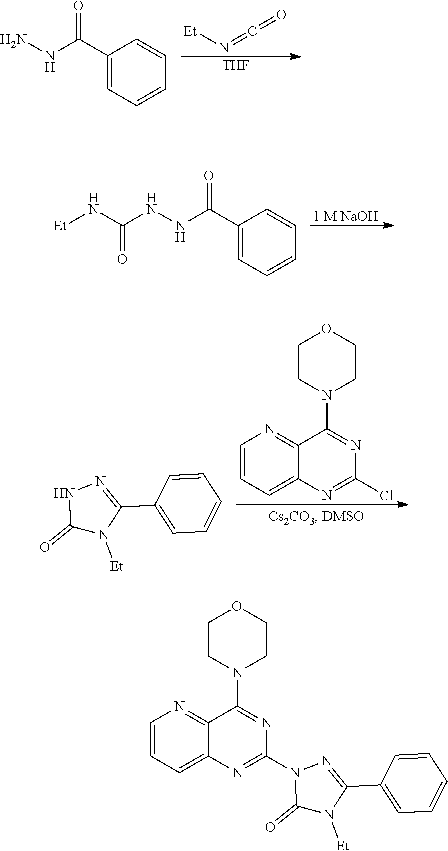

### Step 1: Synthesis of 1-benzamido-3-ethyl-urea

To a solution of benzohydrazide (1.00 g, 7.34 mmol) in THE (10 mL) was added isocyanatoethane (574 mg, 8.08 mmol) dropwise, then stirred for 14 h at 25° C. The resultant precipitate was filtered to give 1-benzamido-3-ethyl-urea (1.30 g, 85%) as white solid.

### Step 2: Synthesis of 4-ethyl-3-phenyl-1H-1,2,4-triazol-5-one

A solution of 1-benzamido-3-ethyl-urea (300 mg, 1.45 mmol) in 1 M NaOH (4 mL) was stirred for 14 h at 100° C. The resultant mixture was acidified with 1 M HCl to pH 7 and the resultant precipitate was filtered to give 4-ethyl-3-phenyl-1H-1,2,4-triazol-5-one (190 mg, 69%) as pale solid.

### Step 3: Synthesis of 4-ethyl-2-(4-morpholinopyrido[3,2-d]pyrimidin-2-yl)-5-phenyl-1,2,4-triazol-3-one

A mixture of 4-(2-chloropyrido[3,2-d]pyrimidin-4-yl)morpholine (160 mg, 0.638 mmol), 4-ethyl-3-phenyl-1H-1,2,4-triazol-5-one (181 mg, 0.957 mmol) and Cs₂CO₃ (624 mg, 1.91 mmol) in DMSO (3 mL) was stirred for 14 h at 90° C. The mixture was filtered and the filtrate was concentrated in vacuo. The crude product was purified by prep-HPLC (Waters Xbridge Prep OBD C18 150*40 mm*10 um column; 30-50% acetonitrile in 10 mM ammonium bicarbonate in water, 8 min gradient) to obtain 4-ethyl-2-(4-morpholinopyrido[3,2-d]pyrimidin-2-yl)-5-phenyl-1,2,4-triazol-3-one (60 mg, 23%) as white solid. ¹H NMR (400 MHz, CHLOROFORM-d) δ 8.64 (d, J=2.4 Hz, 1H), 8.20 (d, J=8.8 Hz, 1H), 7.70 (d, J=1.6 Hz, 2H), 7.57-7.52 (m, 4H), 4.71 (bs, 4H), 3.90-3.88 (m, 6H), 1.31 (t, J=7.6 Hz, 3H). LCMS (ESI for C21H21N7O2 [M+H]+: 404.4.

### Synthesis of 4-methyl-2-(4-morpholinopyrido[3,2-d]pyrimidin-2-yl)-5-phenyl-1,2,4-triazol-3-one (Compound 32)

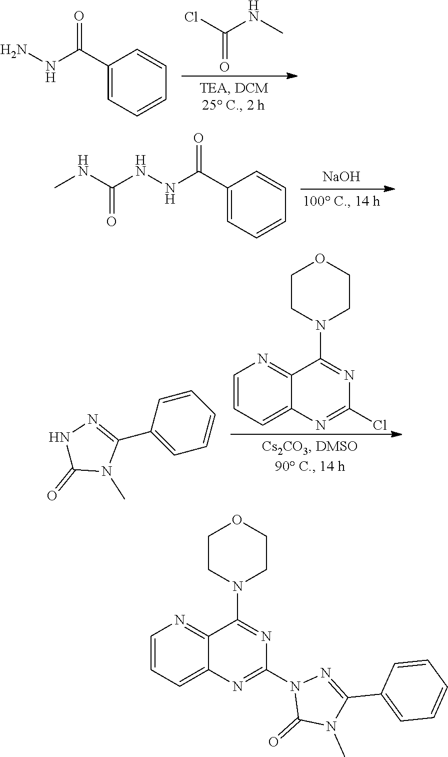

### Step 1: Synthesis of 1-benzamido-3-methyl-urea

A solution of benzohydrazide (1.00 g, 7.34 mmol), N-methylcarbamoyl chloride (2.06 g, 22.03 mmol) and TEA (2.23 g, 22.03 mmol) in DCM (10 mL) was stirred for 2 h at 15° C. The mixture was filtered, and the filtrate was concentrated in vacuo to obtain the crude product. It was purified by flash column (ISCO 20 g silica, 0-60% ethyl acetate in petroleum ether, gradient over 30 min) to obtain 1-benzamido-3-methyl-urea (540 mg) as off-white solid.

### Step 2: Synthesis of 4-methyl-3-phenyl-1H-1,2,4-triazol-5-one

A solution of 1-benzamido-3-methyl-urea (300 mg, 1.55 mmol) in 1 M NaOH (4 mL) was stirred for 14 h at 100° C. The mixture was then acidified with 1 M HCl to pH 7 and the resultant precipitate was filtered, washed with water and dried to obtain 4-methyl-3-phenyl-1H-1,2,4-triazol-5-one (130 mg, 47.79%) as off-white solid. ¹H NMR (400 MHz, DMSO-d6) δ 11.89 (bs, 1H), 7.69 (dd, J=6.42, 2.87 Hz, 2H), 7.49-7.57 (m, 3H), 3.24 (s, 3H).

### Step 3: Synthesis of 4-methyl-2-(4-morpholinopyrido[3,2-d]pyrimidin-2-yl)-5-phenyl-1,2,4-triazol-3-one

To a solution of 4-(2-chloropyrido[3,2-d]pyrimidin-4-yl)morpholine (100 mg, 400 umol) in DMSO (1 mL), were added 4-methyl-3-phenyl-1H-1,2,4-triazol-5-one (70 mg, 400 umol) and Cs₂CO₃ (391 mg, 1.20 mmol) at 90° C. for 14 h. The mixture was filtered and the filtrate was concentrated. The crude was purified by prep-HPLC (Waters Xbridge 150*40 mm*10 um, column; 25%-50% acetonitrile in an a 10 mM ammonium bicarbonate solution in water, 8 min gradient) to obtain 4-methyl-2-(4-morpholinopyrido[3,2-d]pyrimidin-2-yl)-5-phenyl-1,2,4-triazol-3-one (22 mg, 14%) as white solid. ¹H NMR (400 MHz, CHLOROFORM-d) δ 8.65 (dd, J=4.05, 1.67 Hz, 1H), 8.21 (dd, J=8.58, 1.67 Hz, 1H), 7.74 (dd, J=7.63, 1.79 Hz, 2H), 7.62-7.50 (m, 4H), 5.13-4.25 (m, 4H), 4.01-3.89 (m, 4H), 3.45 (s, 3H), LCMS (ESI) for C20H19N7O2 [M+H]+: 390.2.

### Synthesis of tert-butyl 4-[4-(4-morpholinopyrido[3,2-d]pyrimidin-2-yl)pyrimidin-2-yl]piperidine-1-carboxylate (Compound 33), 4-[2-[2-(4-piperidyl)pyrimidin-4-yl]pyrido[3,2-d]pyrimidin-4-yl]morpholine (Compound 37) and 4-[2-[2-(1-methyl-4-piperidyl)pyrimidin-4-yl]pyrido[3,2-d]pyrimidin-4-yl]morpholine (Compound 38)

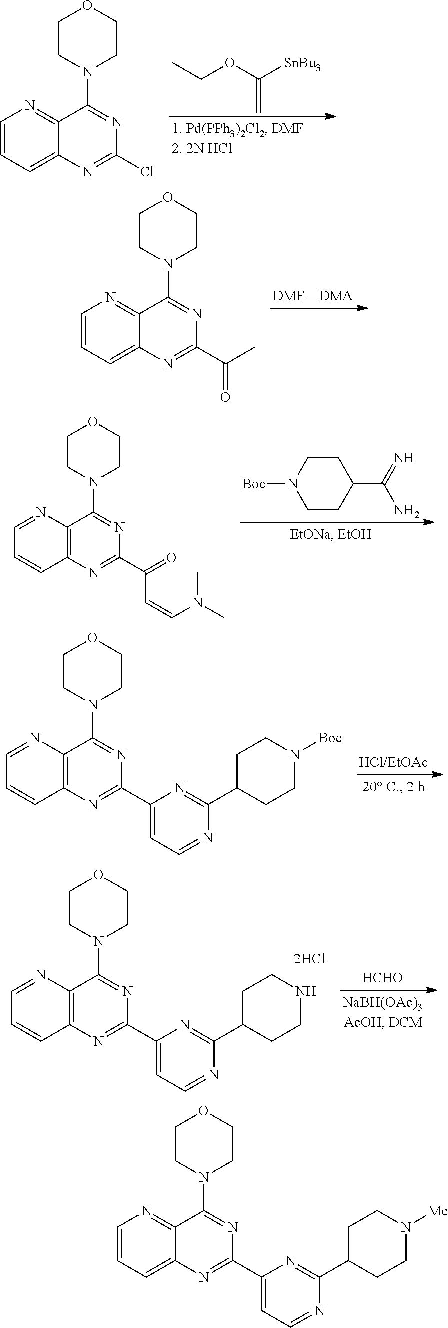

### Step 1: Synthesis of 1-(4-morpholinopyrido[3,2-d]pyrimidin-2-yl)ethanone

To a solution of 4-(2-chloropyrido[3,2-d]pyrimidin-4-yl)morpholine (5 g, 19.95 mmol) in DMF (100 mL) were added tributyl(1-ethoxyvinyl)stannane (8.64 g, 23.93 mmol) and Pd(dppf)Cl₂ (146 mg, 200 umol) under nitrogen atmosphere and the resultant mixture was stirred at 100° C. for 48 h. The mixture was cooled and then ethyl acetate (5 mL) and KF (4 g in 50 mL of water) were added and the resultant mixture was stirred at 25° C. for 3 h. The layers were separated, the aqueous phase was extracted with acetate (10 mL*3), the combined organic layers were washed with saturated NaHCO₃ (10 mL), brine (10 mL), dried over Na₂SO₄ and concentrated. The resultant crude product was dissolved in THE (2 mL), and HCl (2 M, 2 mL) was added and the mixture was stirred at 40° C. for 3 h. Water (30 mL) was added to the mixture and the aqueous solution was extracted with ethyl acetate (30 mL*8), The combined organic phase was washed with brine (20 mL*3), dried with anhydrous Na₂SO₄, filtered and concentrated. The crude product was purified by flash column (ISCO 40 g silica, 0-80% ethyl acetate in petroleum ether, gradient over 20 min) to obtain 1-(4-morpholinopyrido[3,2-d]pyrimidin-2-yl)ethanone (2.3 g, 8.91 mmol, 44.65%) as yellow solid; LCMS (ESI) m/z: 259.1 [M+H]⁺. ¹H NMR (400 MHz, CHLOROFORM-d) δ 8.78 (dd, J=4.1, 1.6 Hz, 1H), 8.29 (dd, J=8.5, 1.6 Hz, 1H), 7.67 (dd, J=8.5, 4.1 Hz, 1H), 4.52 (bs, 4H), 3.90 (t, J=4.8 Hz, 4H), 2.76 (s, 3H).

### Step 2: Synthesis of (Z)-3-(dimethylamino)-1-(4-morpholinopyrido[3,2-d]pyrimidin-2-yl)prop-2-en-1-one

A mixture of 1-(4-morpholinopyrido[3,2-d]pyrimidin-2-yl)ethanone (2.1 g, 8.13 mmol) in DMF-DMA (8.97 g, 75.28 mmol) was stirred at 100° C. for 16 h. It was cooled and the resultant precipitate was filtered, the solid was collected, washed with ethyl acetate (15 mL*3) and dried to obtain (Z)-3-(dimethylamino)-1-(4-morpholinopyrido[3,2-d]pyrimidin-2-yl)prop-2-en-1-one (1.7 g, 67%) as yellow solid. LCMS (ESI) m/z: 314.2 [M+H]⁺.

### Step 3: Synthesis of tert-butyl 4-[4-(4-morpholinopyrido[3,2-d]pyrimidin-2-yl)pyrimidin-2-yl]piperidine-1-carboxylate

To a mixture of tert-butyl 4-carbamimidoylpiperidine-1-carboxylate (130 mg, 574.43 umol) in EtOH (4 mL), were added (Z)-3-(dimethylamino)-1-(4-morpholinopyrido[3,2-d]pyrimidin-2-yl)prop-2-en-1-one (120 mg, 383 umol) and EtONa (52 mg, 766 umol). The resultant mixture was stirred at 80° C. for 16 h, then cooled, filtered and the filtrate was concentrated. The crude product was purified by prep-HPLC (Waters Xbridge Prep OBD C18 150*40 mm*10 um column; 30%-70% acetonitrile in an a 0.05% ammonium hydroxide and 10 mM ammonium bicarbonate solution, 8 min gradient) to obtain tert-butyl 4-[4-(4-morpholinopyrido[3,2-d]pyrimidin-2-yl)pyrimidin-2-yl]piperidine-1-carboxylate (50 mg, 27%) as pale yellow solid. ¹H NMR (400 MHz, CHLOROFORM-d) δ=8.87 (d, J=5.1 Hz, 1H), 8.76 (dd, J=4.2, 1.8 Hz, 1H), 8.37 (dd, J=8.5, 1.7 Hz, 1H), 8.19 (d, J=5.1 Hz, 1H), 7.67 (dd, J=8.6, 4.2 Hz, 1H), 4.64 (bs, 4H), 4.26 (bs, 2H), 3.94 (t, J=4.8 Hz, 4H), 3.30 (tt, J=11.7, 3.7 Hz, 1H), 2.90-2.80 (m, 2H), 2.13-2.03 (m, 2H), 1.94 (dq, J=8.4, 4.4 Hz, 2H), 1.48 (s, 9H). LCMS (ESI) for C25H31N7O3 [M+H]+: 478.2.

### Step 4: Synthesis of 4-[2-[2-(4-piperidyl)pyrimidin-4-yl]pyrido[3,2-d]pyrimidin-4-yl]morpholine

To a mixture of tert-butyl 4-[4-(4-morpholinopyrido[3,2-d]pyrimidin-2-yl)pyrimidin-2-yl]piperidine-1-carboxylate (380 mg, 796 umol) in ethyl acetate (8 mL), was added HCl/EtOAc (20 mL) and the resulting mixture was stirred at 20° C. for 2 h. It was concentrated and the crude product was purified by prep-HPLC (Welch Xtimate C18 150*25 mm*5 um column; 10%-30% acetonitrile in a 0.04% hydrochloric acid solution in water, 10 min gradient) to obtain 4-[2-[2-(4-piperidyl)pyrimidin-4-yl]pyrido[3,2-d]pyrimidin-4-yl]morpholine (42 mg, 13%) as orange solid. ¹HNMR (400 MHz, METHANOL-d4) δ=9.17 (d, J=5.1 Hz, 1H), 9.02 (dd, J=4.1, 1.3 Hz, 1H), 8.75 (dd, J=8.6, 1.3 Hz, 1H), 8.49 (d, J=5.0 Hz, 1H), 8.06 (dd, J=8.6, 4.3 Hz, 1H), 5.32 (bs, 2H), 4.62 (bs, 2H), 4.01 (t, J=4.8 Hz, 4H), 3.63-3.47 (m, 3H), 3.29-3.22 (m, 2H), 2.49-2.40 (m, 2H), 2.39-2.25 (m, 2H). LCMS (ESI) for C20H25C12N7O [M+H]+: 378.2

### Step 5: Synthesis of 4-[2-[2-(1-methyl-4-piperidyl)pyrimidin-4-yl]pyrido[3,2-d]pyrimidin-4-yl]morpholine

To a solution of 4-[2-[2-(4-piperidyl)pyrimidin-4-yl]pyrido[3,2-d]pyrimidin-4-yl]morpholine (200 mg, 530 umol) in DCM (10 mL), were added formaldehyde (129. mg, 1.59 mmol) and NaBH(OAc)₃ (225 mg, 1.06 mmol) and the resulting mixture was stirred at 20° C. for 16 h. The mixture was then filtered, and the filtrate was purified by prep-HPLC (Phenomenex Gemini-NX 150*30 mm*5 um column; 5%-35% acetonitrile in an a 10 mM ammonium bicarbonate solution, 8 min gradient) to obtain 4-[2-[2-(1-methyl-4-piperidyl)pyrimidin-4-yl]pyrido[3,2-d]pyrimidin-4-yl]morpholine (64 mg, 30%) as pale yellow solid. ¹H NMR (400 MHz, CHLOROFORM-d) δ=8.87 (bs, 1H), 8.75 (bs, 1H), 8.35 (d, J=8.8 Hz, 1H), 8.17 (bs, 1H), 7.70-7.61 (m, 1H), 4.63 (bs, 4H), 3.94 (bs, 4H), 3.11 (bs, 1H), 3.00 (bs, 2H), 2.33 (bs, 3H), 2.12 (bs, 6H). LCMS (ESI) for C21H25N7O [M+H]+: 392.2.

### Synthesis of tert-butyl 4-[4-(4-morpholinopyrido[3,2-d]pyrimidin-2-yl)pyrimidin-2-yl]piperazine-1-carboxylate (Compound 55) and 4-[2-(2-piperazin-1-ylpyrimidin-4-yl)pyrido[3,2-d]pyrimidin-4-yl]morpholine (Compound 34)

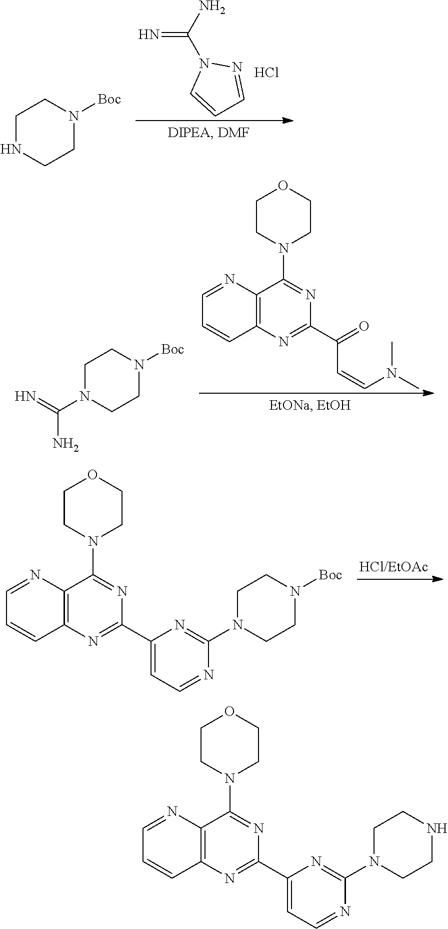

### Step 1: Synthesis of tert-butyl 4-carbamimidoylpiperazine-1-carboxylate

To a solution of tert-butyl piperazine-1-carboxylate (500 mg, 2.68 mmol) and pyrazole-1-carboxamidine;hydrochloride (394 mg, 2.68 mmol) in DMF (10 mL) was added DIPEA (35 mg, 269 umol) and the mixture was stirred at 20° C. for 16 h. It was concentrated under reduced pressure to give a crude product tert-butyl 4-carbamimidoylpiperazine-1-carboxylate (1.2 g, crude) as white solid; LCMS (ESI) m/z: 229.1 [M+H]⁺

### Step 2: Synthesis of tert-butyl 4-[4-(4-morpholinopyrido[3,2-d]pyrimidin-2-yl)pyrimidin-2-yl]piperazine-1-carboxylate

To a mixture of tert-butyl 4-carbamimidoylpiperazine-1-carboxylate (328 mg, 1.44 mmol) in EtOH (8 mL) was added (Z)-3-(dimethylamino)-1-(4-morpholinopyrido[3,2-d]pyrimidin-2-yl)prop-2-en-1-one (300 mg, 958 umol) and EtONa (130 mg, 1.91 mmol) and the resultant mixture was stirred at 80° C. for 16 h. The mixture was filtered and the filtrated was concentrated to give 700 mg crude product which was purified by prep-HPLC (Waters Xbridge Prep OBD C18 150*40 mm*10 um column; 40%-70% acetonitrile in an a 10 mM ammonium bicarbonate solution, 8 min gradient) to obtain tert-butyl 4-[4-(4-morpholinopyrido[3,2-d]pyrimidin-2-yl)pyrimidin-2-yl]piperazine-1-carboxylate (275 mg) as pale yellow solid. ¹H NMR (400 MHz, CHLOROFORM-d) δ=8.74 (dd, J=4.1, 1.7 Hz, 1H), 8.51 (d, J=5.1 Hz, 1H), 8.28 (dd, J=1.5, 8.4 Hz, 1H), 7.65 (dd, J=8.5, 4.1 Hz, 1H), 7.59 (d, J=4.9 Hz, 1H), 4.62 (bs, 4H), 4.00-3.90 (m, 8H), 3.61-3.51 (m, 4H), 1.50 (s, 9H). LCMS (ESI) for C24H30N8O3 [M+H]+: 479.3.

### Step 3: Synthesis of 4-[2-(2-piperazin-1-ylpyrimidin-4-yl)pyrido[3,2-d]pyrimidin-4-yl]morpholine

To a solution of tert-butyl 4-[4-(4-morpholinopyrido[3,2-d]pyrimidin-2-yl)pyrimidin-2-yl]piperazine-1-carboxylate (500 mg, 1.04 mmol) in EtOAc (5 mL) was added HCl/EtOAc (15 mL) and then the resultant mixture was stirred at 20° C. for 1 h. It was then filtered, and the filtrate was purified by prep-HPLC (Phenomenex Luna C8 250*50 mm*10 um column; 1%-20% acetonitrile in a 0.05% hydrochloric acid solution in water, 10 min gradient) to obtain 4-[2-(2-piperazin-1-ylpyrimidin-4-yl)pyrido[3,2-d]pyrimidin-4-yl]morpholine·HCl (282 mg, 65%) as a yellow solid. 1H NMR (400 MHz, METHANOL-d4) δ=9.01 (d, J=4.2 Hz, 1H), 8.80-8.75 (m, 2H), 8.04 (dd, J=8.6, 4.2 Hz, 1H), 7.85 (d, J=4.9 Hz, 1H), 5.33-5.30 (m, 2H), 4.58 (bs, 2H), 4.38-4.36 (m, 4H), 4.01-3.99 (m, 4H), 3.40 (br d, J=4.4 Hz, 4H). LCMS (ESI) for C19H22N8O [M+H]+: 379.2.

The following compounds were synthesized according to the protocol described above.

| Name | Structure | NMR, MS | # |
|---|---|---|---|
| 4-[2-(2- tetrahydropyran-4- ylpyrimidin-4- yl)pyrido[3,2- d]pyrimidin-4- yl]morpholine | 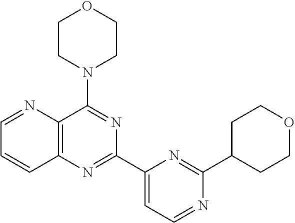 [SMILES: c1cnc2c(N3CCOCC3)nc(-c3ccnc(C4CCOCC4)n3)nc2c1] | ¹H NMR (400 MHz, CHLOROFORM-d) δ = 8.89 (d, J = 5.1 Hz, 1H), 8.76 (dd, J = 4.2, 1.8 Hz, 1H), 8.36 (dd, J = 8.4, 1.8 Hz, 1H), 8.20 (d, J = 5.1 Hz, 1H), 7.67 (dd, J = 8.4, 4.2 Hz, 1H), 4.64 (bs, 4H), 4.17 − 4.07 (m, 2H), 3.98 − 3.90 (m, 4H), 3.59 (dt, J = 11.6, 2.3 Hz, 2H), 3.40 (tt, J = 11.4, 4.0 Hz, 1H), 2.22 − 1.99 (m, 4H). LCMS (ESI) for C20H22N6O2 [M + H]+:379.2. | 35 |
| 4-[4-(4- morpholinopyrido[3,2- d]pyrimidin-2- yl)pyrimidin-2- yl]morpholine | 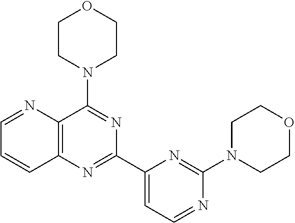 [SMILES: c1cnc2c(N3CCOCC3)nc(-c3ccnc(N4CCOCC4)n3)nc2c1] | ¹H NMR (400 MHz, CHLOROFORM-d) δ 8.74 (dd, J = 4.1, 1.7 Hz, 1H), 8.52 (d, J = 4.9 Hz, 1H), 8.28 (dd, J = 8.4, 1.8 Hz, 1H), 7.65 (dd, J = 8.4, 4.2 Hz, 1H), 7.61 (d, J = 5.1 Hz, 1H), 4.62 (bs, 4H), 4.0-3.85 (m, 8H), 3.85 − 3.80 (m, 4H). LCMS (ESI) for C19H21N7O2 [M + H]+: 380.2. | 51 |
| 4-(2-(2- phenylpyrimidin-4- yl)pyrido[3,2- d]pyrimidin-4- yl)morpholine | 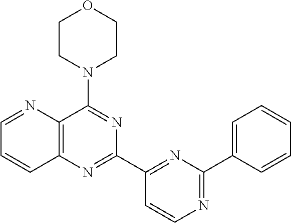 [SMILES: c1ccc(-c2nccc(-c3nc(N4CCOCC4)c4ncccc4n3)n2)cc1] | ¹H NMR (400 MHz, DMSO-d₆) δ 9.10 (d, J = 5.1 Hz, 1H), 8.89 (dd, J = 4.1, 1.7 Hz, 1H), 8.54 (dd, J = 7.3, 2.4 Hz, 2H), 8.37 (dd, J = 8.5, 1.7 Hz, 1H), 8.33 (d, J = 5.1 Hz, 1H), 7.90 (dd, J = 8.5, 4.1 Hz, 1H), 7.60-7.57 (m, 3H), 4.61 (bs, 4H), 3.85 (t, J = 4 Hz, 4H). LCMS (ESI) m/z: 371.3 [M + H]+. | 39 |

### Synthesis of tert-butyl 4-[6-(4-morpholinopyrido[3,2-d]pyrimidin-2-yl)-2-pyridyl]piperidine-1-carboxylate (Compound 56) and 4-[2-[6-(4-piperidyl)-2-pyridyl]pyrido[3,2-d]pyrimidin-4-yl]morpholine (Compound 36)

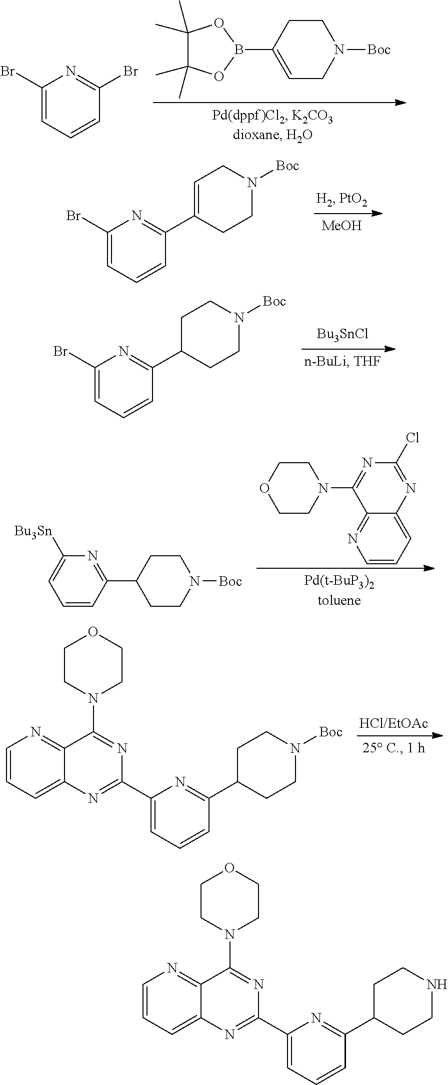

### Step 1: Synthesis of tert-butyl 4-(6-bromo-2-pyridyl)-3,6-dihydro-2H-pyridine-1-carboxylate

To a solution of 2,6-dibromopyridine (2 g, 8.44 mmol) and tert-butyl 4-(4,4,5,5-tetramethyl-1,3,2-dioxaborolan-2-yl)-3,6-dihydro-2H-pyridine-1-carboxylate (3.13 g, 10.13 mmol) in dioxane (20 mL) and H₂O (8 mL) were added Pd(dppf)Cl₂·CH2Cl₂ (689 mg, 844 umol) and K₂CO₃ (2M in water, 8.44 mL). The resultant mixture was stirred at 90° C. under nitrogen for 12 h. Water (10 mL) was added to the mixture and it was extracted with EtOAc (20 mL*4). The organic layer was washed with brine (10 mL), dried over Na₂SO₄ and concentrated to give the crude product. It was purified by flash column (ISCO 40 g silica, 20-40% ethyl acetate in petroleum ether, over 20 min) to obtain tert-butyl 4-(6-bromo-2-pyridyl)-3,6-dihydro-2H-pyridine-1-carboxylate (1.5 g, 52%, Product-A) and tert-butyl 4-[6-(1-tert-butoxycarbonyl-3,6-dihydro-2H-pyridin-4-yl)-2-pyridyl]-3,6-dihydro-2H-pyridine-1-carboxylate (1.2 g, 32%, Product-B) as white solids. Product-A: ¹H NMR (400 MHz, CHLOROFORM-d) δ=7.42 (t, J=7.6 Hz, 1H), 7.30-7.15 (m, 2H), 6.62 (bs, 1H), 4.09-4.03 (m, 2H), 3.56 (bs, 2H), 2.52 (bs, 2H), 1.42 (s, 9H).

### Step 2: Synthesis of methyl tert-butyl 4-(6-bromo-2-pyridyl)piperidine-1-carboxylate

To a solution of tert-butyl 4-(6-bromo-2-pyridyl)-3,6-dihydro-2H-pyridine-1-carboxylate (1.5 g, 4.42 mmol) in EtOAc (20 mL) was added PtO₂ (100 mg, 442 umol) under nitrogen atmosphere. The suspension was degassed under vacuum and purged with hydrogen several times. The mixture was stirred under hydrogen (15 psi) at 15° C. for 6 h. The reaction mixture was filtered and the filtrate was concentrated. The crude product was purified by flash column (ISCO 20 g silica, 10-30% ethyl acetate in petroleum ether, gradient over 20 min) to obtain tert-butyl 4-(6-bromo-2-pyridyl)piperidine-1-carboxylate (0.8 g, 53%) as white solid.

### Step 3: Synthesis of tert-butyl 4-(6-tributylstannyl-2-pyridyl)piperidine-1-carboxylate

To a solution of tert-butyl 4-(6-bromo-2-pyridyl)piperidine-1-carboxylate (0.5 g, 1.47 mmol) in THE (10 mL) was added n-BuLi (2.5M, 762 uL) at −70° C. and the mixture was stirred at −70° C. for 1 h. Then tributyl(chloro)stannane (572 mg, 1.76 mmol) was added to the above solution at −70° C. and stirred for 1 h at that temperature and then at 20° C. for 12 h. 15 mL of water was added to the mixture and it was extracted with ethyl acetate (30 mL*2). The combined organic layers were washed with brine (15 mL) and dried over Na₂SO₄. Concentration and purification of the crude product by flash column (ISCO 10 g silica, 0-10% ethyl acetate in petroleum ether, gradient over 20 min) yielded tert-butyl 4-(6-tributylstannyl-2-pyridyl)piperidine-1-carboxylate (80 mg, 10%) as a colorless oil.

### Step 4: Synthesis of tert-butyl 4-[6-(4-morpholinopyrido[3,2-d]pyrimidin-2-yl)-2-pyridyl]piperidine-1-carboxylate

To a solution of tert-butyl 4-(6-tributylstannyl-2-pyridyl)piperidine-1-carboxylate (80 mg, 145 umol) in toluene (4 mL) were added 4-(2-chloropyrido[3,2-d]pyrimidin-4-yl)morpholine (36 mg, 145 umol) and Pd(t-Bu₃P)₂ (7 mg, 15 umol). Then the mixture was stirred at 100° C. for 12 h and concentrated. The crude product obtained was purified by prep-HPLC (Waters Xbridge Prep OBD C18 150*40 10u column; 40-70% acetonitrile in an a 10 mM ammonium bicarbonate solution in water, 8 min gradient) to obtain tert-butyl 4-[6-(4-morpholinopyrido[3,2-d]pyrimidin-2-yl)-2-pyridyl]piperidine-1-carboxylate (12 mg, 17%) as pale yellow solid. ¹H NMR (400 MHz, CHLOROFORM-d) δ=8.75-8.67 (m, 1H), 8.37 (dd, J=8, 5, 1.4 Hz, 1H), 8.32 (d, J=7.6 Hz, 1H), 7.81 (t, J=7.8 Hz, 1H), 7.64 (dd, J=8.5, 4.1 Hz, 1H), 7.30-7.28 (m, 1H), 4.63 (bs, 4H), 4.28 (bs, 2H), 4.04-3.90 (m, 4H), 3.28-3.11 (m, 1H), 2.88 (bt, J=12.6 Hz, 2H), 2.08 (br d, J=12.4 Hz, 2H), 1.82-1.66 (m, 2H), 1.50 (s, 9H). LCMS (ESI) for C26H32N6O3 [M+H]+: 477.3.

### Step 5: Synthesis of 4-[2-[6-(4-piperidyl)-2-pyridyl]pyrido[3,2-d]pyrimidin-4-yl]morpholine

A solution of tert-butyl 4-[6-(4-morpholinopyrido[3,2-d]pyrimidin-2-yl)-2-pyridyl]piperidine-1-carboxylate (9 mg, 19 umol) in 4M HCl/EtOAc (5 mL) was stirred at 25° C. for 30 min. The reaction mixture was concentrated and 10 mL deionized water was added to the residue. The resultant mixture was lyophilized to obtain 4-[2-[6-(4-piperidyl)-2-pyridyl]pyrido[3,2-d]pyrimidin-4-yl]morpholine·3HCl (8 mg, 89%) as pale yellow solid. ¹H NMR (400 MHz, METHANOL-d4) δ=8.98 (dd, J=4.2, 1.4 Hz, 1H), 8.84-8.71 (m, 1H), 8.57 (d, J=7.6 Hz, 1H), 8.13 (t, J=7.8 Hz, 1H), 8.03 (dd, J=8.6, 4.3 Hz, 1H), 7.72 (d, J=7.8 Hz, 1H), 5.31 (bs, 2H), 4.60 (bs, 2H), 4.01 (bs, 4H), 3.60 (bd, J=12.8 Hz, 2H), 3.39-3.33 (m, 1H), 3.28-3.19 (m, 2H), 2.38-2.23 (m, 4H). LCMS (ESI) for C21H24N6O [M+H]+: 377.2.

### Synthesis of 4-(2-(3-(3-fluorophenyl)-1H-pyrazol-1-yl)pyrido[3,2-d]pyrimidin-4-yl)morpholine (Compound 41) and 4-(2-(5-(3-fluorophenyl)-1H-pyrazol-1-yl)pyrido[3,2-d]pyrimidin-4-yl)morpholine (Compound 40)

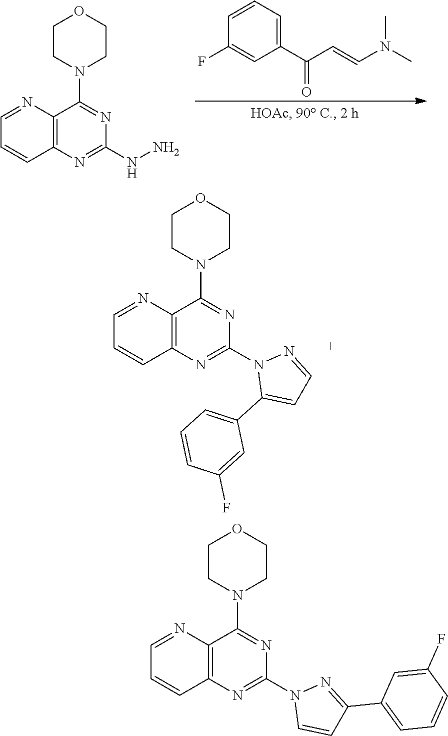

A mixture of 4-(2-hydrazineylpyrido[3,2-d]pyrimidin-4-yl)morpholine (130 mg, 0.0.5 mmol) and (E)-3-(dimethylamino)-1-(3-fluorophenyl)prop-2-en-1-one (184 mg, 0.95 mmol) in acetic acid (3 mL) was stirred at 90° C. for 2 h. The reaction mixture was concentrated and the residue was purified by prep-HPLC (SunFire C18, 4.6*50 mm, 3.5 um column. The mobile phase was acetonitrile/10 mM ammonium bicarbonate aqueous solution.) to obtain the target compounds:

Compound 41: 4-(2-(3-(3-fluorophenyl)-1H-pyrazol-1-yl)pyrido[3,2-d]pyrimidin-4-yl)morpholine (2.6 mg, 1%) was isolated as white solid. ¹H NMR (400 MHz, DMSO-d6) δ 8.82 (d, J=2.7 Hz, 1H), 8.76 (dd, J=4.1, 1.7 Hz, 1H), 8.22 (dd, J=8.5, 1.7 Hz, 1H), 7.84 (dd, J=8.5, 4.3 Hz, 2H), 7.78 (d, J=9.7 Hz, 1H), 7.54 (dd, J=14.2, 8.0 Hz, 1H), 7.24 (t, J=8.4 Hz, 1H), 7.16 (d, J=2.7 Hz, 1H), 4.58 (bs, 4H), 3.87-3.81 (m, 4H). LCMS (ESI) m/z: 377.1 [M+H]⁺.

Compound 40: 4-(2-(5-(3-fluorophenyl)-1H-pyrazol-1-yl)pyrido[3,2-d]pyrimidin-4-yl)morpholine (12 mg, 6%) was isolated as white solid. ¹H NMR (400 MHz, DMSO-d6) δ 8.78 (dd, J=4.1, 1.7 Hz, 1H), 8.11 (dd, J=8.5, 1.7 Hz, 1H), 7.86-7.78 (m, 2H), 7.41 (dd, J=15.3, 7.6 Hz, 1H), 7.21 (dd, J=7.1, 4.3 Hz, 2H), 7.11 (d, J=7.8 Hz, 1H), 6.70 (d, J=1.6 Hz, 1H), 3.58 (bs, 4H), 3.53 (s, 4H). LCMS (ESI) m/z: 377.1 [M+H]⁺.

### Synthesis of 4-(2-(3-(tetrahydro-2H-pyran-4-yl)-1H-pyrazol-1-yl)pyrido[3,2-d]pyrimidin-4-yl)morpholine (Compound 45)

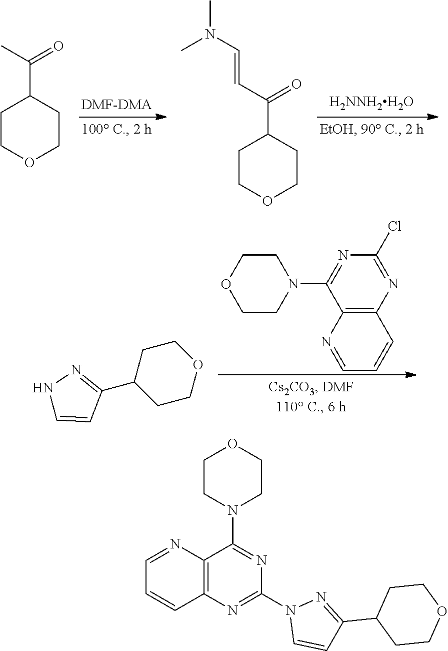

### Step 1: Synthesis of 3-(dimethylamino)-1-(tetrahydro-2H-pyran-4-yl)prop-2-en-1-one

To a solution of 1-(tetrahydro-2H-pyran-4-yl)ethan-1-one (1 g, 7.81 mmol) in toluene (10 ml) was added N,N-dimethylformamide dimethylacetal (2.79 g, 23.4 mmol). Then the reaction mixture was stirred at 100° C. for 2 h and concentrated to afford 3-(dimethylamino)-1-(tetrahydro-2H-pyran-4-yl)prop-2-en-1-one (1.2 g, 62%). LCMS (ESI) m/z: 184.2 [M+H]⁺.

### Step 2: Synthesis of 3-(tetrahydro-2H-pyran-4-yl)-1H-pyrazole

To a solution of 3-(dimethylamino)-1-(tetrahydro-2H-pyran-4-yl)prop-2-en-1-one (1.1 g, 6 mmol) in ethanol (10 mL) was added hydrazine hydrate (5 mL) and the mixture was stirred at 90° C. for 2 h. It was then concentrated and the residue was purified by prep-HPLC (Boston C18 21*250 mm 10 μm column. The mobile phase was acetonitrile/0.05% trifluoroacetic acid aqueous solution) to obtain 3-(tetrahydro-2H-pyran-4-yl)-1H-pyrazole (0.67 g, 64%) as yellow oil. LCMS (ESI) m/z: 153.3 [M+H]⁺.

### Step 3: Synthesis of 4-(2-(3-(tetrahydro-2H-pyran-4-yl)-1H-pyrazol-1-yl)pyrido[3,2-d]pyrimidin-4-yl)morpholine

To a solution of 4-(2-chloropyrido[3,2-d]pyrimidin-4-yl)morpholine (200 mg, 0.8 mmol) and 3-(tetrahydro-2H-pyran-4-yl)-1H-pyrazole (134 mg, 0.88 mmol) in N,N-dimethylformamide (10 mL) was added cesium carbonate (782 mg, 2.4 mmol). The reaction mixture was stirred at 110° C. for 6 h and concentrated. The residue obtained subjected to prep-HPLC (Boston C18 21*250 mm 10 μm column. The mobile phase was acetonitrile/10 mM formic acid aqueous solution) to obtain 4-(2-(3-(tetrahydro-2H-pyran-4-yl)-1H-pyrazol-1-yl)pyrido[3,2-d]pyrimidin-4-yl)morpholine (101.5 mg, 34.7%) as white solid. ¹H NMR (400 MHz, DMSO-d) δ 8.73 (dd, J=4.0, 1.6 Hz, 1H), 8.62 (d, J=2.8 Hz, 1H), 8.15 (dd, J=8.4, 1.6 Hz, 1H), 7.80 (dd, J=8.8, 4.0 Hz, 1H), 6.47 (d, J=2.8 Hz, 1H), 4.54 (bs, 4H), 3.95-3.93 (m, 2H), 3.82 (t, J=4.8, 4H), 3.50-3.46 (m, 2H), 3.00-2.94 (m, 1H), 1.87-1.84 (m, 2H), 1.77-1.68 (m, 2H). LCMS (ESI) m/z: 367.0 [M+H]⁺.

### Synthesis of 4-(2-(3-phenyl-1H-pyrazol-1-yl)pyrido[4,3-d]pyrimidin-4-yl)morpholine (Compound 46)

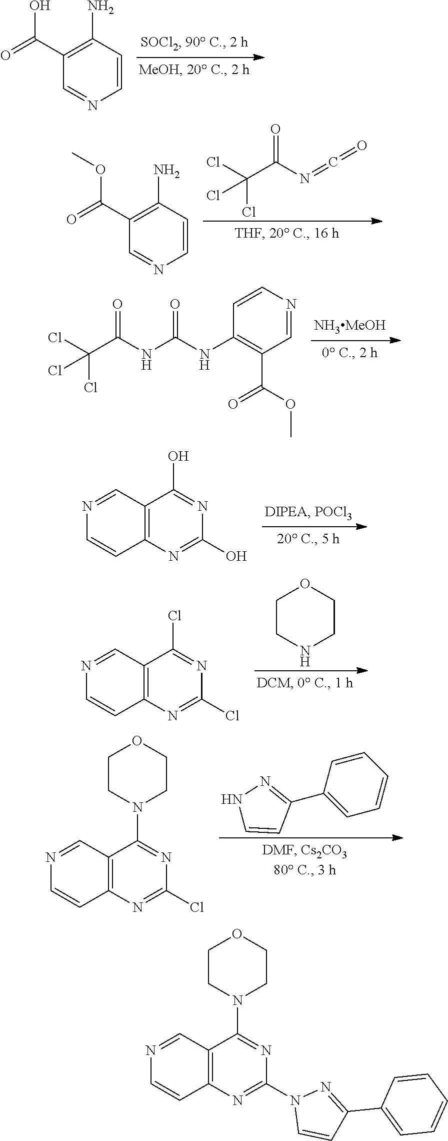

### Step 1: Synthesis of methyl 4-aminonicotinate

A suspension of 4-aminonicotinic acid (5.0 g, 36.2 mmol) in thionyl chloride (10 ml) was stirred at 90° C. for 1 h. The reaction mixture was concentrated to afford 4-aminonicotinoyl chloride (5.1 g, 90%) a as yellow solid. This was dissolved in dry methanol (50 mL) and stirred at 20° C. for 2 h, The reaction mixture was concentrated, aqueous sodium carbonate solution (200 mL) was added and the mixture was stirred further at 20° C. for 0.5 h and extracted with dichloromethane (150 mL*2), The combined organic layer was dried over anhydrous sodium sulfate, filtered and concentrated under reduced pressure to give methyl 4-aminonicotinate (5.1 g, 93%) as yellow solid. LCMS (ESI) m/z: 153.1 [M+H]⁺.

### Step 2: Synthesis of methyl 4-(3-(2,2,2-trichloroacetyl)ureido)nicotinate

To a solution of methyl 4-aminonicotinate (1.0 g, 6.57 mmol) in tetrahydrofuran (20 mL) at 0° C., 2,2,2-trichloroacetyl isocyanate (2.6 g, 13.8 mmol) was added dropwise and then stirred at 20° C. for 20 h. The resultant precipitate was collected by filtration and washed with methanol to give methyl 4-(3-(2,2,2-trichloroacetyl)ureido)nicotinate (1.5 g, 67%) as yellow solid. LCMS (ESI) m/z: 341.9 [M+H]⁺.

### Step 3: Synthesis of pyrido[4,3-d]pyrimidine-2,4-diol

To an ice cold solution of methyl 4-(3-(2,2,2-trichloroacetyl)ureido)nicotinate (750 mg, 2.2 mmol) in anhydrous methanol (7 mL) was added ammonia/methanol (2 mL). The suspension was stirred at 0° C. for 2 h at which time a yellow solid precipitated. The solid was collected by filtration and washed with methanol to give pyrido[4,3-d]pyrimidine-2,4-diol (300 mg, 84%) as yellow solid. ¹H NMR (500 MHz, DMSO-d6) δ 11.43 (s, 2H), 8.92 (s, 1H), 8.58 (d, J=5.7 Hz, 1H), 7.08 (d, J=5.9 Hz, 1H). LCMS (ESI) m/z: 164.1 [M+H]⁺.

### Step 4: Synthesis of 2,4-dichloropyrido[4,3-d]pyrimidine

To a suspension of pyrido[4,3-d]pyrimidine-2,4-diol (300 mg, 1.84 mmol) in phosphorus oxychloride (6 mL) was added N,N-diisopropylethylamine (3 mL) and then the mixture was stirred at 20° C. for 5 h. It was then concentrated to afford 2,4-dichloropyrido[4,3-d]pyrimidine (368 mg, 99%) as red solid. LCMS (ESI) m/z: 202.2 [M+H]⁺.

### Step 5: Synthesis of 4-(2-chloropyrido[4,3-d]pyrimidin-4-yl)morpholine

A mixture of 2,4-dichloropyrido[4,3-d]pyrimidine (367 mg, 0.76 mmol) and morpholine (662 mg, 183 mmol) in dichloromethane (20 mL) was stirred at 0° C. for 1 h. It was then diluted with dichloromethane (50 mL) and washed with water (50 mL). The organic layer was concentrated and purified by Combi-Flash (Biotage, 40 g silica gel, methanol in dichloromethane from 1% to 6%) to obtain 4-(2-chloropyrido[4,3-d]pyrimidin-4-yl)morpholine (300 mg) as yellow solid. LCMS (ESI) m/z: 251.1 [M+H]⁺.

### Step 6: Synthesis of 4-(2-(3-phenyl-1H-pyrazol-1-yl)pyrido[4,3-d]pyrimidin-4-yl)morpholine

To a solution of 4-(2-chloropyrido[4,3-d]pyrimidin-4-yl)morpholine (250 mg, 1.0 mmol) in DMF (2 mL) were added 3-phenyl-1H-pyrazole (35 mg, 0.24 mmol) and cesium carbonate (130 mg, 0.4 mmol) and the resultant mixture was stirred at 80° C. under nitrogen for 3 h. The resultant mixture was filtered and the filtrate was subjected to prep-HPLC (BOSTON pHlex ODS 10 um 21.2×250 mm 120A. The mobile phase was acetonitrile/0.1% Ammonium bicarbonate) to obtain 4-(2-(3-phenyl-1H-pyrazol-1-yl)pyrido[4,3-d]pyrimidin-4-yl)morpholine (8.5 mg, 2.4%) as white solid. ¹H NMR (500 MHz, Chloroform-d) δ 9.26 (d, J=0.8 Hz, 1H), 8.70 (d, J=5.8 Hz, 1H), 8.66 (d, J=2.7 Hz, 1H), 8.04 (d, J=4.8 Hz, 2H), 7.81 (d, J=5.8, 0.8 Hz, 1H), 7.48-7.42 (m, 2H), 7.40-7.36 (m, 1H), 6.84 (d, J=2.7 Hz, 1H), 4.13 (t, J=4.0 Hz, 4H), 3.98 (t, J=4.0 Hz, 4H). LCMS (ESI) m/z: 359.3 [M+H]⁺.

### Synthesis of (tert-butyl 2,2-dimethyl-4-[1-(4-morpholinopyrido[3,2-d]pyrimidin-2-yl)pyrazol-3-yl]piperidine-1-carboxylate (Compound 54) and 4-[2-[3-(2,2-dimethyl-4-piperidyl)pyrazol-1-yl]pyrido[3,2-d]pyrimidin-4-yl]morpholine (Compound 49)

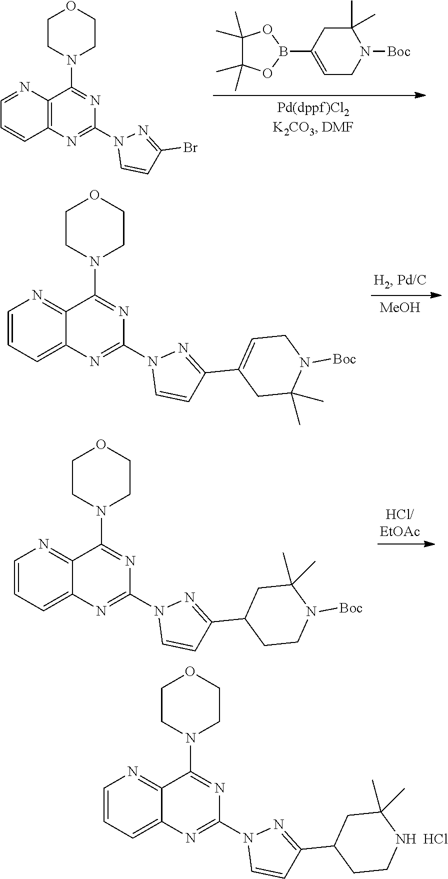

### Step 1: Synthesis of tert-butyl 6,6-dimethyl-4-[1-(4-morpholinopyrido[3,2-d]pyrimidin-2-yl)pyrazol-3-yl]-2,5-dihydropyridine-1-carboxylate

To a solution of 4-[2-(3-bromopyrazol-1-yl)pyrido[3,2-d]pyrimidin-4-yl]morpholine (150 mg, 415.11 umol) in dioxane (1 mL) and H₂O (0.2 mL), were added tert-butyl 6,6-dimethyl-4-(4,4,5,5-tetramethyl-1,3,2-dioxaborolan-2-yl)-2,5-dihydropyridine-1-carboxylate (140 mg, 415 umol), Pd(dppf)Cl₂ (32 mg, 42 umol), and K₂CO₃ (143 mg, 1.04 mol). The resultant mixture was stirred at 60° C. for 6 h under nitrogen. The mixture was then filtered and the filtrated was concentrated to obtain tert-butyl 6,6-dimethyl-4-[1-(4-morpholinopyrido[3,2-d]pyrimidin-2-yl)pyrazol-3-yl]-2,5-dihydropyridine-1-carboxylate (80 mg, 39%) as pale yellow solid. 1H NMR (400 MHz, CHLOROFORM-d) δ=8.62 (d, J=3.0 Hz, 1H), 8.54 (d, J=2.6 Hz, 1H), 8.24-8.18 (m, 1H), 7.59 (dd, J=4.1, 8.5 Hz, 1H), 6.59 (d, J=2.6 Hz, 1H), 6.44 (t, J=3.9 Hz, 1H), 4.62 (bs, 4H), 4.11 (bs, 2H), 3.96-3.91 (m, 4H), 2.04 (s, 2H), 1.52-1.47 (m, 15H); LCMS (ESI) m/z: 492.2 [M+H]⁺.

### Step 2: Synthesis of tert-butyl 2,2-dimethyl-4-[1-(4-morpholinopyrido[3,2-d]pyrimidin-2-yl)pyrazol-3-yl]piperidine-1-carboxylate

To a solution of tert-butyl 6,6-dimethyl-4-[1-(4-morpholinopyrido[3,2-d]pyrimidin-2-yl)pyrazol-3-yl]-2,5-dihydropyridine-1-carboxylate (55 mg, 112 umol) in MeOH (0.5 mL), was added Pd/C (100 mg, 10% purity), and the mixture was stirred at 20° C. for 40 min under hydrogen atmosphere (15 Psi). The mixture was filtered and the filtrate was evaporated to obtain tert-butyl 2,2-dimethyl-4-[1-(4-morpholinopyrido[3,2-d]pyrimidin-2-yl)pyrazol-3-yl]piperidine-1-carboxylate (80 mg) as white solid. 1H NMR (400 MHz, METHANOL-d4) δ 8.69 (dd, J=4.1, 1.6 Hz, 1H), 8.61 (d, J=2.6 Hz, 1H), 8.16 (dd, J=8.6, 1.5 Hz, 1H), 7.72 (dd, J=8.6, 4.2 Hz, 1H), 6.45 (d, J=2.6 Hz, 1H), 4.65 (bs, 4H), 3.97-3.85 (m, 5H), 3.27-3.11 (m, 2H), 2.17-2.05 (m, 1H), 1.97-1.88 (m, 1H), 1.86-1.71 (m, 2H), 1.57 (s, 3H), 1.48 (s, 9H), 1.44 (s, 3H). LCMS (ESI) for (C26H35N7O3) [M+H]+: 494.3.

### Step 3: Synthesis of 4-[2-[3-(2,2-dimethyl-4-piperidyl)pyrazol-1-yl]pyrido[3,2-d]pyrimidin-4-yl]morpholine

To a solution of tert-butyl 2,2-dimethyl-4-[1-(4-morpholinopyrido[3,2-d]pyrimidin-2-yl)pyrazol-3-yl]piperidine-1-carboxylate (40 mg, 81 umol) in MeOH (1 mL), was added HCl/MeOH (4 M, 2.40 mL) and the mixture was stirred at 20° C. for 1 h. The mixture was filtered and the filtrate was purified by prep-HPLC (Phenomenex luna C18 80*40 mm*3 um column; 10%-30% acetonitrile in a 0.04% hydrochloric acid solution in water, 7 min gradient) to obtain 4-[2-[3-(2,2-dimethyl-4-piperidyl)pyrazol-1-yl]pyrido[3,2-d]pyrimidin-4-yl]morpholine (5 mg, 15%) as white solid. ¹H NMR (400 MHz, METHANOL-d4) δ 8.81 (d, J=4.0 Hz, 1H), 8.73 (bs, 1H), 8.33 (d, J=7.6 Hz, 1H), 7.87 (dd, J=8.0, 4.1 Hz, 1H), 6.63 (bs, 1H), 4.75 (bs, 4H), 3.93 (t, J=4.4 Hz, 4H), 3.45-3.35 (m, 3H), 2.31 (d, J=14.5 Hz, 1H), 2.24-2.15 (m, 1H), 2.02-1.87 (m, 2H), 1.53 (s, 3H), 1.50 (s, 3H). LCMS (ESI) for (C21H27N7O) [M+H]+: 394.2.

### Synthesis of 2-(4-morpholinopyrido[3,2-d]pyrimidin-2-yl)-5-phenyl-4H-1,2,4-triazol-3-one (Compound 50)

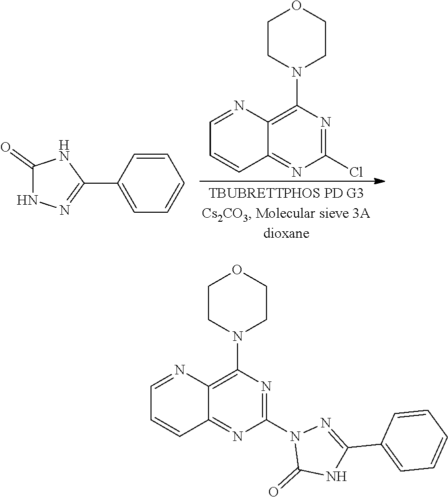

To a solution of 4-(2-chloropyrido[3,2-d]pyrimidin-4-yl)morpholine (200 mg, 798 umol), 3-phenyl-1,4-dihydro-1,2,4-triazol-5-one (154 mg, 957 umol), Cs₂CO₃ (520 mg, 1.60 mmol) and Molecular sieve 3A (20 mg, 1.00 eq) in dioxane (3 mL) was added TBUBRETTPHOS PD G3 (68 mg, 80 mol) and stirred for 16 h at 80° C. under nitrogen atmosphere. The resultant mixture was filtered and the filtrate was subjected to prep-HPLC (Waters Xbridge Prep OBD C18 150*40 mm*10 um column; 20%-50% acetonitrile in an 10 mM NH₄HCO₃ in water, 8 min gradient) to obtain 2-(4-morpholinopyrido[3,2-d]pyrimidin-2-yl)-5-phenyl-4H-1,2,4-triazol-3-one (76 mg, 25%) as white solid. ¹H NMR (400 MHz, DMSO-d₆+1 drop HCl) δ=8.74 (d, J=4 Hz, 1H), 8.57 (d, J=8.4 Hz, 1H), 8.00-7.98 (m, 2H), 7.89-7.86 (m, 1H), 7.52-7.45 (m, 3H), 5.05 (bs, 2H), 4.28 (bs, 2H), 3.79 (s, 4H). LCMS (ESI) for (C19H17N7O2) [M+H]⁺: 376.2

### Synthesis of 4-[7-cyclopropyl-2-(3-phenylpyrazol-1-yl)pyrido[3,2-d]pyrimidin-4-yl]morpholine (Compound 57)

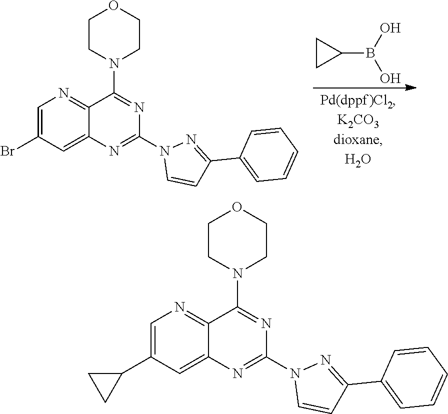

To a solution of 4-[7-bromo-2-(3-phenylpyrazol-1-yl)pyrido[3,2-d]pyrimidin-4-yl]morpholine (0.1 g, 229 umol) in dioxane (2 mL) and H₂O (0.4 mL) were added cyclopropyl boronic acid (39 mg, 457 umol), K₂CO₃ (79 mg, 572 umol) and Pd(dppf)Cl₂ (8 mg, 11 umol). The resultant mixture stirred at 80° C. for 16 h under nitrogen atmosphere. 10 mL of water was added to the mixture and it was extracted with ethyl acetate (20 mL*2). The combined organic layers were washed with brine (15 mL), dried over Na₂SO₄ and concentrated. The resultant residue was subjected to prep-HPLC (Welch Xtimate C18 150*25 5u column, 20-50% acetonitrile in an 0.04% hydrochloric acid solution in water, 8 min gradient) to afford 4-[7-cyclopropyl-2-(3-phenylpyrazol-1-yl)pyrido[3,2-d]pyrimidin-4-yl]morpholine (51 mg, 56%) as white solid. ¹H NMR (400 MHz, DMSO-d6) δ=8.88 (d, J=2.6 Hz, 1H), 8.64 (d, J=2.3 Hz, 1H), 8.08-8.00 (m, 2H), 7.92 (d, J=2.1 Hz, 1H), 7.55-7.40 (m, 3H), 7.19 (d, J=2.8 Hz, 1H), 4.63 (bs, 4H), 3.83 (t, J=4.4 Hz, 4H), 2.26-2.15 (m, 1H), 1.26-1.16 (m, 2H), 1.04-0.92 (m, 2H). LCMS (ESI) for C23H22N6O [M+H]⁺: 399.2.

### Synthesis of 4-[2-(3-phenylpyrazol-1-yl)-6-tetrahydropyran-4-yl-pyrido[3,2-d]pyrimidin-4-yl]morpholine (Compound 58)

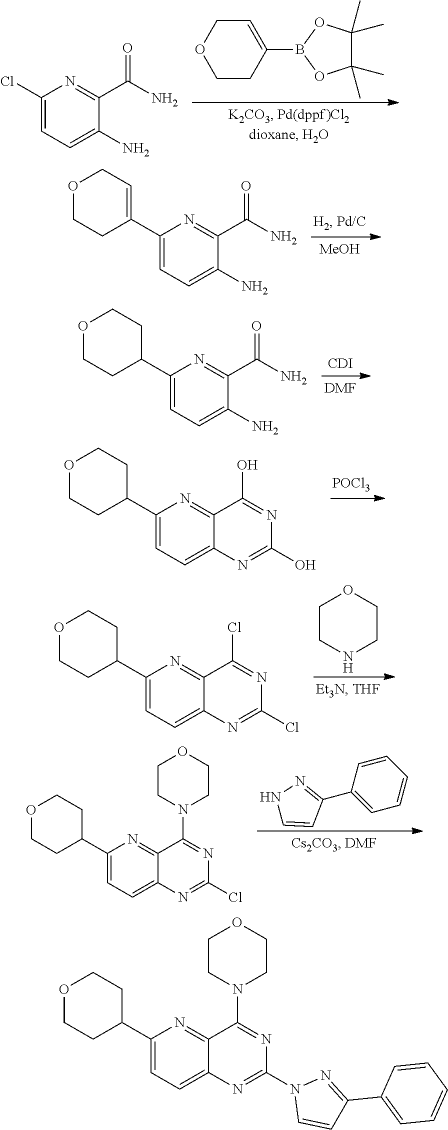

### Step 1: Synthesis of 3-amino-6-(3,6-dihydro-2H-pyran-4-yl)pyridine-2-carboxamide

To a solution of 3-amino-6-chloro-pyridine-2-carboxamide (3 g, 17.48 mmol) in dioxane (50 mL) and water (5 mL) were added 2-(3,6-dihydro-2H-pyran-4-yl)-4,4,5,5-tetramethyl-1,3,2-dioxaborolane (7.35 g, 34.97 mmol), K₂CO₃ (6.04 g, 43.71 mmol) and Pd(dppf)Cl₂ (640 mg, 874 umol). The resultant mixture was stirred at 80° C. for 16 h under nitrogen atmosphere. Water (20 mL) and EtOAc (50 mL) were added to the reaction mixture, filtered and filtrate was concentrated to obtain 3-amino-6-(3,6-dihydro-2H-pyran-4-yl)pyridine-2-carboxamide (2.2 g, 57%) as black solid. ¹H NMR (400 MHz, DMSO-d6) δ=7.86 (bs, 1H), 7.49 (d, J=8.8 Hz, 1H), 7.33 (bs, 1H), 7.13 (d, J=8.8 Hz, 1H), 6.87 (bs, 2H), 6.51 (bs, 1H), 4.22 (d, J=2.4 Hz, 2H), 3.79 (t, J=5.4 Hz, 2H), 2.58-2.52 (m, 2H).

### Step 2: Synthesis of 3-amino-6-tetrahydropyran-4-yl-pyridine-2-carboxamide

To a solution of 3-amino-6-(3,6-dihydro-2H-pyran-4-yl)pyridine-2-carboxamide (2 g, 9.12 mmol) in MeOH (60 mL) was added Pd/C (1 g, 10% purity) under argon. The suspension was degassed under vacuum and purged with hydrogen several times. The mixture was stirred under hydrogen balloon (15 psi) at 20° C. for 16 h. The reaction mixture was filtered and the filtrate was concentrated in vacuo to obtain 3-amino-6-tetrahydropyran-4-yl-pyridine-2-carboxamide (1.6 g, 79%) as pale yellow solid. LCMS (ESI) m/z: 222.1 [M+H]+

### Step 3: Synthesis of 6-tetrahydropyran-4-ylpyrido[3,2-d]pyrimidine-2,4-diol

To a solution of 3-amino-6-tetrahydropyran-4-yl-pyridine-2-carboxamide (0.8 g, 3.62 mmol) in DMF (10 mL) was added CDI (879 mg, 5.42 mmol). The mixture was stirred at 90° C. for 16 h and cooled. The reaction mixture was filtered and the filtrate was concentrated in vacuo to obtain 6-tetrahydropyran-4-ylpyrido[3,2-d]pyrimidine-2,4-diol (0.3 g, 34%) as pale brown solid. LCMS (ESI) m/z: 248.1 [M+H]+

### Step 4: Synthesis of 2,4-dichloro-6-tetrahydropyran-4-yl-pyrido[3,2-d]pyrimidine

A mixture of 6-tetrahydropyran-4-ylpyrido[3,2-d]pyrimidine-2,4-diol (0.3 g, 1.21 mmol) in POCl₃ (4 mL) was stirred at 120° C. for 6 h. It was concentrated and 10 mL ice water was added. After stirring at 20° C. for 0.5 h, the mixture was basified by 2N NaOH (4 mL) and the reaction mixture was extracted with DCM (20 mL*2). The combined organic layers were washed with brine (15 mL), dried over Na₂SO₄ and concentrated to obtain 2,4-dichloro-6-tetrahydropyran-4-yl-pyrido[3,2-d]pyrimidine (200 mg) as brown solid. LCMS (ESI) m/z: 284.0 [M+H]+

### Step 5: Synthesis of 4-(2-chloro-6-tetrahydropyran-4-yl-pyrido[3,2-d]pyrimidin-4-yl)morpholine

To a solution of 2,4-dichloro-6-tetrahydropyran-4-yl-pyrido[3,2-d]pyrimidine (190 mg, 669 umol) in THE (8 mL) were added morpholine (61 mg, 702 umol) and Et₃N (71 mg, 702 umol) at 0° C. The resultant mixture was warmed up and stirred at 20° C. for 1 h. It was concentrated and the residue was dissolved in 30 mL chloroform, washed with a saturated aqueous solution of sodium bicarbonate (10 mL), dried over Na₂SO₄ and filtered. The resultant solution was concentrated to obtain 4-(2-chloro-6-tetrahydropyran-4-yl-pyrido[3,2-d]pyrimidin-4-yl)morpholine (0.2 g, crude) as pale brown solid.

### Step 6: Synthesis of 4-[2-(3-phenylpyrazol-1-yl)-6-tetrahydropyran-4-yl-pyrido[3,2-d]pyrimidin-4-yl]morpholine

To a solution of 4-(2-chloro-6-tetrahydropyran-4-yl-pyrido[3,2-d]pyrimidin-4-yl)morpholine (190 mg, 568 umol) in DMF (5 mL) were added 3-phenyl-1H-pyrazole (90 mg, 624 umol) and Cs₂CO₃ (370 mg, 1.14 mmol). The mixture was stirred at 100° C. for 16 h and then 15 mL of water was added to the reaction mixture. It was extracted with ethyl acetate (30 mL*2), washed with brine (15 mL), dried over Na₂SO₄ and concentrated. The crude product was purified by prep-HPLC (Nano-micro Kromasil C18 100*40 3u column; 1-42% acetonitrile in an a 0.04% hydrochloric acid solution in water, 8 min gradient) to obtain 4-[2-(3-phenylpyrazol-1-yl)-6-tetrahydropyran-4-yl-pyrido[3,2-d]pyrimidin-4-yl]morpholine (143 mg, 51%) as pale yellow solid. ¹H NMR (400 MHz, CHLOROFORM-d) δ=9.76 (d, J=8.8 Hz, 1H), 8.72 (bs, 1H), 8.19 (d, J=7.6 Hz, 2H), 7.69 (d, J=8.8 Hz, 1H), 7.51-7.33 (m, 3H), 7.00-6.93 (m, 1H), 5.28 (bs, 2H), 4.43 (bs, 2H), 4.14 (d, J=10.7 Hz, 2H), 4.03 (bs, 4H), 3.60 (dt, J=11.1, 3.4 Hz, 2H), 3.19-3.06 (m, 1H), 2.01-1.78 (m, 4H). LCMS (ESI) for C25H26N6O2 [M+H]+: 443.2.

### Synthesis of 4-[2-[3-(3-fluorophenyl)pyrazol-1-yl]-7-tetrahydropyran-4-yl-pyrido[3,2-d]pyrimidin-4-yl]morpholine (Compound 69)

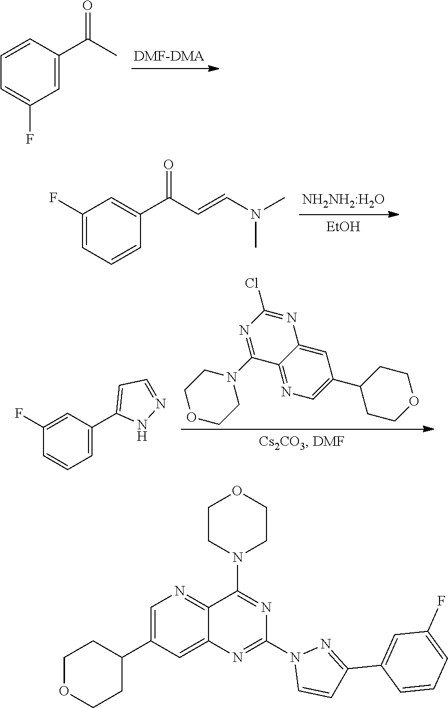

### Step 1: Synthesis of (E)-3-(dimethylamino)-1-(3-fluorophenyl)prop-2-en-1-one

A solution of 1-(3-fluorophenyl)ethanone (1 g, 7.24 mmol) in DMF-DMA (7 mL) was stirred at 100° C. for 12 h and concentrated. The resultant crude product was purified by flash column chromatography (ISCO 40 g silica, 0-40% ethyl acetate in petroleum ether, gradient over 20 min) to obtain (E)-3-(dimethylamino)-1-(3-fluorophenyl)prop-2-en-1-one (850 mg, 61%) as yellow solid. ¹H NMR (400 MHz, DMSO-d₆) δ=7.73 (d, J=11.6 Hz, 2H), 7.64 (d, J=10 Hz, 1H), 7.46 (dd, J=7.6, 1.6 Hz, 1H), 7.44-7.31 (m, 1H), 5.83 (d, J=12 Hz, 1H), 3.1 (s, 1H), 2.9 (s, 3H).

### Step 2: Synthesis of 5-(3-fluorophenyl)-1H-pyrazole

To a solution of (E)-3-(dimethylamino)-1-(3-fluorophenyl)prop-2-en-1-one (520 mg, 2.69 mmol) in EtOH (1 mL) was added hydrazine;hydrate (250 mg, 5.38 mmol) and the mixture was stirred at 15° C. for 10 h. 5 mL of water and 5 mL of ethyl acetate were added to the reaction mixture, the organic layer separated and aqueous layer was extracted with EtOAc (5 mL*3). The combined organic layers were washed with brine (5 mL*3), dried over Na₂SO₄, filtered and concentrated to obtain 5-(3-fluorophenyl)-1H-pyrazole (480 mg) as yellow solid. LCMS (ESI) m/z: 163.1 [M+H]⁺

### Step 3: Synthesis of 4-[2-[3-(3-fluorophenyl)pyrazol-1-yl]-7-tetrahydropyran-4-yl-pyrido[3,2-d]pyrimidin-4-yl]morpholine

A mixture of 4-(2-chloro-7-tetrahydropyran-4-yl-pyrido[3,2-d]pyrimidin-4-yl)morpholine (100 mg, 299 umol), 5-(3-fluorophenyl)-1H-pyrazole (53 mg, 329 umol) and Cs₂CO₃ (195 mg, 597 umol) in 1 mL of DMF was stirred at 100° C. for 10 h. The mixture was filtered and the filtrate was concentrated and subjected to prep-HPLC (Waters X bridge 150*30 mm*5 uM column; 30-60% acetonitrile in an a 10 mM ammonium bicarbonate solution in water, 8 min gradient) to afford 4-[2-[3-(3-fluorophenyl)pyrazol-1-yl]-7-tetrahydropyran-4-yl-pyrido[3,2-d]pyrimidin-4-yl]morpholine (54 mg, 39%) as white solid. 1H NMR (400 MHz, DMSO-d6) δ=8.80 (d, J=2.4 Hz, 1H), 8.72 (d, J=2 Hz, 1H), 8.00 (s, 1H), 7.83 (d, J=7.6 Hz, 1H), 7.76 (d, J=10.4 Hz, 1H), 7.55 (q, 1H), 7.26-7.22 (m, 1H), 7.15 (d, J=2.8 Hz, 1H), 4.57 (bs, 4H), 4.02-3.99 (m, 2H), 3.84-3.82 (m, 4H), 3.83-3.50 (m, 2H), 3.48-3.06 (m, 1H), 1.85-1.77 (m, 4H). LCMS (ESI for C25H25FN6O2) [M+H]⁺: 461.2.

### Synthesis of tert-butyl 3-[[1-(4-morpholinopyrido[3,2-d]pyrimidin-2-yl)pyrazol-3-yl]methyl]pyrrolidine-1-carboxylate (Compound 70) and 4-[2-[3-(pyrrolidin-3-ylmethyl)pyrazol-1-yl]pyrido[3,2-d]pyrimidin-4-yl]morpholine (Compound 52)

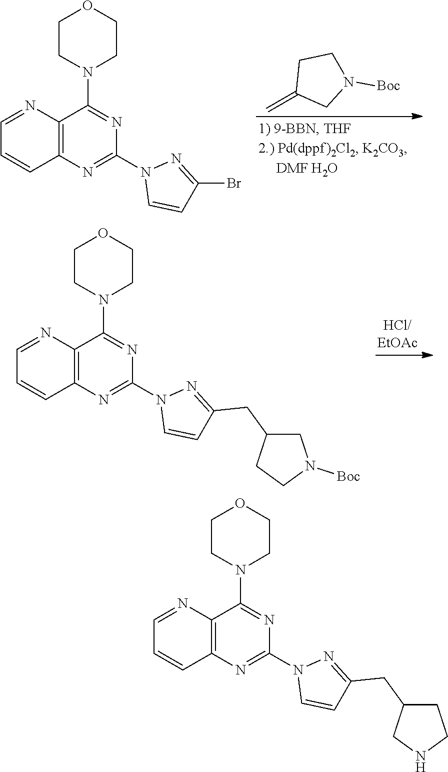

### Step 1: Synthesis of tert-butyl 3-[[1-(4-morpholinopyrido[3,2-d]pyrimidin-2-yl)pyrazol-3-yl]methyl]pyrrolidine-1-carboxylate

A mixture of tert-butyl 3-methylenepyrrolidine-1-carboxylate (507 mg, 2.77 mmol) and 9-BBN (0.5M in THF, 5.54 mL) was stirred at 80° C. for 1 h. It was then cooled to 20° C. and to the resultant solution were added 4-[2-(3-bromopyrazol-1-yl)pyrido[3,2-d]pyrimidin-4-yl]morpholine (0.5 g, 1.38 mmol), Pd(dppf)Cl₂·CH₂Cl₂ (57 mg, 69 umol), K₂CO₃ (287 mg, 2.08 mmol), DMF (5 mL) and water (0.5 mL). The resulting mixture was heated at 80° C. for 15 h. 15 mL of water was then added to the reaction mixture and it was extracted with ethyl acetate (30 mL*2). The combined organic layers were washed with brine (15 mL), dried over Na₂SO₄ and concentrated. The crude product was purified by prep-HPLC (Phenomenex Gemini-NX 150*30 5u column; 20-50% acetonitrile in an a 10 mM ammonium bicarbonate solution in water, 8 min gradient) to obtain tert-butyl 3-[[1-(4-morpholinopyrido[3,2-d]pyrimidin-2-yl)pyrazol-3-yl]methyl]pyrrolidine-1-carboxylate (150 mg, 23%) as white solid. ¹H NMR (400 MHz, CHLOROFORM-d) δ=8.63 (d, J=2.6 Hz, 1H), 8.50 (d, J=7.1 Hz, 1H), 8.27 (dd, J=8.6, 1.6 Hz, 1H), 7.59 (dd, J=8.6, 4.1 Hz, 1H), 6.30 (d, J=2.5 Hz, 1H), 4.61 (bs, 4H), 3.93 (t, J=4.8 Hz, 4H), 3.68-3.39 (m, 2H), 3.36-3.20 (m, 1H), 3.14-2.97 (m, 1H), 2.95-2.80 (m, 2H), 2.70-2.48 (m, 1H), 2.09-1.97 (m, 1H), 1.72-1.63 (m, 1H), 1.46 (s, 9H). LCMS (ESI) for C24H31N7O3 [M+H]+: 466.3.

### Step 5: Synthesis of 4-[2-[3-(pyrrolidin-3-ylmethyl)pyrazol-1-yl]pyrido[3,2-d]pyrimidin-4-yl]morpholine

A mixture of tert-butyl 3-[[1-(4-morpholinopyrido[3,2-d]pyrimidin-2-yl)pyrazol-3-yl]methyl]pyrrolidine-1-carboxylate (120 mg, 258 umol) in 4M HCl/EtOAc (10 mL) was stirred at 25° C. for 1 h. The reaction mixture was concentrated and the crude product was purified by prep-HPLC (Phenomenex luna C18 80*40 3u column; 8-48% acetonitrile in an a 0.04% hydrochloric acid solution in water, 7 min gradient) to obtain 4-[2-[3-(pyrrolidin-3-ylmethyl)pyrazol-1-yl]pyrido[3,2-d]pyrimidin-4-yl]morpholine·HCl (82 mg, 79%) as white solid. ¹H NMR (400 MHz, METHANOL-d4) δ=8.87 (d, J=3.3 Hz, 1H), 8.77 (d, J=2.4 Hz, 1H), 8.42 (d, J=8.5 Hz, 1H), 7.93 (dd, J=8.6, 4.3 Hz, 1H), 6.67 (d, J=2.4 Hz, 1H), 5.52-4.90 (m, 2H), 4.82-4.24 (m, 2H), 3.96 (t, J=4.4 Hz, 4H), 3.54 (dd, J=11.5, 7.6 Hz, 1H), 3.49-3.41 (m, 1H), 3.36-3.3 (m, 1H), 3.11-2.96 (m, 3H), 2.93-2.77 (m, 1H), 2.33-2.15 (m, 1H), 1.83 (qd, J=13.1, 8.7 Hz, 1H). LCMS (ESI) for C19H23N7O [M+H]+: 366.2.

### Synthesis of 4-[2-(1H-indazol-3-yl)pyrido[3,2-d]pyrimidin-4-yl]morpholine (Compound 71)

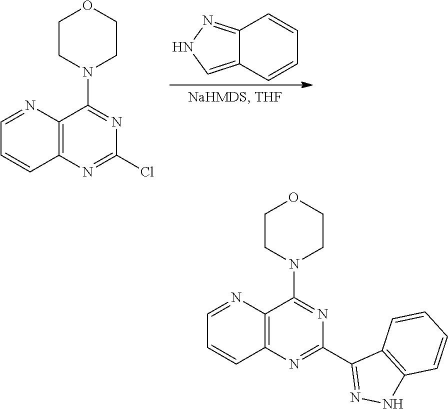 [SMILES: Clc1nc(N2CCOCC2)c2ncccc2n1 ; c1ccc2n[nH]cc2c1 ; c1cnc2c(N3CCOCC3)nc(-c3n[nH]c4ccccc34)nc2c1]

A solution of 2H-indazole (94 mg, 798 umol) and NaHMDS (1 M, 1.60 mL) in THE (2 mL) was stirred for 0.5 h at 0° C., followed by the addition of 4-(2-chloropyrido[3,2-d]pyrimidin-4-yl)morpholine (200 mg, 798 umol). The resultant mixture was further stirred at 15° C. for 14 h. The mixture was concentrated and the crude product obtained was purified by prep-HPLC (Waters Xbridge BEH 018 100*30 mm*10 um column; 35-60% acetonitrile in an 10 mM ammonium bicarbonate in water, 8 ml gradient) to obtain 4-[2-(1H-indazol-3-yl)pyrido[3,2-d]pyrimidin-4-yl]morpholine (34 mg, 13%) as pale yellow solid. ¹H NMR (400 MHz, DMSO-d6) 9.25 (s, 1H), 8.50-8.49 (m, 1H), 7.80-7.75 (m, 3H), 7.63-7.61 (m, 2H), 7.25-7.22 (m, 1H), 4.36 (bs, 4H), 3.75 (t, J=4.8 Hz, 4H); LCMS (ESI for C18H16N6O) [M+H]+: 333.2. (NOE experiments confirmed the regioselectivity and the product).

The following compounds were prepared by synthetic protocols known to one of skill in the art.

| Name | Structure | NMR, MS | # |
|---|---|---|---|
| 4-[2-[3-(4- pyridyl)pyrazol-1-yl]-7- tetrahydropyran-4-yl- pyrido[3,2-d]pyrimidin- 4-yl]morpholine | 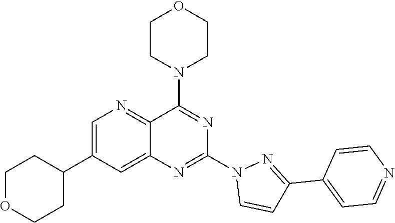 [SMILES: c1cc(-c2ccn(-c3nc(N4CCOCC4)c4ncc(C5CCOCC5)cc4n3)n2)ccn1] | ¹H NMR (400 MHz, CHLOROFORM-d) δ = 8.74 − 8.65 (m, 3H), 8.58 (d, J = 2.3 Hz, 1H), 8.08 (d, J = 2.1 Hz, 1H), 7.95 − 7.86 (m, 2H), 6.89 (d, J = 2.8 Hz, 1H), 4.66 (br s, 4H), 4.16 (dd, J = 2.8, 10.5 Hz, 2H), 4.01 − 3.91 (m, 4H), 3.61 (dt, J = 3.2, 11.2 Hz, 2H), 3.05 − 2.90 (m, 1H), 1.99 − 1.82 (m, 4H). LCMS (ESI) for C24H25N7O2 [M + H]⁺: 444.3. | 59 |
| 4-[2-[3-(3- pyridyl)pyrazol-1-yl]-7- tetrahydropyran-4-yl- pyrido[3,2-d]pyrimidin- 4-yl]morpholine | 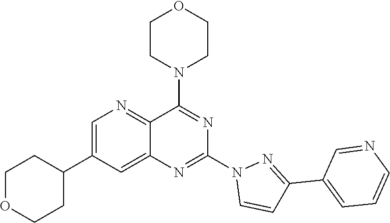 [SMILES: c1cncc(-c2ccn(-c3nc(N4CCOCC4)c4ncc(C5CCOCC5)cc4n3)n2)c1] | ¹H NMR (400 MHz, CHLOROFORM-d) δ = 9.19 (d, J = 1.6 Hz, 1H), 8.67 (d, J = 2.8 Hz, 1H), 8.61 (dd, J = 4.8, 1.6 Hz, 1H), 8.57 (d, J = 2.3 Hz, 1H), 8.37 (td, J = 8.0, 1.9 Hz, 1H), 8.08 (d, J = 2.0 Hz, 1H), 7.43 − 7.32 (m, 1H), 6.86 (d, J = 2.8 Hz, 1H), 4.65 (bs, 4H), 4.15 (dd, J = 10.4, 2.8 Hz, 2H), 3.94 (t, J = 4.4 Hz, 4H), 3.60 (dt, J = 11.2, 3.3 Hz, 2H), 2.98 (hept, J = 5.2 Hz, 1H), 2.00 − 1.80 (m, 4H). LCMS (ESI) for C24H25N7O2 [M + H]⁺: 444.3. | 61 |
| 4-[4-morpholino-2-(3- phenylpyrazol-1- yl)pyrido[3,2- d]pyrimidin-7- yl]morpholine | 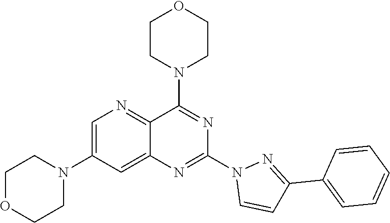 [SMILES: c1ccc(-c2ccn(-c3nc(N4CCOCC4)c4ncc(N5CCOCC5)cc4n3)n2)cc1] | ¹H NMR (400 MHz, CHLOROFORM-d) δ = 8.63 (d, J = 2.6 Hz, 1H), 8.44 (d, J = 2.9 Hz, 1H), 8.09 − 7.97 (m, 2H), 7.47 − 7.40 (m, 3H), 7.39 − 7.32 (m, 1H), 6.80 (d, J = 2.8 Hz, 1H), 4.58 (bs, 4H), 4.00 − 3.86 (m, 8H), 3.44 − 3.35 (m, 4H). LCMS (ESI) for C24H25N7O2 [M + H]⁺: 444.2. | 62 |
| 4-[2-(3-pyrimidin-5- ylpyrazol-1-yl)-7- tetrahydropyran-4-yl- pyrido[3,2-d]pyrimidin- 4-yl]morpholine | 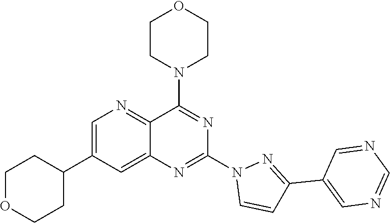 [SMILES: c1ncc(-c2ccn(-c3nc(N4CCOCC4)c4ncc(C5CCOCC5)cc4n3)n2)cn1] | ¹H NMR (400 MHz, CHLOROFORM-d) δ = 9.34 (s, 2H), 9.22 (s, 1H), 8.71 (d, J = 2.8 Hz, 1H), 8.59 (d, J = 2.3 Hz, 1H), 8.08 (d, J = 2.0 Hz, 1H), 6.87 (d, J = 2.8 Hz, 1H), 4.65 (bs, 4H), 4.15 (dd, J = 10.4, 2.6 Hz, 2H), 3.95 (t, J = 4.4 Hz, 4H), 3.60 (dt, J = 11.2, 3.4 Hz, 2H), 2.99 (hept, J = 5.2 Hz, 1H), 1.99 − 1.83 (m, 4H). LCMS (ESI) for C23H24N8O2 [M + H]⁺: 445.3. | 64 |
| 4-[2-(3-pyrimidin-4- ylpyrazol-1-yl)-7- tetrahydropyran-4-yl- pyrido[3,2-d]pyrimidin- 4-yl]morpholine | 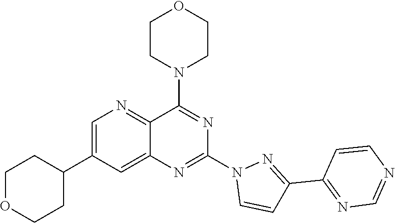 [SMILES: c1cc(-c2ccn(-c3nc(N4CCOCC4)c4ncc(C5CCOCC5)cc4n3)n2)ncn1] | ¹H NMR (400 MHz, DMSO-d6) δ = 9.27 (s, 1H), 8.97 − 8.92 (m, 2H), 8.88 − 8.75 (m, 1H), 8.13 − 8.12 (m, 1H), 8.03 (s, 1H), 7.23 (s, 1H), 4.57 (bs, 4H), 4.03 − 4.00 (m, 2H), 3.84 (m, 4H), 3.53 − 3.51 (m, 2H), 3.08 (m, 1H), 1.85 − 1.81 (m, 4H). LCMS (ESI) for C23H24N8O2 [M + H]⁺: 445.2. | 65 |
| 4-[2-(3-phenylpyrazol- 1-yl)-7-piperazin-1-yl- pyrido[3,2-d]pyrimidin- 4-yl]morpholine | 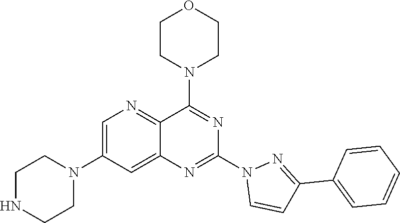 [SMILES: c1ccc(-c2ccn(-c3nc(N4CCOCC4)c4ncc(N5CCNCC5)cc4n3)n2)cc1] | ¹H NMR (400 MHz, CHLOROFORM-d) δ = 8.63 (d, J = 2.6 Hz, 1H), 8.45 (d, J = 2.9 Hz, 1H), 8.07 − 7.99 (m, 2H), 7.49 − 7.31 (m, 4H), 6.80 (d, J = 2.8 Hz, 1H), 4.58 (bs, 4H), 3.99 − 3.88 (m, 4H), 3.46 − 3.35 (m, 4H), 3.14 − 3.04 (m, 4H). LCMS (ESI) for C24H26N8O [M + H]⁺: 443.3. | 66 |

### Example 8. PIKfyve Inhibitory Activity, Metabolic Stability, Permeability, and Solubility

PIKfyve Biochemical Assay. The biochemical PIKFyve inhibition assays were run by Carna Biosciences according to proprietary methodology based on the Promega ADP-Glo™ Kinase assay. A full-length human PIKFYVE [1-2098(end) amino acids and S696N, L932S, Q995L, T998S, S1033A and Q1183K of the protein having the sequence set forth in NCBI Reference Sequence No. NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega). Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADP-Glo™ reagent was then added for 40 minutes, followed by kinase detection reagent for an additional 40 minutes. The kinase activity was evaluated by detecting relative light units on a luminescence plate reader. Samples were run in duplicate from 10 μM to 3 nM. Data was analyzed by setting the control wells (+ PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

NanoBRET™ TE Intracellular Kinase Assay, K-8 (Promega) Cell-Based Assay. Intracellular inhibition of PIKfyve was assayed using Promega's NanoBRET™ TE Intracellular Kinase Assay, K-8 according to manufacturer's instructions. A dilution series of test compounds was added for 2 hours to HEK293 cells transfected for a minimum of 20 hours with PIKFYVE-NanoLuc® Fusion Vector (Promega) containing a full-length PIKfyve according to manufacturer's specifications in a 96-well plate. Kinase activity was detected by addition of a NanoBRET™ tracer reagent, which was a proprietary PIKfyve inhibitor appended to a fluorescent probe (BRET, bioluminescence resonance energy transfer). Test compounds were tested at concentrations of 10, 3, 1, 0.3, 0.1, 0.03, 0.01, 0.003 μM. BRET signals were measured by a GloMax®Discover Multimode Microplate Reader (Promega) using 0.3 sec/well integration time, 450BP donor filter and 600LP acceptor filters. Active test compounds that bound PIKfyve and displaced the tracer reduced BRET signal. IC50 values were then calculated by fitting the data to the normalized BRET ratio.

The results of the PIKfyve inhibition assays are summarized in the Table below.

| | | hPIKfyve |
|---|---|---|
| Compound | hPIKfyve | BRET |
| No. | IC₅₀ (μM)^{a} | IC₅₀ (μM)^{a} |
| 1 | + | |
| 2 | + | |
| 3 | + | |
| 4 | + | |
| 5 | + | |
| 6 | + | |
| 7 | ++ | |
| 8 | +++ | ++ |
| 9 | + | |
| 10 | ++ | |
| 11 | ++ | |
| 12 | +++ | |
| 13 | ++ | |
| 14 | + | |
| 15 | ++ | |
| 16 | − | |
| 17 | ++ | |
| 18 | +++ | ++ |
| 19 | +++ | |
| 20 | +++ | ++ |
| 21 | +++ | +++ |
| 22 | +++ | ++ |
| 23 | ++ | |
| 24 | +++ | +++ |
| 25 | + | |
| 26 | +++ | ++ |
| 27 | +++ | |
| 28 | + | |
| 29 | + | |
| 30 | + | |
| 31 | + | |
| 32 | + | |
| 33 | + | |
| 34 | + | |
| 35 | + | |
| 36 | + | |
| 37 | + | |
| 38 | + | |
| 39 | ++++ | ++ |
| 40 | ++ | |
| 41 | − | |
| 42 | + | |
| 43 | + | |
| 44 | + | |
| 45 | + | |
| 46 | + | |
| 47 | + | |
| 48 | + | |
| 49 | | |
| 50 | | |
| 51 | + | |
| 52 | | |
| 53 | | |
| 54 | | |
| 55 | + | |
| 56 | | |
| 57 | | |
| 58 | | |
| 59 | | |
| 60 | | |
| 61 | | |
| 62 | | |
| 63 | | |
| 64 | | |
| 65 | | |
| 66 | | |
| 67 | | |
| 68 | | |
| 69 | | |
| 70 | | |
| 71 | | |
| 72 | +++ | |
| 73 | ++ | |
| 74 | ++ | |
| 75 | +++ | |
| 76 | ++ | |
| 77 | ++ | |
| 78 | ++ | |
| 79 | ++ | ++ |
| 80 | +++ | +++ |
| 81 | +++ | ++ |
| 82 | + | |
| 83 | + | |
| 84 | + | |
| 85 | ++ | |
| 86 | ++ | |
| 87 | ++ | |
| 88 | + | |

^{a}++++ stands for <10 nM, +++ stands for 10-100 nM, ++ stands for 100-1000 nM, + stands for 1-10 μM, and − stands for >10 μM.

### Example 9. Viability Assay to Assess TDP-43 Toxicity in FAB1 TDP-43 and PIKfyve TDP-43 Yeast Cells

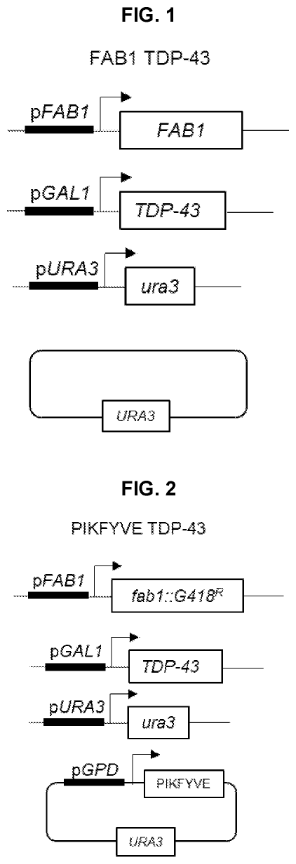

Generation of TDP-43 yeast model expressing human PIKfyve. Human PIKFYVE (“entry clone”) was cloned into pAG416GPDccdB (“destination vector”) according to standard Gateway cloning protocols (Invitrogen, Life Technologies). The resulting pAG416GPD-PIKFYVE plasmids were amplified in E. coli and plasmid identity confirmed by restriction digest and Sanger sequencing. Lithium acetate/polyethylene glycol-based transformation was used to introduce the above PIKFYVE plasmid into a BY4741 yeast strain auxotrophic for the ura3 gene and deleted for two transcription factors that regulate the xenobiotic efflux pumps, a major efflux pump, and FAB1, the yeast ortholog of PIKFYVE (MATa, snq2::KILeu2; pdr3::Klura3;pdrl::NATMX; fab1::G418^{R}, his3;leu2;ura3;met15;LYS2+) (FIG. 2). Transformed yeast were plated on solid agar plates with complete synthetic media lacking uracil (CSM-ura) and containing 2% glucose. Individual colonies harboring the control or PIKFYVE TDP-43 plasmids were recovered. A plasmid containing wild-type TDP-43 under the transcriptional control of the GAL1 promoter and containing the hygromycin-resistance gene as a selectable marker was transformed into the fab1::G418^{R} pAG416GPD-PIKFYVE yeast strain (FIG. 1). Transformed yeast were plated on CSM-ura containing 2% glucose and 200 μg/mL G418 after overnight recovery in media lacking antibiotic. Multiple independent isolates were further evaluated for cytotoxicity and TDP-43 expression levels.

Viability Assay. A control yeast strain with the wild-type yeast FAB1 gene and TDP-43 (“FAB1 TDP-43”, carries empty pAG416 plasmid), and the “PIKFYVE TDP-43” yeast strain, were assessed for toxicity using a propidium iodide viability assay. Both yeast strains were transferred from solid CSM-ura/2% glucose agar plates into 3 mL of liquid CSM-ura/2% glucose media for 6-8 hours at 30° C. with aeration. Yeast cultures were then diluted to an optical density at 600 nm wavelength (OD₆₀₀) of 0.005 in 3 mL of CSM-ura/2% raffinose and grown overnight at 30° C. with aeration to an OD₆₀₀ of 0.3-0.8. Log-phase overnight cultures were diluted to OD₆₀₀ of 0.005 in CSM-ura containing either 2% raffinose or galactose and 150 μL dispensed into each well of a flat bottom 96-well plates. Compounds formulated in 100% dimethyl sulfoxide (DMSO) were serially diluted in DMSO and 1.5 μL diluted compound transferred to the 96-well plates using a multichannel pipet. Wells containing DMSO alone were also evaluated as controls for compound effects. Tested concentrations ranged from 15 μM to 0.11 μM. Cultures were immediately mixed to ensure compound distribution and covered plates incubated at 30° C. for 24 hours in a stationary, humified incubator.

Upon the completion of incubation, cultures were assayed for viability using propidium iodide (PI) to stain for dead/dying cells. A working solution of PI was made where, for each plate, 1 μL of 10 mM PI was added to 10 mL of CSM-ura (raffinose or galactose). The final PI solution (50 μL/well) was dispensed into each well of a new round bottom 96-well plate. The overnight 96-well assay plate was then mixed with a multichannel pipet and 50 μL transferred to the PI-containing plate. This plate was then incubated for 30 minutes at 30° C. in the dark. A benchtop flow cytometer (Miltenyi MACSquant) was then used to assess red fluorescence (B2 channel), forward scatter, and side scatter (with following settings: gentle mix, high flow rate, fast measurement, 10,000 events). Intensity histograms were then gated for “PI-positive” or “PI-negative” using the raffinose and galactose cultures treated with DMSO as controls. The DMSO controls for raffinose or galactose-containing cultures were used to determine the window of increased cell death and this difference set to 100. All compounds were similarly gated and then compared to this maximal window to establish the percent reduction in PI-positive cells. IC50 values were then calculated for compounds that demonstrated a concentration-dependent enhancement of viability by fitting a logistic regression curve.

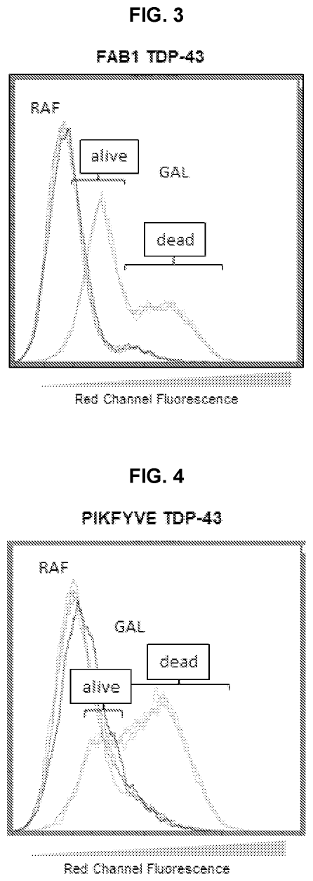

Upon induction of TDP-43 in both strains, there was a marked increase in inviable cells (rightmost population) with both FAB1 TDP-43 and PIKFYVE TDP-43, with a more pronounced effect in PIKFYVE TDP-43 (FIGS. 3 and 4).

PIKfyve Inhibition Suppresses Toxicity in PIKfyve TDP-43 Model. The biochemical PIKFyve inhibition assays were run by Carna Biosciences according to proprietary methodology based on the Promega ADP-Glo™ Kinase assay. A full-length human PIKFYVE [1-2098(end) amino acids and S696N, L932S, Q995L, T998S, S1033A and Q1183K of accession number NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega).

Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADP-Glo™ reagent was then added for 40 minutes, followed by kinase detection reagent for an additional 40 minutes. The kinase activity was evaluated by detecting relative light units on a luminescence plate reader. Samples were run in duplicate from 10 uM to 3 nM. Data was analyzed by setting the control wells (+ PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

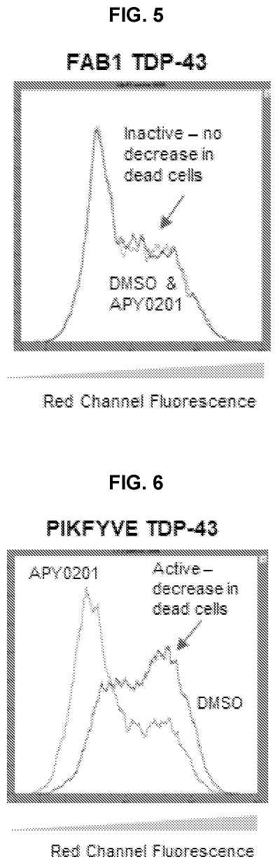

Activity of APY0201, a known PIKFYVE inhibitor, in FAB1 TDP-43 (FIG. 5) and PIKFYVE TDP-43 (FIG. 6). There was no increase in viable cells in FAB1 TDP-43 across a range of compound concentrations as evidenced by a lack in reduction of the right most population of propidium iodide-positive cells (only 0.23 μM is shown). In the PIKFYVE TDP-43 model, 0.23 μM reduced the population of propidium iodide-positive dead cells, indicating PIKFYVE inhibition ameliorated TDP-43 toxicity. Concentrations ranging from 0.5 mM to less than 100 nM afforded increased viability.

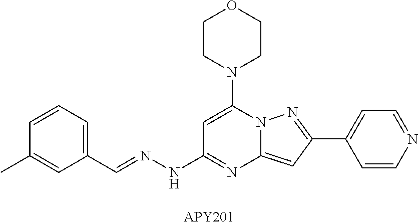 [SMILES: Cc1cccc(/C=N/Nc2cc(N3CCOCC3)n3nc(-c4ccncc4)cc3n2)c1 ; "APY201"]

A panel of compounds was tested in a biochemical PIKFYVE assay (ADP-Glo™ with full-length PIKfyve) and IC50's determined (nM) (see the Table below). The same compounds were also tested in both FAB1 and PIKFYVE TDP-43 yeast models. Their activity is reported here as “active” or “inactive.” Compounds with low nanomolar potency in the biochemical assay were active in the PIKFYVE TDP-43 yeast model. Compounds that were less potent or inactive in the biochemical assay were inactive in the PIKFYVE TDP-43 model. Compounds that were inactive in the biochemical or PIKFYVE TDP-43 assays were plotted with the highest concentrations tested in that assay.

| | | FAB1 TDP-43 | PIKfyve TDP-43 |
|---|---|---|---|
| Structure | PIKfyve IC₅₀ (nM) | (active/inactive) | (active/inactive) |
| 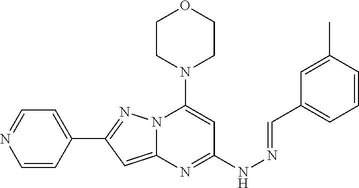 [SMILES: Cc1cccc(/C=N/Nc2cc(N3CCOCC3)n3nc(-c4ccncc4)cc3n2)c1] | 7.5 | Inactive | Active |
| 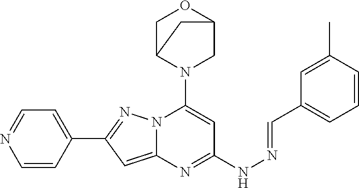 [SMILES: Cc1cccc(/C=N/Nc2cc(N3CC4CC3CO4)n3nc(-c4ccncc4)cc3n2)c1] | 12 | Inactive | Active |
| 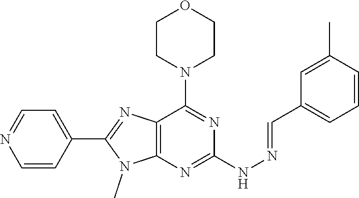 [SMILES: Cc1cccc(/C=N/Nc2nc(N3CCOCC3)c3nc(-c4ccncc4)n(C)c3n2)c1] | 4.9 | Inactive | Active |
| 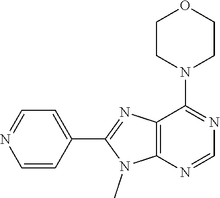 [SMILES: Cn1c(-c2ccncc2)nc2c(N3CCOCC3)ncnc21] | 640 | Inactive | Inactive |
| 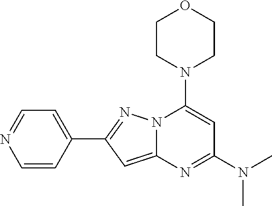 [SMILES: CN(C)c1cc(N2CCOCC2)n2nc(-c3ccncc3)cc2n1] | 2007 | Inactive | Inactive |
| 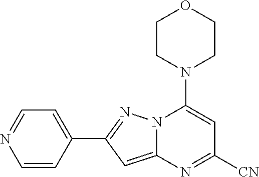 [SMILES: N#Cc1cc(N2CCOCC2)n2nc(-c3ccncc3)cc2n1] | >10000 | Inactive | Inactive |

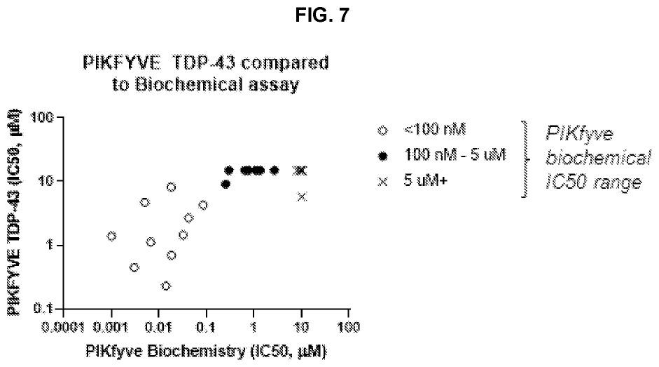

Biochemical and Efficacy Assays. A larger set of PIKfyve inhibitors were evaluated in both a PIKfyve kinase domain binding assay (nanobret) and in the PIKFYVE TDP-43 yeast strain. IC50 values (μM) were plotted. Data points are formatted based on binned potency from the nanobret assay as indicated in the legend (FIG. 7). Below is a table of compounds and their biochemical and PIKFYVE TDP-43 IC50 values plotted in FIG. 7.

| | PIKFYVE Biochemistry | PIKFYVE TDP-43 |
|---|---|---|
| Structure | (IC50, μM) | (IC50, μM) |
| 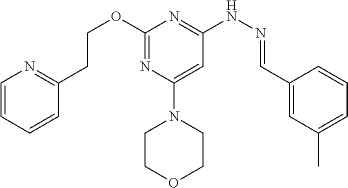 [SMILES: Cc1cccc(/C=N/Nc2cc(N3CCOCC3)nc(OCCc3ccccn3)n2)c1] | 0.003 | 0.450 |
| 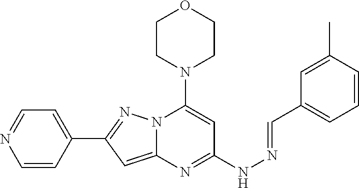 [SMILES: Cc1cccc(/C=N/Nc2cc(N3CCOCC3)n3nc(-c4ccncc4)cc3n2)c1] | 0.001 | 1.390 |
| 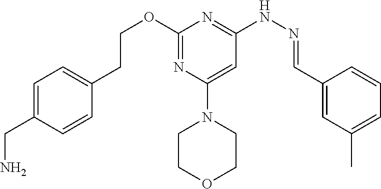 [SMILES: Cc1cccc(/C=N/Nc2cc(N3CCOCC3)nc(OCCc3ccc(CN)cc3)n2)c1] | 0.007 | 1.120 |
| 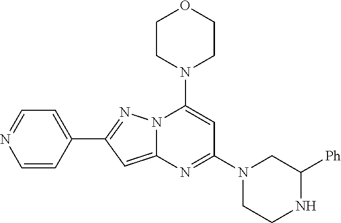 [SMILES: c1ccc(C2CN(c3cc(N4CCOCC4)n4nc(-c5ccncc5)cc4n3)CCN2)cc1] | 2.660 | >15 |
| 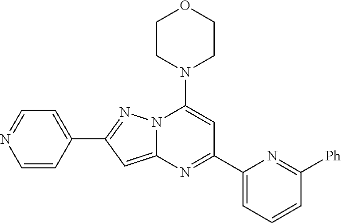 [SMILES: c1ccc(-c2cccc(-c3cc(N4CCOCC4)n4nc(-c5ccncc5)cc4n3)n2)cc1] | 0.014 | 0.230 |
| 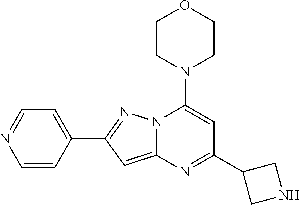 [SMILES: c1cc(-c2cc3nc(C4CNC4)cc(N4CCOCC4)n3n2)ccn1] | 8.020 | >15 |
| 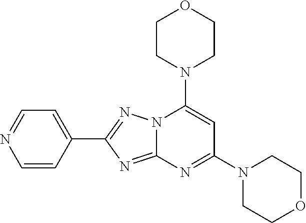 [SMILES: c1cc(-c2nc3nc(N4CCOCC4)cc(N4CCOCC4)n3n2)ccn1] | 9.200 | >15 |
| 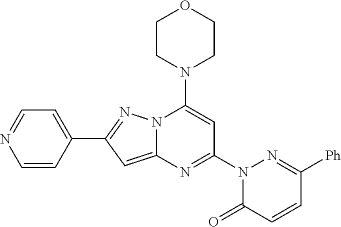 [SMILES: O=c1ccc(-c2ccccc2)nn1-c1cc(N2CCOCC2)n2nc(-c3ccncc3)cc2n1] | 0.295 | >15 |
| 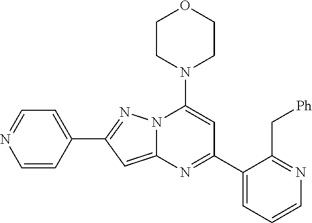 [SMILES: c1ccc(Cc2ncccc2-c2cc(N3CCOCC3)n3nc(-c4ccncc4)cc3n2)cc1] | 1.090 | >15 |
| 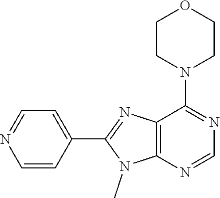 [SMILES: Cn1c(-c2ccncc2)nc2c(N3CCOCC3)ncnc21] | 0.640 | >15 |
| 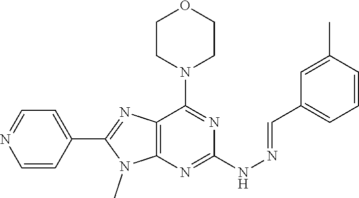 [SMILES: Cc1cccc(/C=N/Nc2nc(N3CCOCC3)c3nc(-c4ccncc4)n(C)c3n2)c1] | 0.005 | 4.720 |
| 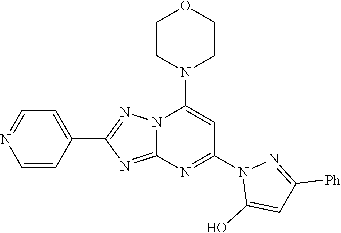 [SMILES: Oc1cc(-c2ccccc2)nn1-c1cc(N2CCOCC2)n2nc(-c3ccncc3)nc2n1] | 0.018 | 0.693 |
| 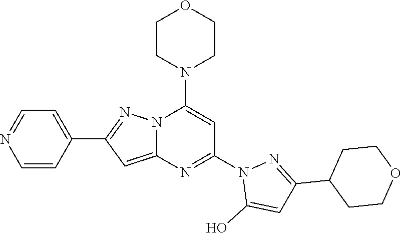 [SMILES: Oc1cc(C2CCOCC2)nn1-c1cc(N2CCOCC2)n2nc(-c3ccncc3)cc2n1] | 0.253 | 9.105 |
| 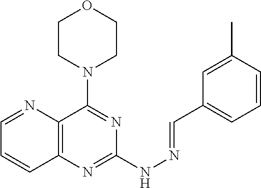 [SMILES: Cc1cccc(/C=N/Nc2nc(N3CCOCC3)c3ncccc3n2)c1] | 0.018 | 8.214 |
| 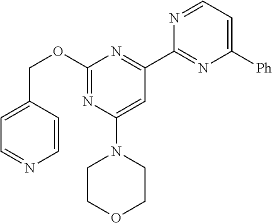 [SMILES: c1ccc(-c2ccnc(-c3cc(N4CCOCC4)nc(OCc4ccncc4)n3)n2)cc1] | 0.032 | 1.447 |
| 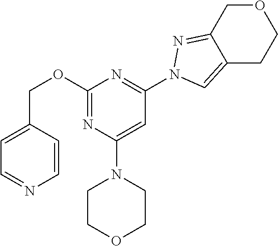 [SMILES: c1cc(COc2nc(N3CCOCC3)cc(-n3cc4c(n3)COCC4)n2)ccn1] | 1.343 | >15 |
| 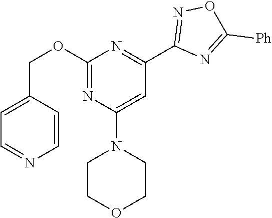 [SMILES: c1ccc(-c2nc(-c3cc(N4CCOCC4)nc(OCc4ccncc4)n3)no2)cc1] | >10 | >15 |
| 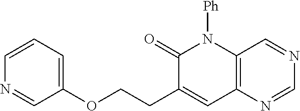 [SMILES: O=c1c(CCOc2cccnc2)cc2ncncc2n1-c1ccccc1] | >10 | >15 |
| 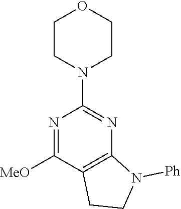 [SMILES: COc1nc(N2CCOCC2)nc2c1CCN2c1ccccc1] | 0.085 | 4.273 |
| 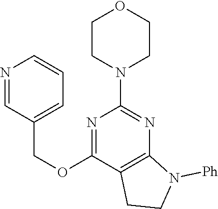 [SMILES: c1ccc(N2CCc3c(OCc4cccnc4)nc(N4CCOCC4)nc32)cc1] | 0.042 | 2.685 |
| 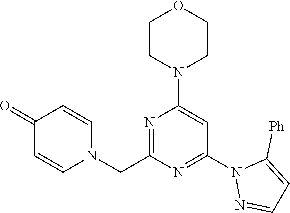 [SMILES: O=c1ccn(Cc2nc(N3CCOCC3)cc(-n3nccc3-c3ccccc3)n2)cc1] | >10 | >15 |
| 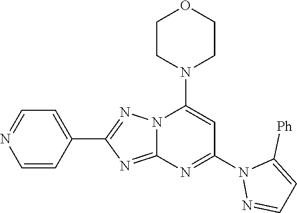 [SMILES: c1ccc(-c2ccnn2-c2cc(N3CCOCC3)n3nc(-c4ccncc4)nc3n2)cc1] | 0.767 | >15 |
| 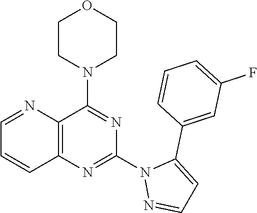 [SMILES: Fc1cccc(-c2ccnn2-c2nc(N3CCOCC3)c3ncccc3n2)c1] | >10 | 5.754 |

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1-215. (canceled)

216. A compound chosen from compounds of Formula I or pharmaceutically acceptable salts thereof: 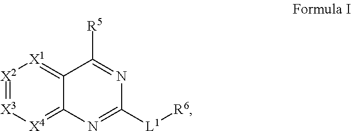
wherein:
X¹ is chosen from N or CR¹;
X² is chosen from N or CR²;
X³ is chosen from N or CR³;
X⁴ is chosen from N or CR⁴;
R⁵ is chosen from one of the following: 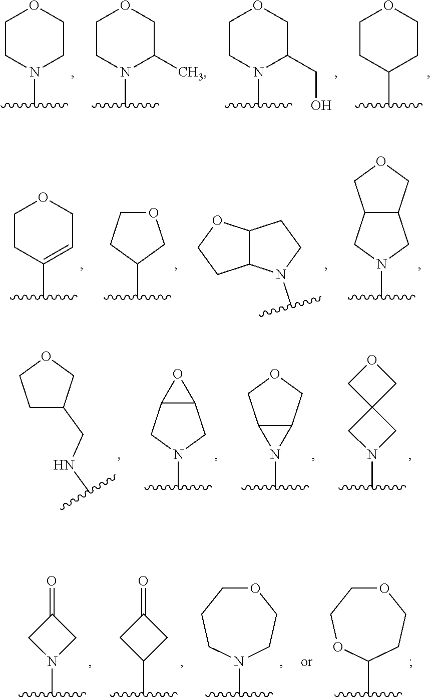
L¹ is chosen from an optionally substituted C₁₋₉ heteroarylene group having at least one 5-membered ring, an optionally substituted non-aromatic C₁₋₉ heterocyclylene group, or one of the following: 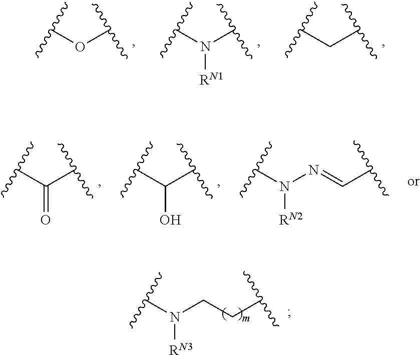
R⁶ is chosen from an optionally substituted C₁₋₆ alkyl group, an optionally substituted C₆₋₁₀ aryl group, an optionally substituted C₃₋₁₀ carbocyclyl group, an optionally substituted C₁₋₉ heteroaryl group, an optionally substituted C₁₋₉ heterocyclyl group, or an optionally substituted C₁₋₆ alkylene-C₁₋₉ heterocyclyl group;
alternatively, L¹ and R⁶ together may form an optionally substituted C₂₋₉ oxyheteroaryl group, an optionally substituted pyrimidin-r-yl group, or an optionally substituted pyrid-2-yl group;
R¹ is chosen from hydrogen, a halogen atom, or an optionally substituted C₁₋₆ alkyl group;
R² is hydrogen;
R³ is chosen from hydrogen, a halogen atom, an optionally substituted C₁₋₆ alkyl group, or 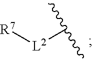
R⁴ is chosen from hydrogen, a halogen atom, or an optionally substituted C₁₋₆ alkyl group;
L² is absent or is chosen from one of the following: 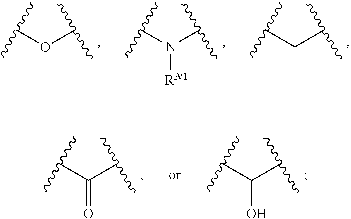
R⁷ is chosen from an optionally substituted C₆₋₁₀ aryl group, an optionally substituted C₃₋₁₀ carbocyclyl group, an optionally substituted C₁₋₉ heteroaryl group, or an optionally substituted C₁₋₉ heterocyclyl group;
R^{N1}, R^{N2}, and R^{N3}, which may be the same or different, are independently chosen from hydrogen or an optionally substituted C₁₋₆ alkyl group; and
m is 0, 1, 2, or 3;
with the proviso that only one of X¹, X², X³, and X⁴ is N.

217. A composition comprising a compound according to claim 216 and a pharmaceutically acceptable excipient.

218. A method of treating a neurological disorder in a patient in need thereof, the method comprising administering to the patient an effective amount of a compound according to claim 216 to the patient.

219. The method according to claim 218, wherein the neurological disorder is FTLD-TDP, chronic traumatic encephalopathy, ALS, Alzheimer's disease, LATE, or frontotemporal lobar degeneration.

220. A method of inhibiting toxicity in a cell related to a protein, the method comprising contacting the cell with a compound according to claim 216.

221. The method according to claim 220, wherein the toxicity is TDP-43-related toxicity or C9orf72-related toxicity.

222. A method of inhibiting PIKfyve in a cell expressing PIKfyve protein, the method comprising contacting the cell with a compound according to claim 216.

223. A compound chosen from compounds of Formula II, Formula III, Formula IV, Formula V, or pharmaceutically acceptable salts thereof: 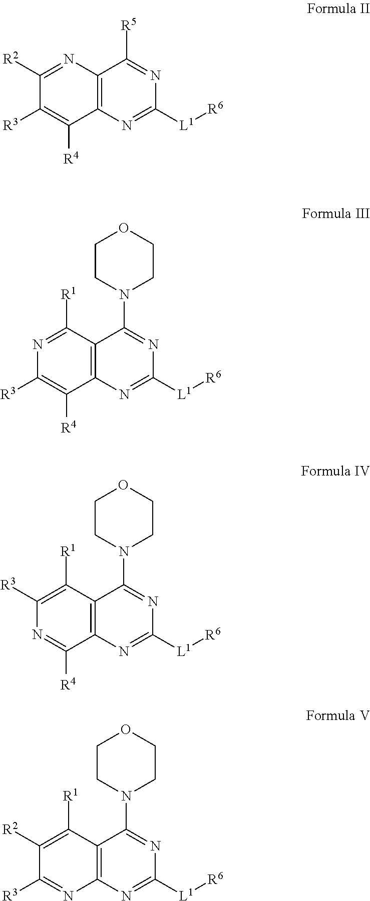
wherein:
R¹ is chosen from hydrogen, a halogen atom, or an optionally substituted C₁₋₆ alkyl group;
R² is chosen from hydrogen, a halogen atom, or an optionally substituted C₁₋₆ alkyl group;
R³ is chosen from hydrogen, a halogen atom, an optionally substituted C₁₋₆ alkyl group, or 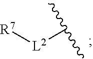
R⁴ is chosen from hydrogen, a halogen atom, or an optionally substituted C₁₋₆ alkyl group;
R⁵ is chosen from one of the following: 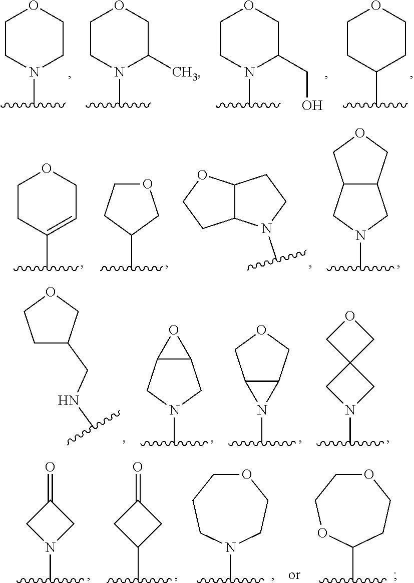
L¹ is chosen from an optionally substituted C₁₋₉ heteroarylene group having at least one 5-membered ring, an optionally substituted non-aromatic C₁₋₉ heterocyclylene group, or one of the following: 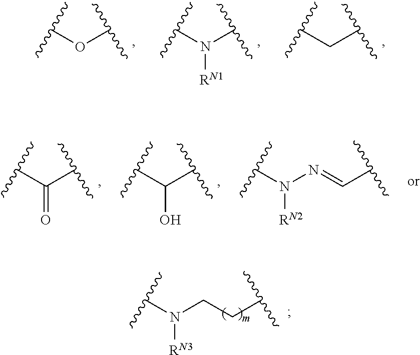
R⁶ is chosen from an optionally substituted C₁₋₆ alkyl group, an optionally substituted C₆₋₁₀ aryl group, an optionally substituted C₃₋₁₀ carbocyclyl group, an optionally substituted C₁₋₉ heteroaryl group, an optionally substituted C₁₋₉ heterocyclyl group, or an optionally substituted C₁₋₆ alkylene-C₁₋₅ heterocyclyl group;
alternatively, L¹ and R⁶ together may form an optionally substituted C₂₋₉ oxyheteroaryl group, an optionally substituted pyrimidin-r-yl group, or an optionally substituted pyrid-2-yl group;
L² is absent or is chosen from one of the following: 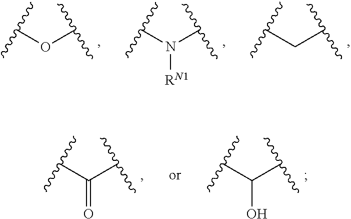
R⁷ is chosen from an optionally substituted C₆₋₁₀ aryl group, an optionally substituted C₃₋₁₀ carbocyclyl group, an optionally substituted C₁₋₉ heteroaryl group, or an optionally substituted C₁₋₉ heterocyclyl group;
R^{N1}, R^{N2}, and R^{N3}, which may be the same or different, are independently chosen from hydrogen or an optionally substituted C₁₋₆ alkyl group; and
m is 0, 1, 2, or 3.

224. A composition comprising a compound according to claim 223 and a pharmaceutically acceptable excipient.

225. A method of treating a neurological disorder in a patient in need thereof, the method comprising administering to the patient an effective amount of a compound according to claim 223 to the patient.

226. The method according to claim 225, wherein the neurological disorder is FTLD-TDP, chronic traumatic encephalopathy, ALS, Alzheimer's disease, LATE, or frontotemporal lobar degeneration.

227. A method of inhibiting toxicity in a cell related to a protein, the method comprising contacting the cell with a compound according to claim 223.

228. The method according to claim 227, wherein the toxicity is TDP-43-related toxicity or C9orf72-related toxicity.

229. A method of inhibiting PIKfyve in a cell expressing PIKfyve protein, the method comprising contacting the cell with a compound according to claim 223.

230. A compound chosen from compounds 1-88 or pharmaceutically acceptable salts thereof:

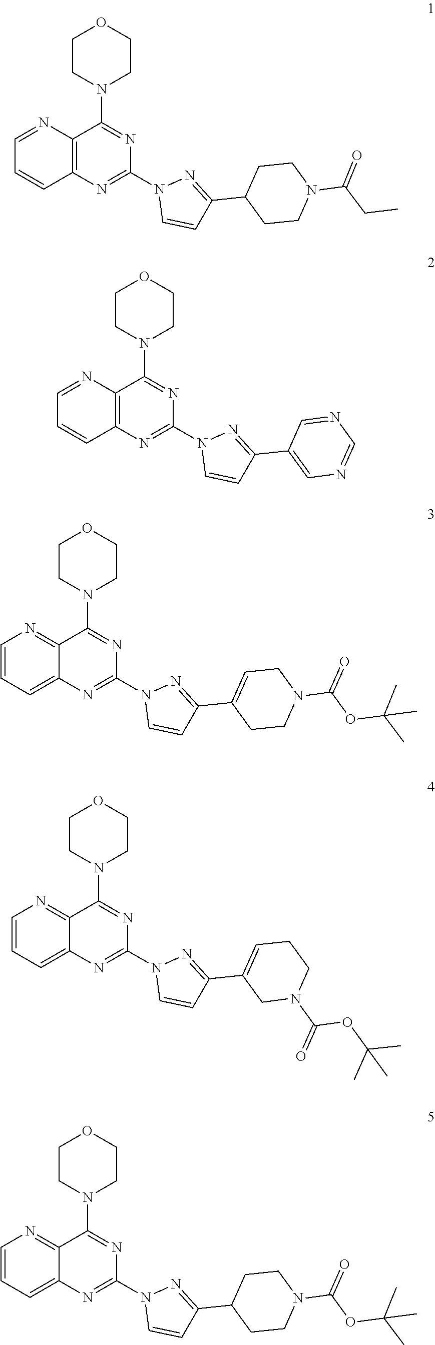 [SMILES: CCC(=O)N1CCC(c2ccn(-c3nc(N4CCOCC4)c4ncccc4n3)n2)CC1 ; c1cnc2c(N3CCOCC3)nc(-n3ccc(-c4cncnc4)n3)nc2c1 ; CC(C)(C)OC(=O)N1CC=C(c2ccn(-c3nc(N4CCOCC4)c4ncccc4n3)n2)CC1 ; CC(C)(C)OC(=O)N1CCC=C(c2ccn(-c3nc(N4CCOCC4)c4ncccc4n3)n2)C1 ; CC(C)(C)OC(=O)N1CCC(c2ccn(-c3nc(N4CCOCC4)c4ncccc4n3)n2)CC1]

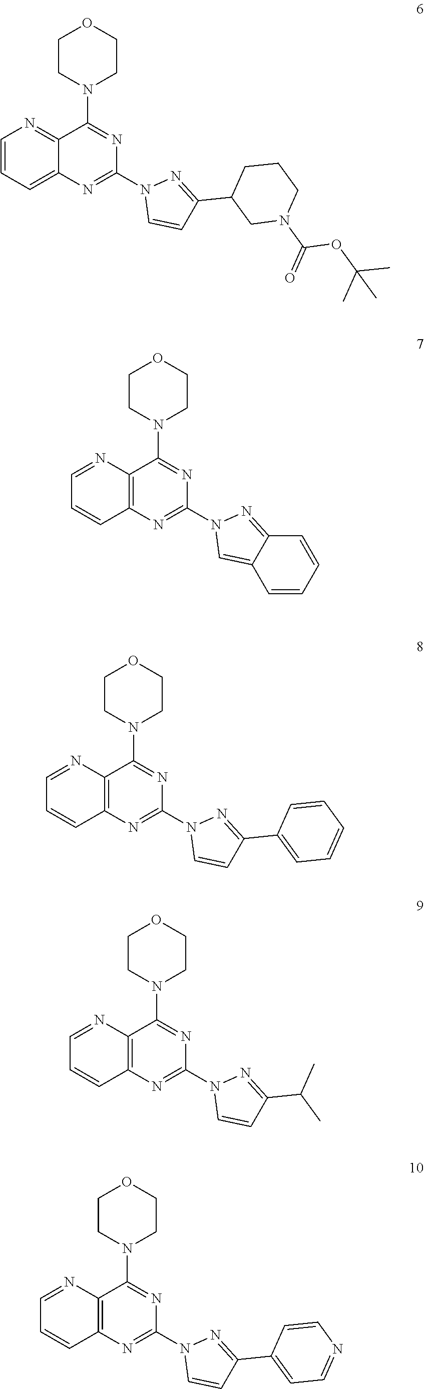 [SMILES: CC(C)(C)OC(=O)N1CCCC(c2ccn(-c3nc(N4CCOCC4)c4ncccc4n3)n2)C1 ; c1ccc2nn(-c3nc(N4CCOCC4)c4ncccc4n3)cc2c1 ; c1ccc(-c2ccn(-c3nc(N4CCOCC4)c4ncccc4n3)n2)cc1 ; CC(C)c1ccn(-c2nc(N3CCOCC3)c3ncccc3n2)n1 ; c1cnc2c(N3CCOCC3)nc(-n3ccc(-c4ccncc4)n3)nc2c1]

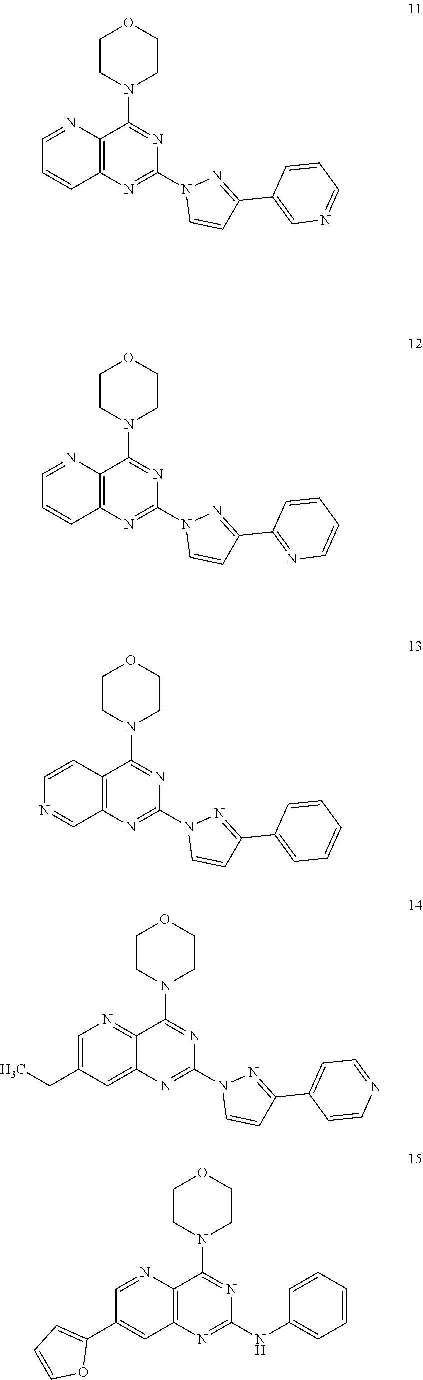 [SMILES: c1cncc(-c2ccn(-c3nc(N4CCOCC4)c4ncccc4n3)n2)c1 ; c1ccc(-c2ccn(-c3nc(N4CCOCC4)c4ncccc4n3)n2)nc1 ; c1ccc(-c2ccn(-c3nc(N4CCOCC4)c4ccncc4n3)n2)cc1 ; CCc1cnc2c(N3CCOCC3)nc(-n3ccc(-c4ccncc4)n3)nc2c1 ; c1ccc(Nc2nc(N3CCOCC3)c3ncc(-c4ccco4)cc3n2)cc1]

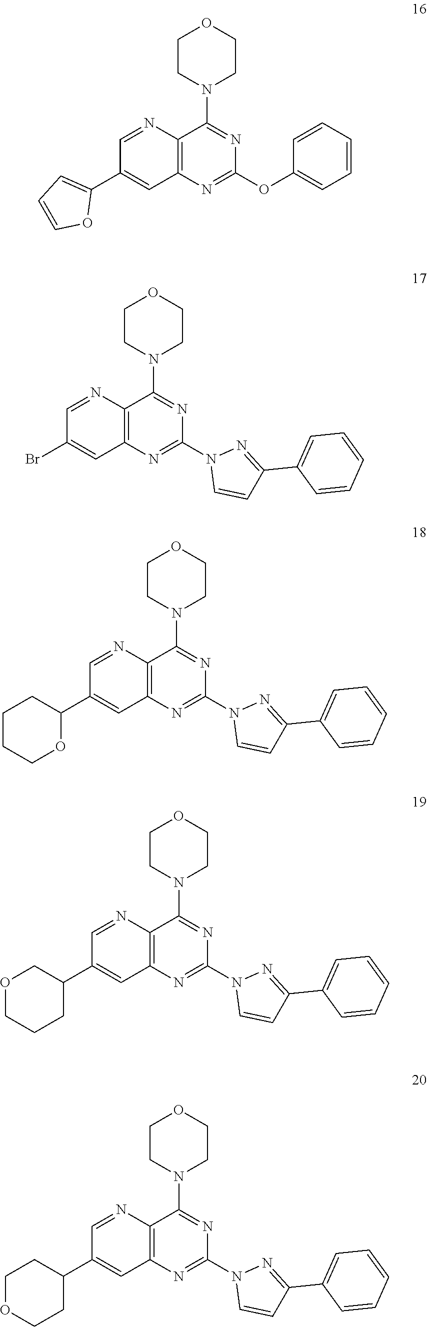 [SMILES: c1ccc(Oc2nc(N3CCOCC3)c3ncc(-c4ccco4)cc3n2)cc1 ; Brc1cnc2c(N3CCOCC3)nc(-n3ccc(-c4ccccc4)n3)nc2c1 ; c1ccc(-c2ccn(-c3nc(N4CCOCC4)c4ncc(C5CCCCO5)cc4n3)n2)cc1 ; c1ccc(-c2ccn(-c3nc(N4CCOCC4)c4ncc(C5CCCOC5)cc4n3)n2)cc1 ; c1ccc(-c2ccn(-c3nc(N4CCOCC4)c4ncc(C5CCOCC5)cc4n3)n2)cc1]

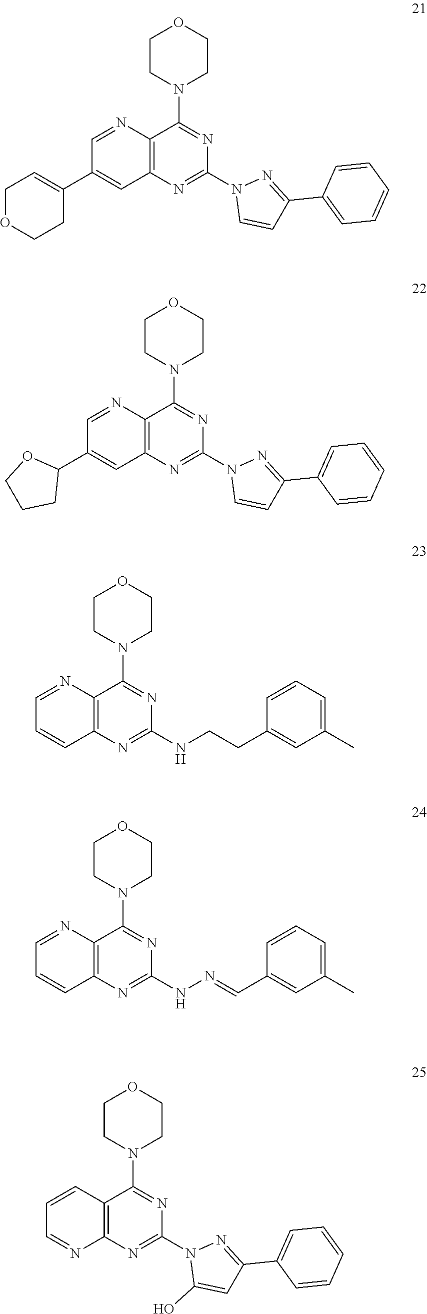 [SMILES: C1=C(c2cnc3c(N4CCOCC4)nc(-n4ccc(-c5ccccc5)n4)nc3c2)CCOC1 ; c1ccc(-c2ccn(-c3nc(N4CCOCC4)c4ncc(C5CCCO5)cc4n3)n2)cc1 ; Cc1cccc(CCNc2nc(N3CCOCC3)c3ncccc3n2)c1 ; Cc1cccc(/C=N/Nc2nc(N3CCOCC3)c3ncccc3n2)c1 ; Oc1cc(-c2ccccc2)nn1-c1nc(N2CCOCC2)c2cccnc2n1]

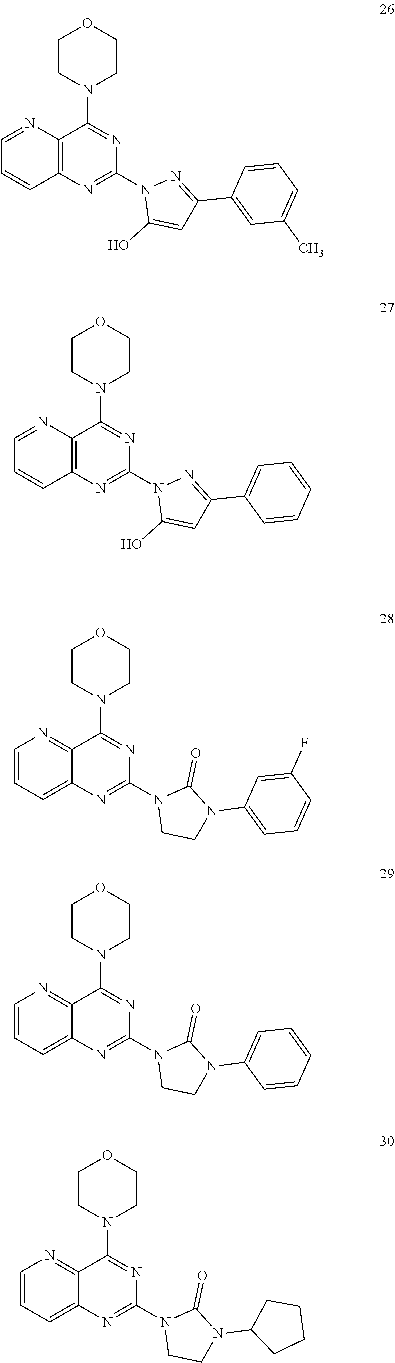 [SMILES: Cc1cccc(-c2cc(O)n(-c3nc(N4CCOCC4)c4ncccc4n3)n2)c1 ; Oc1cc(-c2ccccc2)nn1-c1nc(N2CCOCC2)c2ncccc2n1 ; O=C1N(c2cccc(F)c2)CCN1c1nc(N2CCOCC2)c2ncccc2n1 ; O=C1N(c2ccccc2)CCN1c1nc(N2CCOCC2)c2ncccc2n1 ; O=C1N(c2nc(N3CCOCC3)c3ncccc3n2)CCN1C1CCCC1]

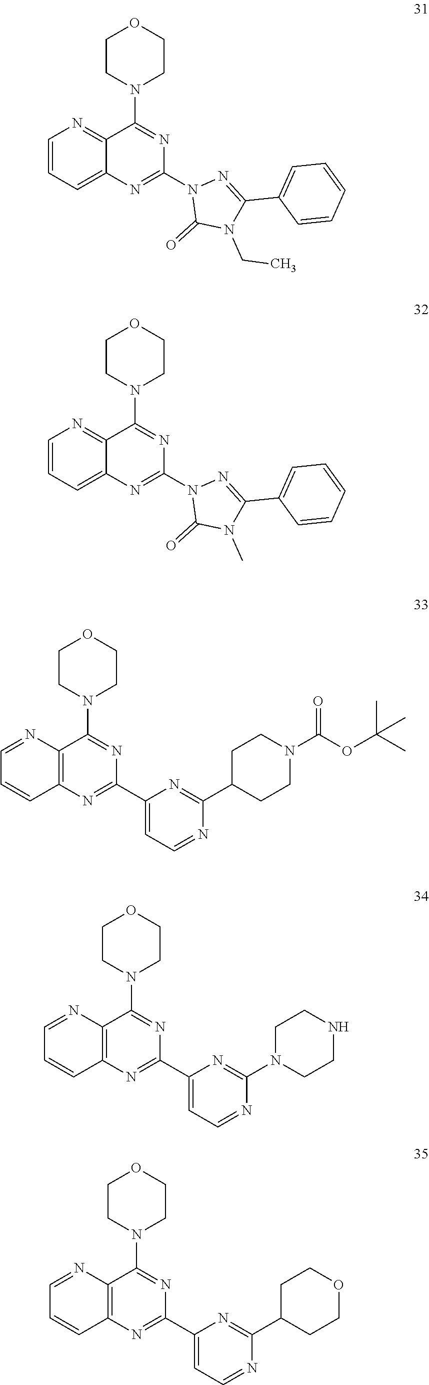 [SMILES: CCn1c(-c2ccccc2)nn(-c2nc(N3CCOCC3)c3ncccc3n2)c1=O ; Cn1c(-c2ccccc2)nn(-c2nc(N3CCOCC3)c3ncccc3n2)c1=O ; CC(C)(C)OC(=O)N1CCC(c2nccc(-c3nc(N4CCOCC4)c4ncccc4n3)n2)CC1 ; c1cnc2c(N3CCOCC3)nc(-c3ccnc(N4CCNCC4)n3)nc2c1 ; c1cnc2c(N3CCOCC3)nc(-c3ccnc(C4CCOCC4)n3)nc2c1]

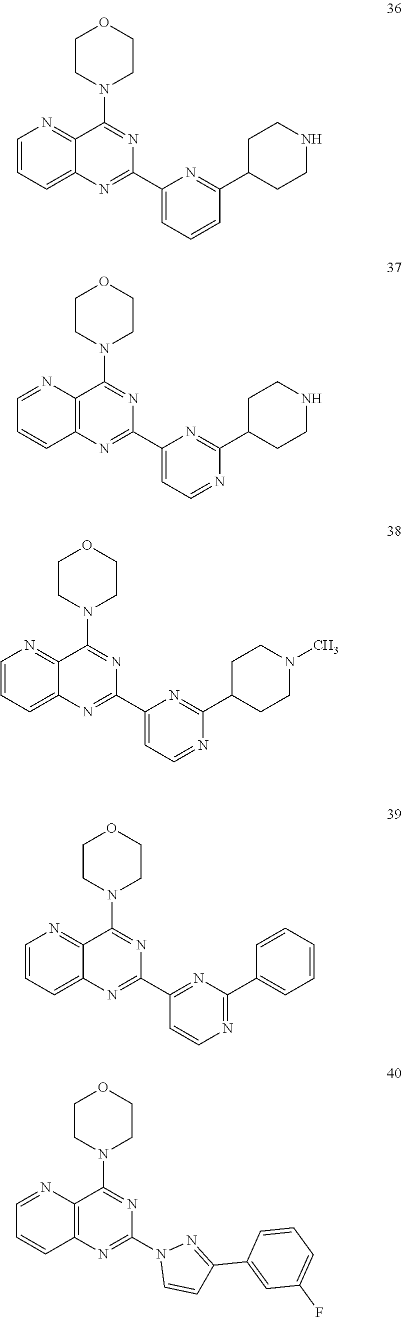 [SMILES: c1cc(-c2nc(N3CCOCC3)c3ncccc3n2)nc(C2CCNCC2)c1 ; c1cnc2c(N3CCOCC3)nc(-c3ccnc(C4CCNCC4)n3)nc2c1 ; CN1CCC(c2nccc(-c3nc(N4CCOCC4)c4ncccc4n3)n2)CC1 ; c1ccc(-c2nccc(-c3nc(N4CCOCC4)c4ncccc4n3)n2)cc1 ; Fc1cccc(-c2ccn(-c3nc(N4CCOCC4)c4ncccc4n3)n2)c1]

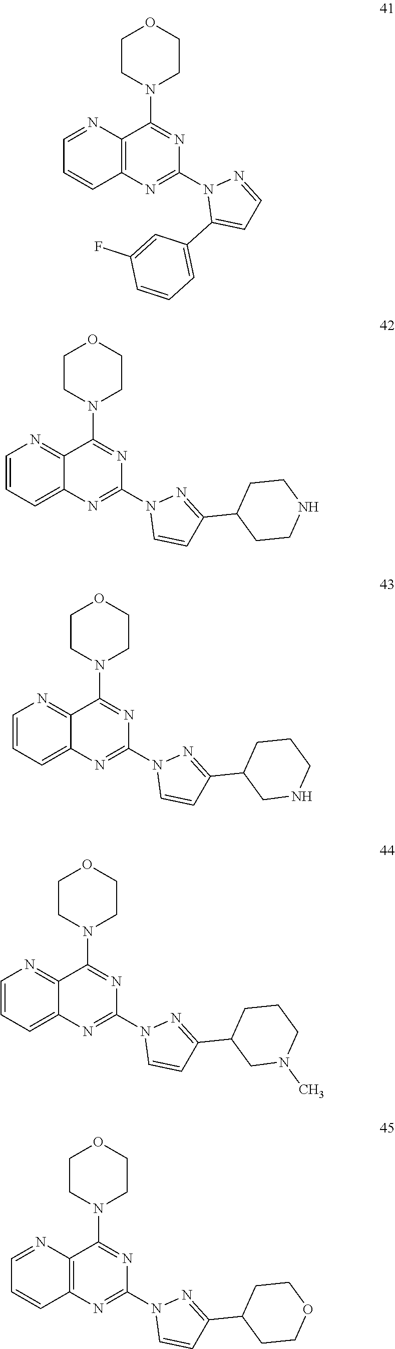 [SMILES: Fc1cccc(-c2ccnn2-c2nc(N3CCOCC3)c3ncccc3n2)c1 ; c1cnc2c(N3CCOCC3)nc(-n3ccc(C4CCNCC4)n3)nc2c1 ; c1cnc2c(N3CCOCC3)nc(-n3ccc(C4CCCNC4)n3)nc2c1 ; CN1CCCC(c2ccn(-c3nc(N4CCOCC4)c4ncccc4n3)n2)C1 ; c1cnc2c(N3CCOCC3)nc(-n3ccc(C4CCOCC4)n3)nc2c1]

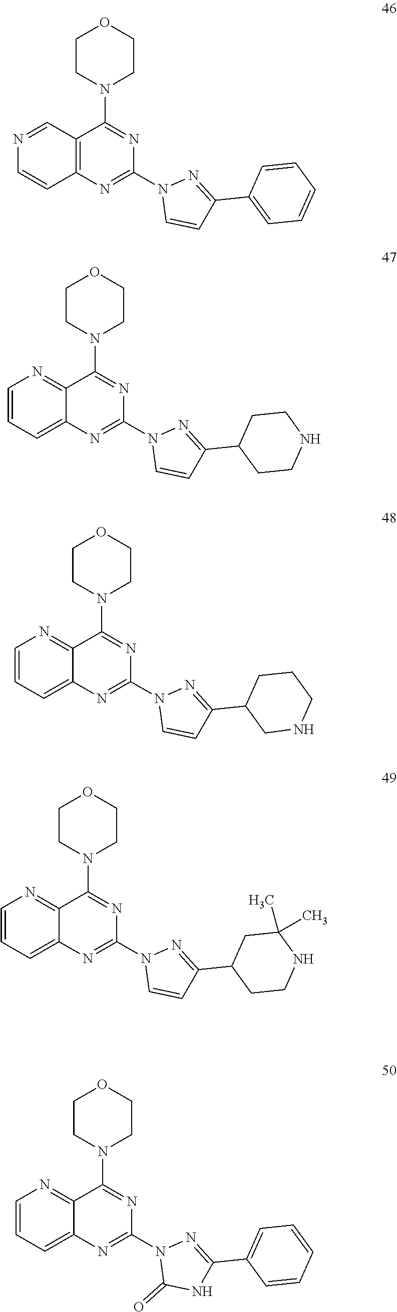 [SMILES: c1ccc(-c2ccn(-c3nc(N4CCOCC4)c4cnccc4n3)n2)cc1 ; c1cnc2c(N3CCOCC3)nc(-n3ccc(C4CCNCC4)n3)nc2c1 ; c1cnc2c(N3CCOCC3)nc(-n3ccc(C4CCCNC4)n3)nc2c1 ; CC1(C)CC(c2ccn(-c3nc(N4CCOCC4)c4ncccc4n3)n2)CCN1 ; O=c1[nH]c(-c2ccccc2)nn1-c1nc(N2CCOCC2)c2ncccc2n1]

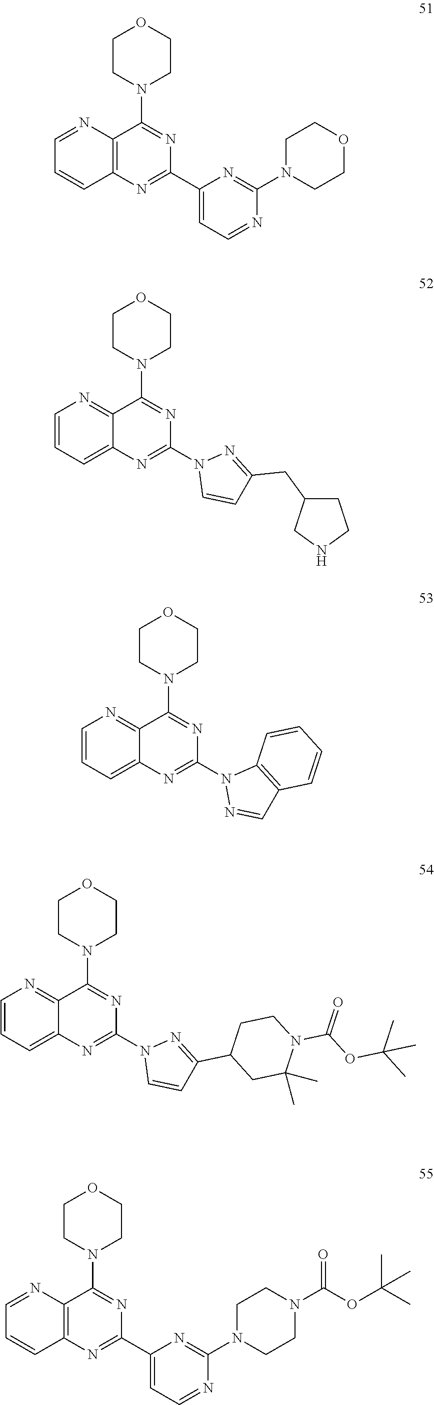 [SMILES: c1cnc2c(N3CCOCC3)nc(-c3ccnc(N4CCOCC4)n3)nc2c1 ; c1cnc2c(N3CCOCC3)nc(-n3ccc(CC4CCNC4)n3)nc2c1 ; c1ccc2c(c1)cnn2-c1nc(N2CCOCC2)c2ncccc2n1 ; CC(C)(C)OC(=O)N1CCC(c2ccn(-c3nc(N4CCOCC4)c4ncccc4n3)n2)CC1(C)C ; CC(C)(C)OC(=O)N1CCN(c2nccc(-c3nc(N4CCOCC4)c4ncccc4n3)n2)CC1]

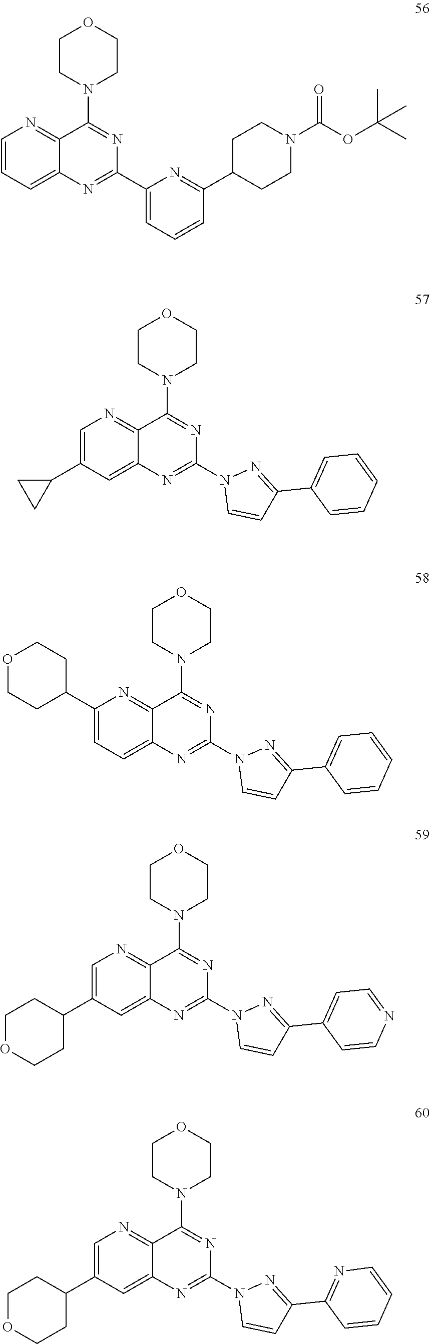 [SMILES: CC(C)(C)OC(=O)N1CCC(c2cccc(-c3nc(N4CCOCC4)c4ncccc4n3)n2)CC1 ; c1ccc(-c2ccn(-c3nc(N4CCOCC4)c4ncc(C5CC5)cc4n3)n2)cc1 ; c1ccc(-c2ccn(-c3nc(N4CCOCC4)c4nc(C5CCOCC5)ccc4n3)n2)cc1 ; c1cc(-c2ccn(-c3nc(N4CCOCC4)c4ncc(C5CCOCC5)cc4n3)n2)ccn1 ; c1ccc(-c2ccn(-c3nc(N4CCOCC4)c4ncc(C5CCOCC5)cc4n3)n2)nc1]

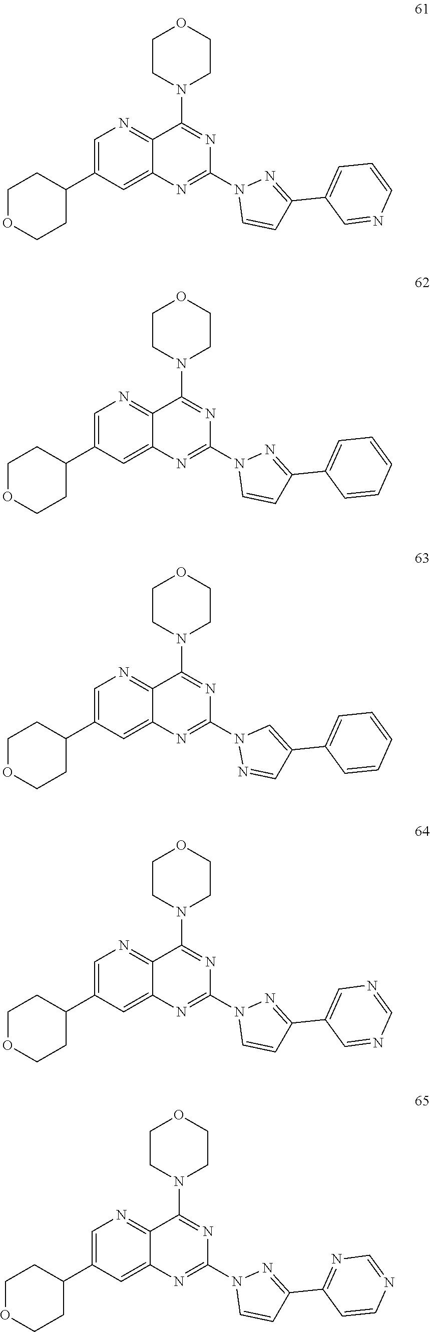 [SMILES: c1cncc(-c2ccn(-c3nc(N4CCOCC4)c4ncc(C5CCOCC5)cc4n3)n2)c1 ; c1ccc(-c2ccn(-c3nc(N4CCOCC4)c4ncc(C5CCOCC5)cc4n3)n2)cc1 ; c1ccc(-c2cnn(-c3nc(N4CCOCC4)c4ncc(C5CCOCC5)cc4n3)c2)cc1 ; c1ncc(-c2ccn(-c3nc(N4CCOCC4)c4ncc(C5CCOCC5)cc4n3)n2)cn1 ; c1cc(-c2ccn(-c3nc(N4CCOCC4)c4ncc(C5CCOCC5)cc4n3)n2)ncn1]

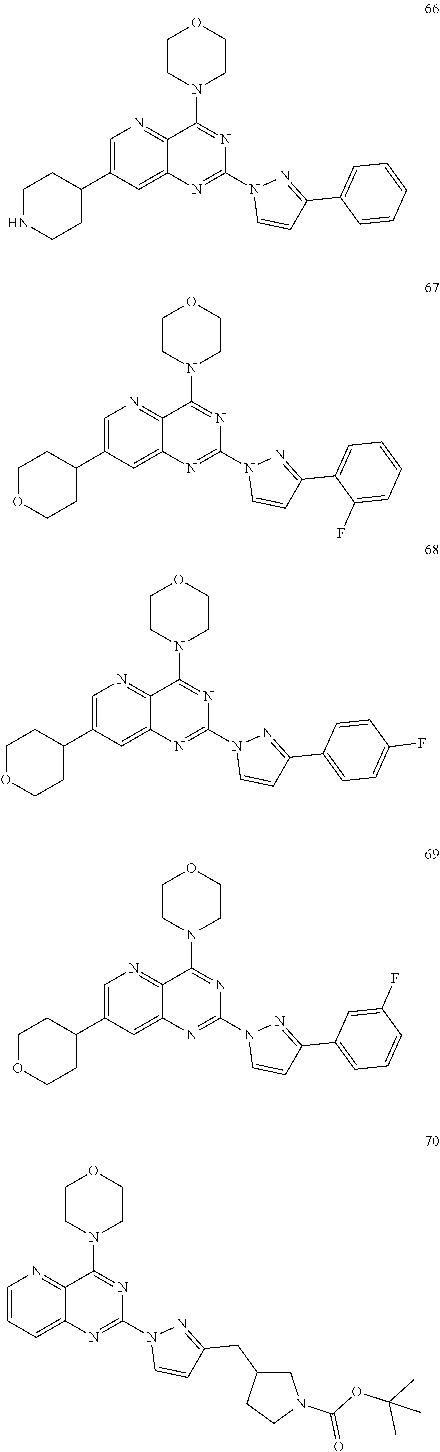 [SMILES: c1ccc(-c2ccn(-c3nc(N4CCOCC4)c4ncc(C5CCNCC5)cc4n3)n2)cc1 ; Fc1ccccc1-c1ccn(-c2nc(N3CCOCC3)c3ncc(C4CCOCC4)cc3n2)n1 ; Fc1ccc(-c2ccn(-c3nc(N4CCOCC4)c4ncc(C5CCOCC5)cc4n3)n2)cc1 ; Fc1cccc(-c2ccn(-c3nc(N4CCOCC4)c4ncc(C5CCOCC5)cc4n3)n2)c1 ; CC(C)(C)OC(=O)N1CCC(Cc2ccn(-c3nc(N4CCOCC4)c4ncccc4n3)n2)C1]

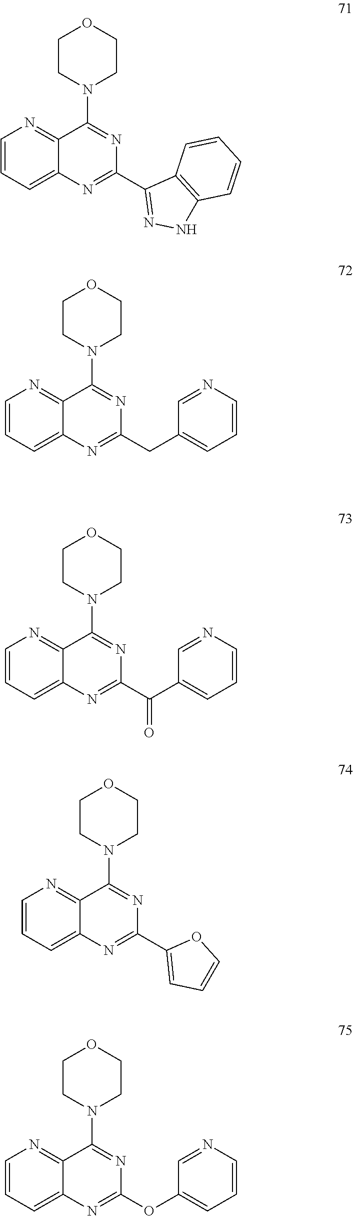 [SMILES: c1cnc2c(N3CCOCC3)nc(-c3n[nH]c4ccccc34)nc2c1 ; c1cncc(Cc2nc(N3CCOCC3)c3ncccc3n2)c1 ; O=C(c1cccnc1)c1nc(N2CCOCC2)c2ncccc2n1 ; c1coc(-c2nc(N3CCOCC3)c3ncccc3n2)c1 ; c1cncc(Oc2nc(N3CCOCC3)c3ncccc3n2)c1]

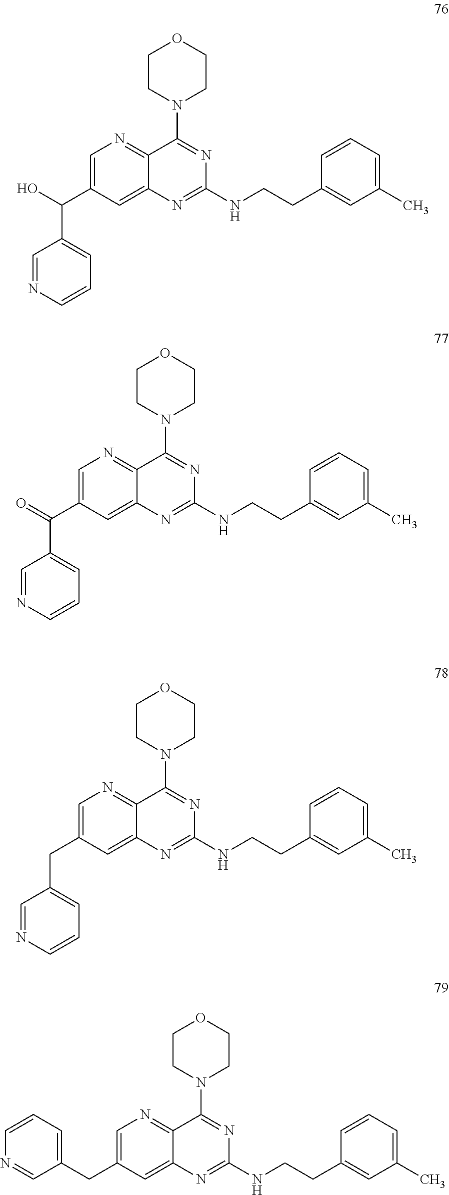 [SMILES: Cc1cccc(CCNc2nc(N3CCOCC3)c3ncc(C(O)c4cccnc4)cc3n2)c1 ; Cc1cccc(CCNc2nc(N3CCOCC3)c3ncc(C(=O)c4cccnc4)cc3n2)c1 ; Cc1cccc(CCNc2nc(N3CCOCC3)c3ncc(Cc4cccnc4)cc3n2)c1 ; Cc1cccc(CCNc2nc(N3CCOCC3)c3ncc(Cc4cccnc4)cc3n2)c1]

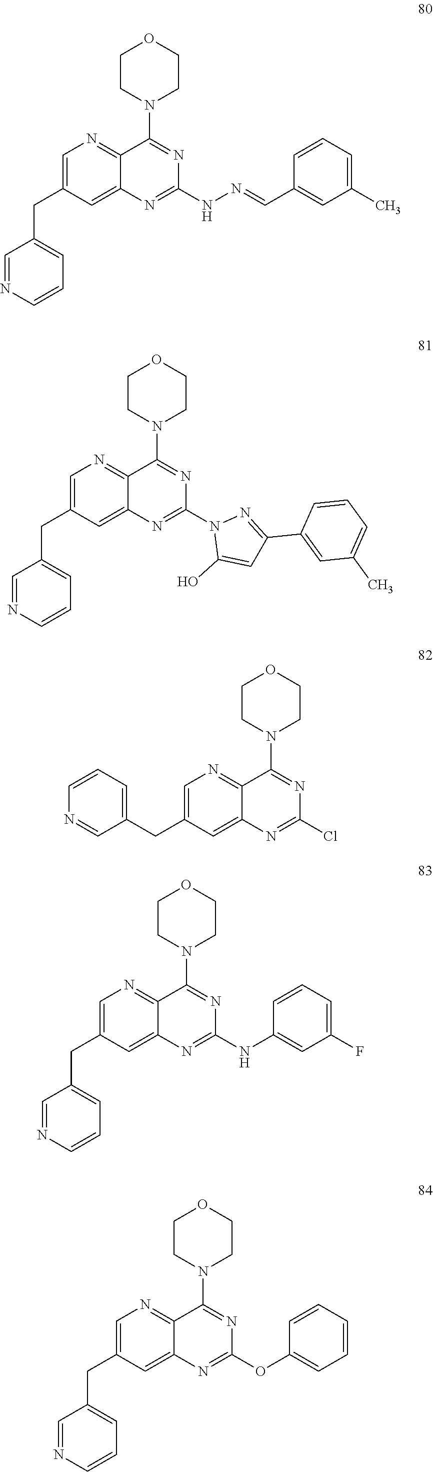 [SMILES: Cc1cccc(/C=N/Nc2nc(N3CCOCC3)c3ncc(Cc4cccnc4)cc3n2)c1 ; Cc1cccc(-c2cc(O)n(-c3nc(N4CCOCC4)c4ncc(Cc5cccnc5)cc4n3)n2)c1 ; Clc1nc(N2CCOCC2)c2ncc(Cc3cccnc3)cc2n1 ; Fc1cccc(Nc2nc(N3CCOCC3)c3ncc(Cc4cccnc4)cc3n2)c1 ; c1ccc(Oc2nc(N3CCOCC3)c3ncc(Cc4cccnc4)cc3n2)cc1]

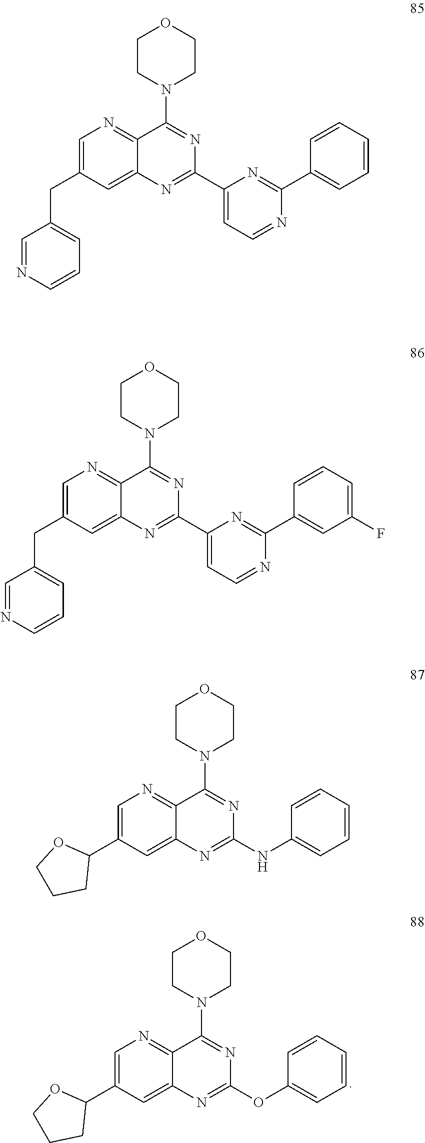 [SMILES: c1ccc(-c2nccc(-c3nc(N4CCOCC4)c4ncc(Cc5cccnc5)cc4n3)n2)cc1 ; Fc1cccc(-c2nccc(-c3nc(N4CCOCC4)c4ncc(Cc5cccnc5)cc4n3)n2)c1 ; c1ccc(Nc2nc(N3CCOCC3)c3ncc(C4CCCO4)cc3n2)cc1 ; c1ccc(Oc2nc(N3CCOCC3)c3ncc(C4CCCO4)cc3n2)cc1]

231. A composition comprising a compound according to claim 230 and a pharmaceutically acceptable excipient.

232. A method of treating a neurological disorder in a patient in need thereof, the method comprising administering to the patient an effective amount of a compound according to claim 230 to the patient.

233. The method according to claim 232, wherein the neurological disorder is FTLD-TDP, chronic traumatic encephalopathy, ALS, Alzheimer's disease, LATE, or frontotemporal lobar degeneration.

234. A method of inhibiting toxicity in a cell related to a protein, the method comprising contacting the cell with a compound according to claim 230.

235. The method according to claim 234, wherein the toxicity is TDP-43-related toxicity or C9orf72-related toxicity.

236. A method of inhibiting PIKfyve in a cell expressing PIKfyve protein, the method comprising contacting the cell with a compound according to claim 230.
